SEQUENCE LISTING (42 sequences)

SEQ ID NO: 1 - protein, 10 aa, Artificial Sequence
  Synthetic Construct
GFTFSDSWIH

SEQ ID NO: 2 - protein, 18 aa, Artificial Sequence
  Synthetic Construct
AWISPYGGSTYYADSVKG

SEQ ID NO: 3 - protein, 9 aa, Artificial Sequence
  Synthetic Construct
RHWPGGFDY

SEQ ID NO: 4 - protein, 11 aa, Artificial Sequence
  Synthetic Construct
RASQDVSTAVA

SEQ ID NO: 5 - protein, 7 aa, Artificial Sequence
  Synthetic Construct
SASFLYS

SEQ ID NO: 6 - protein, 9 aa, Artificial Sequence
  Synthetic Construct
QQYLYHPAT

SEQ ID NO: 7 - protein, 118 aa, Artificial Sequence [sequence omitted]

SEQ ID NO: 8 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 9 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 439 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 450 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 215 aa, Artificial Sequence
  Synthetic Construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - RNA, 129 nt, Artificial Sequence
  Synthetic Construct
ggcgaacuaguauucuucugguccccacagacucagagagaacccgccaccaugagagugauggcccccagaacccugauccugcugcugucuggcgcccuggcccugacagagacaugggccggaagc

SEQ ID NO: 20 - RNA, 488 nt, Artificial Sequence
  Synthetic Construct
aucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcu

SEQ ID NO: 21 - RNA, 51 nt, Artificial Sequence
  Synthetic Construct
ggcgaacuaguauucuucugguccccacagacucagagagaacccgccacc

SEQ ID NO: 22 - DNA, 51 bp, Artificial Sequence
  Synthetic Construct
ggcgaactagtattcttctggtccccacagactcagagagaacccgccacc

SEQ ID NO: 23 - RNA, 39 nt, Artificial Sequence
  Synthetic Construct
uucuucugguccccacagacucagagagaacccgccacc

SEQ ID NO: 24 - DNA, 39 bp, Artificial Sequence
  Synthetic Construct
ttcttctggtccccacagactcagagagaacccgccacc

SEQ ID NO: 25 - RNA, 78 nt, Artificial Sequence
  Synthetic Construct
augagagugauggcccccagaacccugauccugcugcugucuggcgcccuggcccugacagagacaugggccggaagc

SEQ ID NO: 26 - DNA, 78 bp, Artificial Sequence
  Synthetic Construct
atgagagtgatggcccccagaaccctgatcctgctgctgtctggcgccctggccctgacagagacatgggccggaagc

SEQ ID NO: 27 - protein, 26 aa, Artificial Sequence
  Synthetic Construct
MRVMAPRTLILLLSGALALTETWAGS

SEQ ID NO: 28 - RNA, 165 nt, Artificial Sequence
  Synthetic Construct
aucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagcc

SEQ ID NO: 29 - DNA, 165 bp, Artificial Sequence
  Synthetic Construct
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 30 - protein, 55 aa, Artificial Sequence
  Synthetic Construct
IVGIVAGLAVLAVVVIGAVVATVMCRRKSSGGKGGSYSQAASSDSAQGSDVSLTA

SEQ ID NO: 31 - RNA, 317 nt, Artificial Sequence
  Synthetic Construct
cucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcu

SEQ ID NO: 32 - DNA, 311 bp, Artificial Sequence
  Synthetic Construct
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 33 - RNA, 136 nt, Artificial Sequence
  Synthetic Construct
cugguacugcaugcacgcaaugcuagcugccccuuucccguccuggguaccccgagucucccccgaccucgggucccagguaugcucccaccuccaccugccccacucaccaccucugcuaguuccagacaccucc

SEQ ID NO: 34 - DNA, 136 bp, Artificial Sequence
  Synthetic Construct
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 35 - RNA, 143 nt, Artificial Sequence
  Synthetic Construct
caagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccg

SEQ ID NO: 36 - DNA, 143 bp, Artificial Sequence
  Synthetic Construct
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccg

SEQ ID NO: 37 - RNA, 30 nt, Artificial Sequence
  Synthetic Construct
ggcggcucuggaggaggcggcuccggaggc

SEQ ID NO: 38 - DNA, 30 bp, Artificial Sequence
  Synthetic Construct
ggcggctctggaggaggcggctccggaggc

SEQ ID NO: 39 - protein, 10 aa, Artificial Sequence
  Synthetic Construct
GGSGGGGSGG

SEQ ID NO: 40 - DNA, 129 bp, Artificial Sequence
  Synthetic Construct
ggcgaactagtattcttctggtccccacagactcagagagaacccgccaccatgagagtgatggcccccagaaccctgatcctgctgctgtctggcgccctggccctgacagagacatgggccggaagc

SEQ ID NO: 41 - DNA, 488 bp, Artificial Sequence
  Synthetic Construct
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 42 - RNA, 740 nt, Artificial Sequence
  Synthetic Construct
  misc_feature 1,2: linked by (5′->5′)-pp(s)p- as shown in Table 5 and in Fig. 5 of the specification
  misc_feature 132: n = A,T,C,G or U
  misc_feature 132: Exists as polynucleotide sequence(s) as defined in the specification and may encode patient cancer-specific epitopes as defined in the specification (e.g., FIG. 3)
ggggcgaacuaguauucuucugguccccacagacucagagagaacccgccaccaugagagugauggcccccagaacccugauccugcugcugucuggcgcccuggcccugacagagacaugggccggaagcnaucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2021/0346485 A1
(43) Pub. Date: Nov. 11, 2021

(54) METHODS OF TREATING CANCER WITH A PD-1 AXIS BINDING ANTAGONIST AND AN RNA VACCINE

(71) Applicants: Genentech, Inc., South San Francisco, CA (US); BioNTech SE, Mainz (DE)

(72) Inventors: Lars MUELLER, South San Francisco, CA (US); Gregg Daniel FINE, South San Francisco, CA (US)

(73) Assignees: Genentech, Inc., South San Francisco, CA (US); BioNTech SE, Mainz (DE)

(21) Appl. No.: 17/373,175

(22) Filed: Jul. 12, 2021

Related U.S. Application Data
(63) Continuation of application No. PCT/US2020/013353, filed on Jan. 13, 2020.
(60) Provisional application No. 62/887,410, filed on Aug. 15, 2019.
(60) Provisional application No. 62/795,476, filed on Jan. 22, 2019.
(60) Provisional application No. 62/792,387, filed on Jan. 14, 2019.

(51) Int. Cl. A61K 39/00; A61K 39/395; A61K 9/127; A61K 47/24; A61K 47/18; A61P 35/00; C07K 14/74; C07K 14/47; C07H 21/02; C07H 21/04

(52) U.S. Cl. CPC A61K 39/001111; A61K 39/3955; A61K 9/127; A61K 47/24; A61K 47/186; C07K 2319/03; C07K 14/70539; C07K 14/4748; C07H 21/02; C07H 21/04; C07K 2319/02; A61P 35/00

(57) ABSTRACT

The present disclosure provides methods, uses, and kits for treating cancer in an individual. The methods comprise administering to the individual a PD-1 axis binding antagonist (such as an anti-PD-1 or anti-PD-L1 antibody) and an RNA vaccine (e.g., a personalized cancer vaccine that comprises one or more polynucleotides encoding one or more neoepitopes resulting from cancer-specific somatic mutations present in a tumor specimen obtained from the individual). Further provided herein are RNA molecules (e.g., a personalized RNA cancer vaccine that comprises one or more polynucleotides encoding one or more neoepitopes resulting from cancer-specific somatic mutations present in a tumor specimen obtained from the individual), as well as DNA molecules and methods useful for production or use of RNA vaccines.

### CROSS REFERENCE TO RELATED APPLICATIONS

This application claims the priority benefit of U.S. Provisional Application Ser. No. 62/792,387, filed Jan. 14, 2019; 62/795,476, filed Jan. 22, 2019; and 62/887,410, filed Aug. 15, 2019; each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392046940SEQLIST.txt, date recorded: Jan. 13, 2020, size: 41 KB).

### FIELD

The present disclosure relates to methods, uses, and kits related to treating cancers by administering a PD-1 axis binding antagonist (e.g., an anti-PD-1 or anti-PD-L1 antibody) in combination with an RNA vaccine. Further provided herein are RNA molecules (e.g., a personalized RNA cancer vaccine that comprises one or more polynucleotides encoding one or more neoepitopes resulting from cancer-specific somatic mutations present in a tumor specimen obtained from the individual), as well as DNA molecules and methods useful for production or use of RNA vaccines.

### BACKGROUND

Melanoma is a potentially deadly form of skin cancer originating from melanocytes. In 2012, there were approximately 232,000 new cases and 55,000 deaths from melanoma worldwide, with more than 100,000 new cases and 22,000 deaths in Europe (Ferlay J, Steliarova-Foucher E, Lortet-Tieulent J, et al. Eur J Cancer 2013; 49:1374-403). In the United States in 2018, an estimated 91,270 new diagnoses of melanoma are projected and approximately 9,320 patients are expected to die of the disease (American Cancer Society 2018). Additionally, estimates suggest a doubling of the incidence of melanoma every 10-20 years (Garbe C, Leiter U. Clin Dermatol 2009; 27:3-9).

The clinical outcome of patients with melanoma is highly dependent on the stage at presentation. Until recently, treatment options for metastatic melanoma were limited. Dacarbazine was considered to be the standard first-line treatment; however, outcomes were poor, with response rates of 5%-12%, median progression-free survival (PFS) of less than 2 months, and median overall survival (OS) of 6.4 to 9.1 months (Middleton M R, Grob J J, Aaronson N, et al. J Clin Oncol 2000; 18:158-66; Bedikian A Y, Millward M, Pehamberger H, et al. J Clin Oncol 2006; 24:4738-45; Chapman P B, Hauschild A, Robert C, et al. N Engl J Med 2011; 364:2507-16; Robert C, Thomas L, Bondarenko I, et al. N Engl J Med 2011; 364:2517-26). Combination chemotherapy and chemotherapy combined with interferon-α (IFN)-α or interleukin-2 (IL-2), although showing improved response rates, have not resulted in improved OS (Chapman P B, Einhorn L H, Meyers M L, et al. J Clin Oncol 1999; 17:2745-51; Ives N J, Stowe R L, Lorigan P, et al. J Clin Oncol 2007; 25:5426-34).

Immunotherapeutic agents that target co-inhibitory receptors or “immune checkpoints” that suppress T-cell activation have improved the outcomes of patients with advanced melanoma. Despite these advances, many patients do not respond to current therapies or later succumb to their disease, highlighting the continuing unmet medical need for more efficacious treatment options.

Clinical and nonclinical data on currently available immunotherapeutics suggest that single-agent immunotherapy is unlikely to induce complete and durable anti-tumor responses in the majority of patients. Host immunosuppression by malignant cells is mediated by multiple pathways; therefore, combination therapy regimens employing two or more targeted cancer immunotherapy (CIT) agents may be required to fully engage the anti-tumor potential of the host immune system.

Therapeutic vaccines, while promising, have historically fallen short of expectations. One of the potential reasons is that cancer-specific T cells become functionally exhausted during chronic exposure to cancer cells.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Provided herein are methods, kits, and uses involving a PD-1 axis binding antagonist (e.g., an anti-PD1 or anti-PD-L1 antibody) and an RNA vaccine for treating cancer.

In some aspects, provided herein are methods of treating cancer in an individual, comprising administering to the individual an effective amount of a PD-1 axis binding antagonist and an RNA vaccine, wherein the RNA vaccine comprises one or more polynucleotides encoding one or more neoepitopes resulting from cancer-specific somatic mutations present in a tumor specimen obtained from the individual.

In some embodiments, the PD-1 axis binding antagonist is a PD-1 binding antagonist. In some embodiments, the PD-1 binding antagonist is an anti-PD-1 antibody. In some embodiments, the anti-PD-1 antibody is nivolumab or pembrolizumab. In some embodiments, the anti-PD-1 antibody is administered to the individual at a dose of about 200 mg.

In some embodiments, the PD-1 axis binding antagonist is a PD-L1 binding antagonist. In some embodiments, the PD-L1 binding antagonist is an anti-PD-L1 antibody. In some embodiments, the anti-PD-L1 antibody is avelumab or durvalumab. In some embodiments, the anti-PD-L1 antibody comprises: (a) a heavy chain variable region (VH) that comprises an HVR-H1 comprising an amino acid sequence of GFTFSDSWIH (SEQ ID NO:1), an HVR-2 comprising an amino acid sequence of AWISPYGGSTYYADSVKG (SEQ ID NO:2), and HVR-3 comprising an amino acid RHWPGGFDY (SEQ ID NO:3), and (b) a light chain variable region (VL) that comprises an HVR-L1 comprising an amino acid sequence of RASQDVSTAVA (SEQ ID NO:4), an HVR-L2 comprising an amino acid sequence of SASFLYS (SEQ ID NO:5), and an HVR-L3 comprising an amino acid sequence of QQYLYHPAT (SEQ ID NO:6). In some embodiments, the anti-PD-L1 antibody comprises a heavy chain variable region (V_{H}) comprising an amino acid sequence of SEQ ID NO:7 and a light chain variable region (V_{L}) comprising an amino acid sequence of SEQ ID NO:8. In some embodiments, the anti-PD-L1 antibody is atezolizumab. In some embodiments, the anti-PD-L1 antibody is administered to the individual at a dose of about 1200 mg.

In some embodiments of any of the above embodiments, the PD-1 axis binding antagonist is administered to the individual at an interval of 21 days or 3 weeks.

In some embodiments, the RNA vaccine comprises one or more polynucleotides encoding 10-20 neoepitopes resulting from cancer-specific somatic mutations present in the tumor specimen. In some embodiments, the RNA vaccine is formulated in a lipoplex nanoparticle or liposome. In some embodiments, the RNA vaccine is administered to the individual at a dose of about 15 μg, about 25 μg, about 38 μg, about 50 μg, or about 100 μg. In some embodiments, the RNA vaccine is administered to the individual at an interval of 21 days or 3 weeks.

In some embodiments, the PD-1 axis binding antagonist and the RNA vaccine are administered to the individual in 8 21-day Cycles, and the RNA vaccine is administered to the individual on Days 1, 8, and 15 of Cycle 2 and Day 1 of Cycles 3-7. In some embodiments, the PD-1 axis binding antagonist is administered to the individual on Day 1 of Cycles 1-8. In some embodiments, the PD-1 axis binding antagonist and the RNA vaccine are further administered to the individual after Cycle 8. In some embodiments, the PD-1 axis binding antagonist and the RNA vaccine are further administered to the individual in 17 additional 21-day Cycles, the PD-1 axis binding antagonist is administered to the individual on Day 1 of Cycles 13-29, and the RNA vaccine is administered to the individual on Day 1 of Cycles 13, 21, and 29. In some embodiments, the PD-1 axis binding antagonist and the RNA vaccine are administered to the individual in 8 21-day Cycles, the PD-1 axis binding antagonist is pembrolizumab and is administered to the individual at a dose of about 200 mg on Day 1 of Cycles 1-8, and the RNA vaccine is administered to the individual at a dose of about 25 μg on Days 1, 8, and 15 of Cycle 2 and Day 1 of Cycles 3-7. In some embodiments, the RNA vaccine is administered to the individual at doses of about 25 μg on Day 1 of Cycle 2, about 25 μg on Day 8 of Cycle 2, about 25 μg on Day 15 of Cycle 2, and about 25 μg on Day 1 of each of Cycles 3-7. In some embodiments, the PD-1 axis binding antagonist and the RNA vaccine are administered intravenously. In some embodiments, the individual is a human.

In some embodiments, the cancer is selected from the group consisting of non-small cell lung cancer, bladder cancer, colorectal cancer, triple negative breast cancer, renal cancer, and head and neck cancer. In some embodiments, the cancer is melanoma. In some embodiments, the melanoma is cutaneous or mucosal melanoma. In some embodiments, the melanoma is not ocular or acral melanoma. In some embodiments, the melanoma is metastatic (e.g., stage IV, such as recurrent or de novo stage IV) or unresectable locally advanced (e.g., stage IIIC or stage IIID) melanoma. In some embodiments, the melanoma is previously untreated advanced melanoma. In some embodiments, the method results in improved progression-free survival (PFS). In some embodiments, the method results in increased objective response rate (ORR).

In some aspects, provided herein are kits or articles of manufacture comprising a PD-1 axis binding antagonist for use in combination with an RNA vaccine for treating an individual having cancer according to a method of any one of the above embodiments.

In some aspects, provided herein is a PD-1 axis binding antagonist for use in a method of treating a human individual having cancer, the method comprising administering to the individual an effective amount of the PD-1 axis binding antagonist in combination with an RNA vaccine, wherein the RNA vaccine comprises one or more polynucleotides encoding one or more neoepitopes resulting from cancer-specific somatic mutations present in a tumor specimen obtained from the individual. In some aspects, provided herein is an RNA vaccine for use in a method of treating a human individual having cancer, the method comprising administering to the individual an effective amount of the RNA vaccine in combination with a PD-1 axis binding antagonist, wherein the RNA vaccine comprises one or more polynucleotides encoding one or more neoepitopes resulting from cancer-specific somatic mutations present in a tumor specimen obtained from the individual.

In some aspects, provided herein is an RNA molecule comprising, in the 5′→3′ direction: (1) a 5′ cap; (2) a 5′ untranslated region (UTR); (3) a polynucleotide sequence encoding a secretory signal peptide; (4) a polynucleotide sequence encoding at least a portion of a transmembrane and cytoplasmic domain of a major histocompatibility complex (MHC) molecule; (5) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (6) a poly(A) sequence.

In some embodiments, the RNA molecule further comprises a polynucleotide sequence encoding at least one neoepitope; wherein the polynucleotide sequence encoding the at least one neoepitope is between the polynucleotide sequence encoding the secretory signal peptide (e.g., (3) above) and the polynucleotide sequence encoding the at least portion of the transmembrane and cytoplasmic domain of the MHC molecule (e.g., (4) above) in the 5′→3′ direction. In some embodiments, the RNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA molecule further comprises, in the 5′→3′ direction: a polynucleotide sequence encoding an amino acid linker; and a polynucleotide sequence encoding a neoepitope; wherein the polynucleotide sequences encoding the amino acid linker and the neoepitope form a first linker-neoepitope module; and wherein the polynucleotide sequences forming the first linker-neoepitope module are between the polynucleotide sequence encoding the secretory signal peptide (e.g., (3) above) and the polynucleotide sequence encoding the at least portion of the transmembrane and cytoplasmic domain of the MHC molecule (e.g., (4) above) in the 5′→3′ direction. In some embodiments, the amino acid linker comprises the sequence GGSGGGGSGG (SEQ ID NO:39). In some embodiments, the polynucleotide sequence encoding the amino acid linker comprises the sequence GGCGGCUCUGGAGGAGGCGGCUCCGGAGGC (SEQ ID NO:37). In some embodiments, the RNA molecule further comprises, in the 5′→3′ direction: at least a second linker-epitope module, wherein the at least second linker-epitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule (e.g., (4) above) in the 5′→3′ direction; and wherein the neoepitope of the first linker-epitope module is different from the neoepitope of the second linker-epitope module. In some embodiments, the RNA molecule comprises 2, 3, 4, 5, 6, 7, 8, 9, 10, 11, 12, 13, 14, 15, 16, 17, 18, 19, or 20 linker-epitope modules, and each of the linker-epitope modules encodes a different neoepitope. In some embodiments, the RNA molecule comprises 2, 3, 4, 5, 6, 7, 8, 9, 10, 11, 12, 13, 14, 15, 16, 17, 18, 19, or 20 linker-epitope modules, and the RNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule (e.g., (4) above). In some embodiments, the RNA molecule comprises the sequence shown in FIG. 4. In some embodiments, N in FIG. 4 represents a polynucleotide sequence encoding one or more neoepitopes (e.g., encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes). In some embodiments, N in FIG. 4 represents one or more (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different) linker-neoepitope module(s), each module comprising a polynucleotide sequence encoding one or more amino acid linker and a polynucleotide sequence encoding a neoepitope in the 5′→3′ direction.

In some embodiments, the 5′ cap (e.g., (1) above) of the RNA molecule comprises a D1 diastereoisomer of the structure:

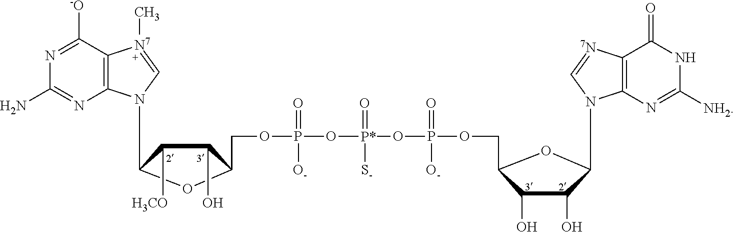

In some embodiments, the 5′ UTR (e.g., (2) above) of the RNA molecule comprises the sequence UUCUUCUGGUCCCCACAGACUCAGAGAGAACCCGCCACC (SEQ ID NO:23). In some embodiments, the 5′ UTR (e.g., (2) above) of the RNA molecule comprises the sequence GGCGAACUAGUAUUCUUCUGGUCCCCACAGACUCAGAGAGAACCCGCCACC (SEQ ID NO:21). In some embodiments, the secretory signal peptide (e.g., in (3) above) encoded by the RNA molecule comprises the amino acid sequence MRVMAPRTLILLLSGALALTETWAGS (SEQ ID NO:27). In some embodiments, the polynucleotide sequence encoding the secretory signal peptide (e.g., (3) above) of the RNA molecule comprises the sequence
AUGAGAGUGAUGGCCCCCAGAACCCUGAUCCUGCUGCUGUCUGGCGCCCUGGCCCUGACA GAGACAUGGGCCGGAAGC (SEQ ID NO:25). In some embodiments, the at least portion of the transmembrane and cytoplasmic domain of the MHC molecule (e.g., (4) above) encoded by the RNA molecule comprises the amino acid sequence
IVGIVAGLAVLAVVVIGAVVATVMCRRKSSGGKGGSYSQAASSDSAQGSDVSLTA (SEQ ID NO:30). In some embodiments, the polynucleotide sequence encoding the at least portion of the transmembrane and cytoplasmic domain of the MHC molecule (e.g., (4) above) of the RNA molecule comprises the sequence
AUCGUGGGAAUUGUGGCAGGACUGGCAGUGCUGGCCGUGGUGGUGAUCGGAGCCGUGGU GGCUACCGUGAUGUGCAGACGGAAGUCCAGCGGAGGCAAGGGCGGCAGCUACAGCCAGGC CGCCAGCUCUGAUAGCGCCCAGGGCAGCGACGUGUCACUGACAGCC (SEQ ID NO:28). In some embodiments, the 3′ untranslated region of the AES mRNA (e.g., (5a) above) of the RNA molecule comprises the sequence
CUGGUACUGCAUGCACGCAAUGCUAGCUGCCCCUUUCCCGUCCUGGGUACCCCGAGUCUC CCCCGACCUCGGGUCCCAGGUAUGCUCCCACCUCCACCUGCCCCACUCACCACCUCUGCUA GUUCCAGACACCUCC (SEQ ID NO:33). In some embodiments, wherein the non-coding RNA of the mitochondrially encoded 12S RNA (e.g., (5b) above) of the RNA molecule comprises the sequence
CAAGCACGCAGCAAUGCAGCUCAAAACGCUUAGCCUAGCCACACCCCCACGGGAAACAGC AGUGAUUAACCUUUAGCAAUAAACGAAAGUUUAACUAAGCUAUACUAACCCCAGGGUUG GUCAAUUUCGUGCCAGCCACACCG (SEQ ID NO:35). In some embodiments, the 3′ UTR (e.g., (5) above) of the RNA molecule comprises the sequence
CUCGAGCUGGUACUGCAUGCACGCAAUGCUAGCUGCCCCUUUCCCGUCCUGGGUACCCCG AGUCUCCCCCGACCUCGGGUCCCAGGUAUGCUCCCACCUCCACCUGCCCCACUCACCACCU CUGCUAGUUCCAGACACCUCCCAAGCACGCAGCAAUGCAGCUCAAAACGCUUAGCCUAGC CACACCCCCACGGGAAACAGCAGUGAUUAACCUUUAGCAAUAAACGAAAGUUUAACUAAG CUAUACUAACCCCAGGGUUGGUCAAUUUCGUGCCAGCCACACCGAGACCUGGUCCAGAGU CGCUAGCCGCGUCGCU (SEQ ID NO:31). In some embodiments, the poly(A) sequence (e.g., (6) above) of the RNA molecule comprises 120 adenine nucleotides.

In some aspects, provided herein is an RNA molecule comprising, in the 5′→3′ direction: the polynucleotide sequence

GGCGAACUAGUAUUCUUCUGGUCCCCACAGACUCAGAGAGAACCCGCCACCAUGAGAGUG AUGGCCCCCAGAACCCUGAUCCUGCUGCUGUCUGGCGCCCUGGCCCUGACAGAGACAUGG GCCGGAAGC (SEQ ID NO:19); and the polynucleotide sequence

(SEQ ID NO: 20)
AUCGUGGGAAUUGUGGCAGGACUGGCAGUGCUGGCCGUGGUGGUGAUCGG
AGCCGUGGUGGCUACCGUGAUGUGCAGACGGAAGUCCAGCGGAGGCAAGG
GCGGCAGCUACAGCCAGGCCGCCAGCUCUGAUAGCGCCCAGGGCAGCGAC
GUGUCACUGACAGCCUAGUAACUCGAGCUGGUACUGCAUGCACGCAAUGC
UAGCUGCCCCUUUCCCGUCCUGGGUACCCCGAGUCUCCCCCGACCUCGGG
UCCCAGGUAUGCUCCCACCUCCACCUGCCCCACUCACCACCUCUGCUAGU
UCCAGACACCUCCCAAGCACGCAGCAAUGCAGCUCAAAACGCUUAGCCUA
GCCACACCCCCACGGGAAACAGCAGUGAUUAACCUUUAGCAAUAAACGAA
AGUUUAACUAAGCUAUACUAACCCCAGGGUUGGUCAAUUUCGUGCCAGCC
ACACCGAGACCUGGUCCAGAGUCGCUAGCCGCGUCGCU.

In some aspects, provided herein is an RNA molecule comprising, in the 5′→3′ direction: the polynucleotide sequence

(SEQ ID NO: 42)
GGGGCGAACU AGUAUUCUUC UGGUCCCCAC AGACUCAGAG AGAACCCGCC
ACCAUGAGAG UGAUGGCCCC CAGAACCCUG AUCCUGCUGC UGUCUGGCGC
CCUGGCCCUG ACAGAGACAU GGGCCGGAAG CNAUCGUGGGA AUUGUGGCAG
GACUGGCAGU GCUGGCCGUG GUGGUGAUCG GAGCCGUGGU GGCUACCGUG
AUGUGCAGAC GGAAGUCCAG CGGAGGCAAG GGCGGCAGCU ACAGCCAGGC
CGCCAGCUCU GAUAGCGCCC AGGGCAGCGA CGUGUCACUG ACAGCCUAGU
AACUCGAGCU GGUACUGCAU GCACGCAAUG CUAGCUGCCC CUUUCCCGUC
CUGGGUACCC CGAGUCUCCC CCGACCUCGG GUCCCAGGUA UGCUCCCACC
UCCACCUGCC CCACUCACCA CCUCUGCUAG UUCCAGACAC CUCCCAAGCA
CGCAGCAAUG CAGCUCAAAA CGCUUAGCCU AGCCACACCC CCACGGGAAA
CAGCAGUGAU UAACCUUUAG CAAUAAACGA AAGUUUAACU AAGCUAUACU
AACCCCAGGG UUGGUCAAUU UCGUGCCAGC CACACCGAGA CCUGGUCCAG
AGUCGCUAGC CGCGUCGCUA AAAAAAAAAA AAAAAAAAAA AAAAAAAAAA
AAAAAAAAAA AAAAAAAAAA AAAAAAAAAA AAAAAAAAAA AAAAAAAAAA
AAAAAAAAAA AAAAAAAAAA AAAAAAAAAA AAAAAAAAA

In some embodiments, the RNA molecule further comprises a polynucleotide sequence encoding at least one neoepitope; wherein the polynucleotide sequence encoding the at least one neoepitope is between the sequences of SEQ ID NO:19 and SEQ ID NO:20, or at the position marked “N” in SEQ ID NO:42. In some embodiments, the RNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA molecule further comprises, in the 5′→3′ direction (e.g., between the sequences of SEQ ID NO:19 and SEQ ID NO:20, or at the position marked “N” in SEQ ID NO:42): (a) at least a first linker-neoepitope module, wherein the at least first linker-neoepitope module comprises a polynucleotide sequence encoding an amino acid linker and a polynucleotide sequence encoding a neoepitope; and (b) a second polynucleotide sequence encoding an amino acid linker. In some embodiments, the RNA molecule comprises 2, 3, 4, 5, 6, 7, 8, 9, 10, 11, 12, 13, 14, 15, 16, 17, 18, 19, or 20 linker-epitope modules, and each of the linker-epitope modules encodes a different neoepitope. In some embodiments, the RNA molecule comprises 2, 3, 4, 5, 6, 7, 8, 9, 10, 11, 12, 13, 14, 15, 16, 17, 18, 19, or 20 linker-epitope modules, and the RNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA molecule further comprises a 5′ cap, wherein the 5′ cap is located 5′ to the sequence

GGCGAACUAGUAUUCUUCUGGUCCCCACAGACUCAGAGAGAACCCGCCACCAUGAGAGUG AUGGCCCCCAGAACCCUGAUCCUGCUGCUGUCUGGCGCCCUGGCCCUGACAGAGACAUGG GCCGGAAGC (SEQ ID NO:19). In some embodiments, the 5′ cap is located between two guanine nucleotides. In some embodiments, the RNA molecule further comprises a 5′ cap, wherein the 5′ cap is located between the first 2 G bases in SEQ ID NO:42 (e.g., shown in FIG. 4). In some embodiments, the 5′ cap comprises a D1 diastereoisomer of the structure:

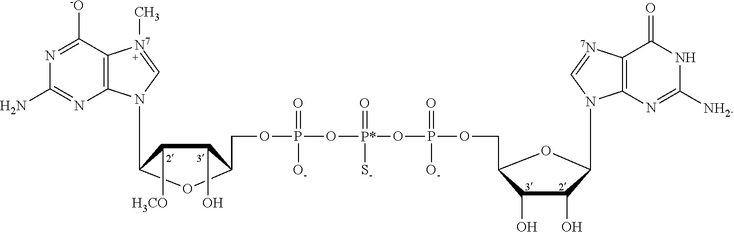

In some aspects, provided herein is a liposome comprising the RNA molecule of any one of the above embodiments (including, e.g., any of the RNA molecules described herein, or described in the Sequence listing or Figures) and one or more lipids, wherein the one or more lipids form a multilamellar structure that encapsulates the RNA molecule. In some embodiments, the one or more lipids comprises at least one cationic lipid and at least one helper lipid. In some embodiments, the one or more lipids comprises (R)—N,N,N-trimethyl-2,3-dioleyloxy-1-propanaminium chloride (DOTMA) and 1,2-dioleoyl-sn-glycero-3-phosphoethanolamine (DOPE). In some embodiments, at physiological pH the overall charge ratio of positive charges to negative charges of the liposome is 1.3:2 (0.65). In some embodiments, at physiological pH the overall charge ratio of positive charges to negative charges of the liposome is not lower than 1.0:2.0. In some embodiments, at physiological pH the overall charge ratio of positive charges to negative charges of the liposome is not higher than 1.9:2.0. In some embodiments, at physiological pH the overall charge ratio of positive charges to negative charges of the liposome is not lower than 1.0:2.0 and not higher than 1.9:2.0.

In some aspects, provided herein is a method of treating or delaying progression of cancer in an individual, comprising administering to the individual an effective amount of the RNA molecule of any one of the above embodiments (including, e.g., any of the RNA molecules described herein, or described in the Sequence listing or Figures) or the liposome of any one of the above embodiments. Also provided herein is the RNA molecule of any one of the above embodiments or the liposome of any one of the above embodiments for use in a method of treating or delaying progression of cancer in an individual, wherein the method comprises administering to the individual an effective amount of the RNA molecule or liposome. Also provided herein is the RNA molecule of any one of the above embodiments (including, e.g., any of the RNA molecules described herein, or described in the Sequence listing or Figures) or the liposome of any one of the above embodiments for use in the manufacture of a medicament for treating or delaying progression of cancer in an individual. In some embodiments, the RNA molecule comprises one or more polynucleotides encoding one or more neoepitopes resulting from cancer-specific somatic mutations present in a tumor specimen obtained from the individual. In some embodiments, the methods further comprise administering a PD-1 axis binding antagonist to the individual (e.g., an anti-PDL1 antibody). In some embodiments, the cancer is selected from the group consisting of melanoma, non-small cell lung cancer, bladder cancer, colorectal cancer, triple negative breast cancer, renal cancer, and head and neck cancer. In some embodiments, the RNA molecule or liposome is administered at a dose of about 15 μg, about 25 μg, about 38 μg, about 50 μg, or about 100 μg. In some embodiments, the RNA molecule or liposome is administered at a dose of about 15 μg, about 25 μg, about 38 μg, about 50 μg, or about 100 μg and the PD-1 axis binding antagonist (e.g., an anti-PDL1 antibody) is administered at a dose of about 200 or about 1200 mg. In some embodiments, the PD-1 axis binding antagonist and the RNA molecule or liposome are administered to the individual in 8 21-day Cycles, wherein the PD-1 axis binding antagonist is pembrolizumab and is administered to the individual at a dose of about 200 mg on Day 1 of Cycles 1-8, and wherein the RNA vaccine is administered to the individual at a dose of about 25 μg on Days 1, 8, and 15 of Cycle 2 and Day 1 of Cycles 3-7.

In some aspects, provided herein is a DNA molecule encoding any of the RNA molecules described herein. In some aspects, provided herein is a DNA molecule comprising, in the 5′→3′ direction: (1) a polynucleotide sequence encoding a 5′ untranslated region (UTR); (2) a polynucleotide sequence encoding a secretory signal peptide; (3) a polynucleotide sequence encoding at least a portion of a transmembrane and cytoplasmic domain of a major histocompatibility complex (MHC) molecule; (4) a polynucleotide sequence encoding a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (5) a polynucleotide sequence encoding a poly(A) sequence.

In some embodiments, the DNA molecule further comprises a polynucleotide sequence encoding at least one neoepitopes; wherein the polynucleotide sequence encoding the at least one neoepitope is between the polynucleotide sequence encoding the secretory signal peptide (e.g., (2) above) and the polynucleotide sequence encoding the at least portion of the transmembrane and cytoplasmic domain of the MHC molecule (e.g., (3) above) in the 5′→3′ direction. In some embodiments, the DNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the DNA molecule further comprises, in the 5′→3′ direction: a polynucleotide sequence encoding an amino acid linker; and a polynucleotide sequence encoding a neoepitope; wherein the polynucleotide sequences encoding the amino acid linker and the neoepitope form a first linker-neoepitope module; and wherein the polynucleotide sequences forming the first linker-neoepitope module are between the polynucleotide sequence encoding the secretory signal peptide (e.g., (2) above) and the polynucleotide sequence encoding the at least portion of the transmembrane and cytoplasmic domain of the MHC molecule (e.g., (3) above) in the 5′→3′ direction. In some embodiments, the amino acid linker comprises the sequence GGSGGGGSGG (SEQ ID NO:39). In some embodiments, the polynucleotide sequence encoding the amino acid linker comprises the sequence GGCGGCTCTGGAGGAGGCGGCTCCGGAGGC (SEQ ID NO:38). In some embodiments, the DNA molecule further comprises, in the 5′→3′ direction: at least a second linker-epitope module, wherein the at least second linker-epitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule (e.g., (3) above) in the 5′→3′ direction; and wherein the neoepitope of the first linker-epitope module is different from the neoepitope of the second linker-epitope module. In some embodiments, the DNA molecule comprises 2, 3, 4, 5, 6, 7, 8, 9, 10, 11, 12, 13, 14, 15, 16, 17, 18, 19, or 20 linker-epitope modules, and each of the linker-epitope modules encodes a different neoepitope. In some embodiments, the DNA molecule comprises 2, 3, 4, 5, 6, 7, 8, 9, 10, 11, 12, 13, 14, 15, 16, 17, 18, 19, or 20 linker-epitope modules, and the DNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the DNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule (e.g., (3) above). In some embodiments, the polynucleotide encoding the 5′ UTR (e.g., (1) above) comprises the sequence

TTCTTCTGGTCCCCACAGACTCAGAGAGAACCCGCCACC (SEQ ID NO:24). In some embodiments, the polynucleotide encoding the 5′ UTR (e.g., (1) above) comprises the sequence

(SEQ ID NO: 22)
GGCGAACTAGTATTCTTCTGGTCCCCACAGACTCAGAGAGAACCCGCCAC
C.

In some embodiments, the secretory signal peptide (e.g., encoded by (2) above) comprises the amino acid sequence MRVMAPRTLILLLSGALALTETWAGS (SEQ ID NO:27). In some embodiments, the polynucleotide sequence encoding the secretory signal peptide (e.g., (2) above) comprises the sequence ATGAGAGTGATGGCCCCCAGAACCCTGATCCTGCTGCTGTCTGGCGCCCTGGCCCTGACAGA GACATGGGCCGGAAGC (SEQ ID NO:26). In some embodiments, the at least portion of the transmembrane and cytoplasmic domain of the MHC molecule (e.g., encoded by (3) above) comprises the amino acid sequence
IVGIVAGLAVLAVVVIGAVVATVMCRRKSSGGKGGSYSQAASSDSAQGSDVSLTA (SEQ ID NO:30). In some embodiments, the polynucleotide sequence encoding the at least portion of the transmembrane and cytoplasmic domain of the MHC molecule (e.g., (3) above) comprises the sequence ATCGTGGGAATTGTGGCAGGACTGGCAGTGCTGGCCGTGGTGGTGATCGGAGCCGTGGTGG CTACCGTGATGTGCAGACGGAAGTCCAGCGGAGGCAAGGGCGGCAGCTACAGCCAGGCCGC CAGCTCTGATAGCGCCCAGGGCAGCGACGTGTCACTGACAGCC (SEQ ID NO:29). In some embodiments, the polynucleotide sequence encoding the 3′ untranslated region of the AES mRNA (e.g., (4a) above) comprises the sequence
CTGGTACTGCATGCACGCAATGCTAGCTGCCCCTTTCCCGTCCTGGGTACCCCGAGTCTCCCC CGACCTCGGGTCCCAGGTATGCTCCCACCTCCACCTGCCCCACTCACCACCTCTGCTAGTTCC AGACACCTCC (SEQ ID NO:34). In some embodiments, the polynucleotide encoding the non-coding RNA of the mitochondrially encoded 12S RNA (e.g., (4b) above) comprises the sequence
CAAGCACGCAGCAATGCAGCTCAAAACGCTTAGCCTAGCCACACCCCCACGGGAAACAGCA GTGATTAACCTTTAGCAATAAACGAAAGTTTAACTAAGCTATACTAACCCCAGGGTTGGTCA ATTTCGTGCCAGCCACACCG (SEQ ID NO:36). In some embodiments, the polynucleotide encoding the 3′ UTR (e.g., (4) above) comprises the sequence
CTGGTACTGCATGCACGCAATGCTAGCTGCCCCTTTCCCGTCCTGGGTACCCCGAGTCTCCCC CGACCTCGGGTCCCAGGTATGCTCCCACCTCCACCTGCCCCACTCACCACCTCTGCTAGTTCC AGACACCTCCCAAGCACGCAGCAATGCAGCTCAAAACGCTTAGCCTAGCCACACCCCCACG GGAAACAGCAGTGATTAACCTTTAGCAATAAACGAAAGTTTAACTAAGCTATACTAACCCCA GGGTTGGTCAATTTCGTGCCAGCCACACCGAGACCTGGTCCAGAGTCGCTAGCCGCGTCGCT (SEQ ID NO:32). In some embodiments, the poly(A) sequence (e.g., (5) above) comprises 120 adenine nucleotides.

In some aspects, provided herein is a DNA molecule comprising, in the 5′→3′ direction: the polynucleotide sequence

GGCGAACTAGTATTCTTCTGGTCCCCACAGACTCAGAGAGAACCCGCCACCATGAGAGTGAT GGCCCCCAGAACCCTGATCCTGCTGCTGTCTGGCGCCCTGGCCCTGACAGAGACATGGGCCG GAAGC (SEQ ID NO:40); and the polynucleotide sequence

(SEQ ID NO: 41)
ATCGTGGGAATTGTGGCAGGACTGGCAGTGCTGGCCGTGGTGGTGATCGG
AGCCGTGGTGGCTACCGTGATGTGCAGACGGAAGTCCAGCGGAGGCAAGG
GCGGCAGCTACAGCCAGGCCGCCAGCTCTGATAGCGCCCAGGGCAGCGAC
GTGTCACTGACAGCCTAGTAACTCGAGCTGGTACTGCATGCACGCAATGC
TAGCTGCCCCTTTCCCGTCCTGGGTACCCCGAGTCTCCCCCGACCTCGGG
TCCCAGGTATGCTCCCACCTCCACCTGCCCCACTCACCACCTCTGCTAGT
TCCAGACACCTCCCAAGCACGCAGCAATGCAGCTCAAAACGCTTAGCCTA
GCCACACCCCCACGGGAAACAGCAGTGATTAACCTTTAGCAATAAACGAA
AGTTTAACTAAGCTATACTAACCCCAGGGTTGGTCAATTTCGTGCCAGCC
ACACCGAGACCTGGTCCAGAGTCGCTAGCCGCGTCGCT.

In some embodiments, the DNA molecule further comprises a polynucleotide sequence encoding at least one neoepitope; wherein the polynucleotide sequence encoding the at least one neoepitope is between the sequences of SEQ ID NO:40 and SEQ ID NO:41. In some embodiments, the DNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the DNA molecule comprises, in the 5′→3′ direction between the sequences of SEQ ID NO:40 and SEQ ID NO:41: (a) at least a first linker-neoepitope module, wherein the at least first linker-neoepitope module comprises a polynucleotide sequence encoding an amino acid linker and a polynucleotide sequence encoding a neoepitope; and (b) a second polynucleotide sequence encoding an amino acid linker. In some embodiments, the DNA molecule comprises 2, 3, 4, 5, 6, 7, 8, 9, 10, 11, 12, 13, 14, 15, 16, 17, 18, 19, or 20 linker-epitope modules, and each of the linker-epitope modules encodes a different neoepitope. In some embodiments, the DNA molecule comprises 2, 3, 4, 5, 6, 7, 8, 9, 10, 11, 12, 13, 14, 15, 16, 17, 18, 19, or 20 linker-epitope modules, and the DNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes.

In some aspects, provided herein is a method of producing an RNA molecule, comprising transcribing the DNA molecule of any one of the above embodiments.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows the study schema for a Phase II, randomized, open-label study designed to evaluate the efficacy and safety of an RNA-based personalized cancer vaccine (RO7198457) plus anti-PD1 antibody (pembrolizumab). In the randomized stage, patients are randomized (2:1) to experimental treatment (Arm B) or control treatment (Arm A). IMC=internal monitoring committee; LDH=lactate dehydrogenase; Q3W=every 3 weeks; TBD=to be determined; ULN=upper limit of normal.

FIG. 2 shows the dosing schemas for Arm A (pembrolizumab) and for safety run-in stage and Arm B (RO7198457 plus pembrolizumab) of the Phase II study. C=cycle; D=day.

FIG. 3 shows the general structure of an exemplary RNA vaccine (i.e., a poly-neoepitope RNA). This figure is a schematic illustration of the general structure of the RNA drug substance with constant 5′-cap (beta-S-ARCA (D1)), 5′- and 3′-untranslated regions (hAg-Kozak and FI, respectively), N- and C-terminal fusion tags (sec_{2.0} and MITD, respectively), and poly(A)-tail (A120) as well as patient-specific sequences encoding the neoepitopes (neo1 to 10) fused by GS-rich linkers.

FIG. 4 is the ribonucleotide sequence (5′->3′) of the constant region of an exemplary RNA vaccine (SEQ ID NO:42). The linkage between the first two G residues is the unusual bond (5′→5′)-ppₛp- as shown in Table 5 and in FIG. 5 for the 5′ capping structure. The insertion site for patient cancer-specific sequences is between the C131 and A132 residues (marked in bold text). “N” refers to the position of polynucleotide sequence(s) encoding one or more (e.g., 1-20) neoepitopes (separated by optional linkers).

FIG. 5 is the 5′-capping structure beta-S-ARCA(D1) (m₂⁷.^{2′}.^{O}GppₛpG) used at the 5′ end of the RNA constant regions. The stereogenic P center is Rp-configured in the “D1” isomer. Note: Shown in red are the differences between beta-S-ARCA(D1) and the basic cap structure m⁷GppₛpG; an —OCH3 group at the C2′ position of the building block m⁷G and substitution of a non-bridging oxygen at the beta-phosphate by sulphur. Owing to the presence of a stereogenic P center (labelled with *), the phosphorothioate cap analogue beta-S-ARCA exists as two diastereomers. Based on their elution order in reversed-phase high-performance liquid chromatography, these have been designated as 01 and 02.

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e g, enhancing effector responses to antigen recognition). In some embodiments, the PD-1 binding antagonist is an anti-PD-1 antibody. Specific examples of PD-1 binding antagonists are provided infra.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e g, enhancing effector responses to antigen recognition). In some embodiments, a PD-L1 binding antagonist is an anti-PD-L1 antibody. Specific examples of PD-L1 binding antagonists are provided infra.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” or “in combination with” refers to administration of one treatment modality in addition to another treatment modality. As such, “in conjunction with” or “in combination with” refers to administration of one treatment modality before, during, or after administration of the other treatment modality to the individual.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the V_{H} and V_{L} domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10−7 M, preferably no more than about 1×10−8 M and preferably no more than about 1×10−9 M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat Numbering) |
| H1 | H31-H35 | H26-H35 | H26-H32 | H30-H35 (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The expression “linear antibodies” refers to the antibodies described in Zapata et al. (1995 Protein Eng, 8(10):1057-1062). Briefly, these antibodies comprise a pair of tandem Fd segments (VH—CH1-VH—CH1) which, together with complementary light chain polypeptides, form a pair of antigen binding regions. Linear antibodies can be bispecific or monospecific.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some embodiments, the sample is a sample obtained from the cancer of an individual (e.g., a tumor sample) that comprises tumor cells and, optionally, tumor-infiltrating immune cells. For example, the sample can be a tumor specimen that is embedded in a paraffin block, or that includes freshly cut, serial unstained sections. In some embodiments, the sample is from a biopsy and includes 50 or more viable tumor cells (e.g., from a core-needle biopsy and optionally embedded in a paraffin block; excisional, incisional, punch, or forceps biopsy; or a tumor tissue resection).

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

A cancer or biological sample which “has human effector cells” is one which, in a diagnostic test, has human effector cells present in the sample (e.g., infiltrating human effector cells).

A cancer or biological sample which “has FcR-expressing cells” is one which, in a diagnostic test, has FcR-expressing present in the sample (e.g., infiltrating FcR-expressing cells). In some embodiments, FcR is FcγR. In some embodiments, FcR is an activating FcγR.

### II. Overview

Provided herein is a method for treating or delaying progression of cancer in an individual comprising administering to the individual an effective amount of a PD-1 axis binding antagonist (e.g., an anti-PD-1 or anti-PD-L1 antibody) and an RNA vaccine. In some embodiments, the RNA vaccine comprises one or more polynucleotides encoding one or more neoepitopes resulting from cancer-specific somatic mutations present in the cancer, e.g., present in a tumor specimen obtained from the individual. In some embodiments, the individual is a human.

In some embodiments, provided herein is a method or treating or delaying progression of cancer in an individual comprising administering to the individual an effective amount of a PD-1 axis binding antagonist (e.g., an anti-PD-1 or anti-PD-L1 antibody) and an RNA vaccine, wherein the RNA vaccine comprises one or more polynucleotides encoding one or more neoepitopes identified based upon somatic mutations present in a tumor sample obtained from the individual. In some embodiments, provided herein is a method or treating or delaying progression of cancer in an individual comprising administering to the individual an effective amount of a PD-1 axis binding antagonist (e.g., an anti-PD-1 or anti-PD-L1 antibody) and an RNA vaccine, wherein the RNA vaccine comprises one or more polynucleotides encoding one or more neoepitopes corresponding to somatic mutations present in a tumor sample obtained from the individual.

In some embodiments, the treatment extends the progression free survival (PFS) and/or the overall survival (OS) of the individual, as compared to a treatment comprising administration of a PD-1 axis binding antagonist in the absence of an RNA vaccine. In some embodiments, the treatment improves overall response rate (ORR), as compared to a treatment comprising administration of a PD-1 axis binding antagonist in the absence of an RNA vaccine. In some embodiments, ORR refers to the proportion of patients with a complete response (CR) or partial response (PR). In some embodiments, the treatment extends the duration of response (DOR) in the individual, as compared to a treatment comprising administration of a PD-1 axis binding antagonist in the absence of an RNA vaccine. In some embodiments, the treatment improves health-related quality of life (HRQoL) score in the individual, as compared to a treatment comprising administration of a PD-1 axis binding antagonist in the absence of an RNA vaccine.

In some embodiments, the PD-1 axis binding antagonist is administered to the individual at an interval of 21 days or 3 weeks. In some embodiments, the PD-1 axis binding antagonist is an anti-PD-1 antibody (e.g., pembrolizumab) administered to the individual at an interval of 21 days or 3 weeks, e.g., at a dose of about 200 mg. In some embodiments, the PD-1 axis binding antagonist is an anti-PD-L1 antibody (e.g., atezolizumab) administered to the individual at an interval of 21 days or 3 weeks, e.g., at a dose of about 1200 mg.

In some embodiments, the RNA vaccine is administered to the individual at an interval of 21 days or 3 weeks.

In some embodiments, the PD-1 axis binding antagonist and the RNA vaccine are administered to the individual in 8 21-day Cycles. In some embodiments, the RNA vaccine is administered to the individual on Days 1, 8, and 15 of Cycle 2 and Day 1 of Cycles 3-7. In some embodiments, the PD-1 axis binding antagonist is administered to the individual on Day 1 of Cycles 1-8. In some embodiments, the RNA vaccine is administered to the individual on Days 1, 8, and 15 of Cycle 2 and Day 1 of Cycles 3-7, and the PD-1 axis binding antagonist is administered to the individual on Day 1 of Cycles 1-8.

In some embodiments, the PD-1 axis binding antagonist and the RNA vaccine are further administered to the individual after Cycle 8. In some embodiments, the PD-1 axis binding antagonist and the RNA vaccine are further administered to the individual in 17 additional 21-day Cycles, wherein the PD-1 axis binding antagonist is administered to the individual on Day 1 of Cycles 13-29, and/or wherein the RNA vaccine is administered to the individual on Day 1 of Cycles 13, 21, and 29.

In certain embodiments, a PD-1 axis binding antagonist and an RNA vaccine are administered to the individual in 8 21-day Cycles, wherein the PD-1 axis binding antagonist is pembrolizumab and is administered to the individual at a dose of about 200 mg on Day 1 of Cycles 1-8, and wherein the RNA vaccine is administered to the individual at a dose of about 25 μg on Days 1, 8, and 15 of Cycle 2 and Day 1 of Cycles 3-7. In certain embodiments, a PD-L1 axis binding antagonist and the RNA vaccine are administered to the individual in 8 21-day Cycles, wherein the PD-L1 axis binding antagonist is atezolizumab and is administered to the individual at a dose of about 1200 mg on Day 1 of Cycles 1-8, and wherein the RNA vaccine is administered to the individual at a dose of about 25 μg on Days 1, 8, and 15 of Cycle 2 and Day 1 of Cycles 3-7. In some embodiments, the RNA vaccine is administered to the individual at doses of about 25 μg on Day 1 of Cycle 2, about 25 μg on Day 8 of Cycle 2, about 25 μg on Day 15 of Cycle 2, and about 25 μg on Day 1 of each of Cycles 3-7 (that is to say, a total of about 75 μg of the vaccine is administered to the individual over 3 doses during Cycle 2). In some embodiments, a total of about 75 μg of the vaccine is administered to the individual over 3 doses during the first Cycle in which the RNA vaccine is administered. In some embodiments, the PCV is administered intravenously, for example, in a liposomal formulation, at doses of 15 μg, 25 μg, 38 μg, 50 μg, or 100 μg. In some embodiments, 15 μg, 25 μg, 38 μg, 50 μg, or 100 μg of RNA is delivered per dose (i.e., dose weight reflects the weight of RNA administered, not the total weight of the formulation or lipoplex administered).

### III. RNA Vaccines

Certain aspects of the present disclosure relate to personalized cancer vaccines (PCVs). In some embodiments, the PCV is an RNA vaccine. Features of exemplary RNA vaccines are described infra. In some embodiments, the present disclosure provides an RNA polynucleotide comprising one or more of the features/sequences of the RNA vaccines described infra. In some embodiments, the RNA polynucleotide is a single-stranded mRNA polynucleotide. In other embodiments, the present disclosure provides a DNA polynucleotide encoding an RNA comprising one or more of the features/sequences of the RNA vaccines described infra.

Personalized cancer vaccines comprise individualized neoantigens (i.e., tumor-associated antigens (TAAs) that are specifically expressed in the patient's cancer) identified as having potential immunostimulatory activities. In the embodiments described herein, the PCV is a nucleic acid, e.g., messenger RNA. Accordingly, without wishing to be bound by theory, it is believed that upon administration, the personalized cancer vaccine is taken up and translated by antigen presenting cells (APCs) and the expressed protein is presented via major histocompatibility complex (MHC) molecules on the surface of the APCs. This leads to an induction of both cytotoxic T-lymphocyte (CTL)- and memory T-cell-dependent immune responses against cancer cells expressing the TAA(s).

PCVs typically include multiple neoantigen epitopes (“neoepitopes”), e.g., 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 neoepitopes or at least 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 neoepitopes, optionally with linker sequences between the individual neoepitopes. In some embodiments, a neoepitope as used herein refers to a novel epitope that is specific for a patient's cancer but not found in normal cells of the patient. In some embodiments, the neoepitope is presented to T cells when bound to MHC. In some embodiments, the PCV also includes a 5′ mRNA cap analogue, a 5′ UTR, a signal sequence, a domain to facilitate antigen expression, a 3′ UTR, and/or a polyA tail. In some embodiments, the RNA vaccine comprises one or more polynucleotides encoding 10-20 neoepitopes resulting from cancer-specific somatic mutations present in the tumor specimen. In some embodiments, the RNA vaccine comprises one or more polynucleotides encoding at least 5 neoepitopes resulting from cancer-specific somatic mutations present in the tumor specimen. In some embodiments, the RNA vaccine comprises one or more polynucleotides encoding 5-20 neoepitopes resulting from cancer-specific somatic mutations present in the tumor specimen. In some embodiments, the RNA vaccine comprises one or more polynucleotides encoding 5-10 neoepitopes resulting from cancer-specific somatic mutations present in the tumor specimen.

In some embodiments, the manufacture of an RNA vaccine of the present disclosure is a multi-step process, whereby somatic mutations in the patient's tumor are identified by next-generation sequencing (NGS) and immunogenic neoantigen epitopes (or “neoepitopes”) are predicted. The RNA cancer vaccine targeting the selected neoepitopes is manufactured on a per-patient basis. In some embodiments, the vaccine is an RNA-based cancer vaccine consisting of up to two messenger RNA molecules, each encoding up to 10 neoepitopes (for a total of up to 20 neoepitopes), which are specific to the patient's tumor.

In some embodiments, expressed non-synonymous mutations are identified by whole exome sequencing (WES) of tumor DNA and peripheral blood mononuclear cell (PBMC) DNA (as a source of healthy tissue from the patient) as well as tumor RNA sequencing (to assess expression). From the resulting list of mutant proteins, potential neoantigens are predicted using a bioinformatics workflow that ranks their likely immunogenicity on the basis of multiple factors, including the binding affinity of the predicted epitope to individual major histocompatibility complex (MHC) molecules, and the level of expression of the associated RNA. The mutation discovery, prioritization, and confirmation processes are complemented by a database that provides comprehensive information about expression levels of respective wild-type genes in healthy tissues. This information enables the development of a personalized risk mitigation strategy by removing target candidates with an unfavorable risk profile. Mutations occurring in proteins with a possible higher auto-immunity risk in critical organs are filtered out and not considered for vaccine production. In some embodiments, up to 20 MHCI and MHCII neoepitopes that are predicted to elicit CD8⁺ T-cell and/or CD4⁺ T-cell responses, respectively, for an individual patient are selected for inclusion into the vaccine. Vaccinating against multiple neoepitopes is expected to increase the breadth and magnitude of the overall immune response to PCV and may help to mitigate the risk of immune escape, which can occur when tumors are exposed to the selective pressure of an effective immune response (Tran E, Robbins P F, Lu Y C, et al. N Engl J Med 2016; 375:2255-62; Verdegaal E M, de Miranda N F, Visser M, et al. Nature 2016; 536:91-5).

In some embodiments, the RNA vaccine comprises one or more polynucleotide sequences encoding an amino acid linker. For example, amino acid linkers can be used between 2 patient-specific neoepitope sequences, between a patient-specific neoepitope sequence and a fusion protein tag (e.g., comprising sequence derived from an MHC complex polypeptide), or between a secretory signal peptide and a patient-specific neoepitope sequence. In some embodiments, the RNA vaccine encodes multiple linkers. In some embodiments, the RNA vaccine comprises one or more polynucleotides encoding 5-20 neoepitopes resulting from cancer-specific somatic mutations present in the tumor specimen, and the polynucleotides encoding each epitope are separated by a polynucleotide encoding a linker sequence. In some embodiments, the RNA vaccine comprises one or more polynucleotides encoding 5-10 neoepitopes resulting from cancer-specific somatic mutations present in the tumor specimen, and the polynucleotides encoding each epitope are separated by a polynucleotide encoding a linker sequence. In some embodiments, polynucleotides encoding linker sequences are also present between the polynucleotides encoding an N-terminal fusion tag (e.g., a secretory signal peptide) and a polynucleotide encoding one of the neoepitopes and/or between a polynucleotide encoding one of the neoepitopes and the polynucleotides encoding a C-terminal fusion tag (e.g., comprising a portion of an MHC polypeptide). In some embodiments, two or more linkers encoded by the RNA vaccine comprise different sequences. In some embodiments, the RNA vaccine encodes multiple linkers, all of which share the same amino acid sequence.

A variety of linker sequences are known in the art. In some embodiments, the linker is a flexible linker. In some embodiments, the linker comprises G, S, A, and/or T residues. In some embodiments, the linker consists of glycine and serine residues. In some embodiments, the linker is between about 5 and about 20 amino acids or between about 5 and about 12 amino acids in length, e.g., about 5, about 6, about 7, about 8, about 9, about 10, about 11, about 12, about 13, about 14, about 15, about 16, about 17, about 18, about 19, or about 20 amino acids in length. In some embodiments, the linker comprises the sequence GGSGGGGSGG (SEQ ID NO:39). In some embodiments, the linker of the RNA vaccine comprises the sequence GGCGGCUCUGGAGGAGGCGGCUCCGGAGGC (SEQ ID NO:37). In some embodiments, the linker of the RNA vaccine is encoded by DNA comprising the sequence

(SEQ ID NO: 38)
GGCGGCTCTGGAGGAGGCGGCTCCGGAGGC.

In some embodiments, the RNA vaccine comprises a 5′ cap. The basic mRNA cap structure is known to contain a 5′-5′ triphosphate linkage between 2 nucleosides (e.g., two guanines) and a 7-methyl group on the distal guanine, i.e., m⁷GppₛpG. Exemplary cap structures can be found, e.g., in U.S. Pat. Nos. 8,153,773 and 9,295,717 and Kuhn, A. N. et al. (2010) Gene Ther. 17:961-971. In some embodiments, the 5′ cap has the structure m2⁷.^{2′}.^{O}GppₛpG. In some embodiments, the 5′ cap is a beta-S-ARCA cap. The S-ARCA cap structure includes a 2′-O methyl substitution (e.g., at the C2′ position of the m⁷G) and an S-substitution at one or more of the phosphate groups. In some embodiments, the 5′ cap comprises the structure:

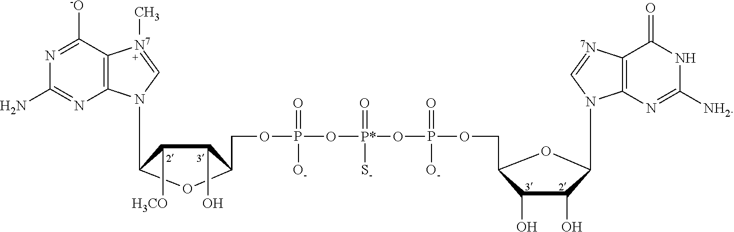

In some embodiments, the 5′ cap is the D1 diastereoisomer of beta-S-ARCA (see, e.g., U.S. Pat. No. 9,295,717). The * in the above structure indicates a stereogenic P center, which can exist in two diastereoisomers (designated D1 and D2). The D1 diastereomer of beta-S-ARCA or beta-S-ARCA(D1) is the diastereomer of beta-S-ARCA which elutes first on an HPLC column compared to the D2 diastereomer of beta-S-ARCA (beta-S-ARCA(D2)) and thus exhibits a shorter retention time. The HPLC preferably is an analytical HPLC. In one embodiment, a Supelcosil LC-18-T RP column, preferably of the format: 5 μm, 4.6×250 mm is used for separation, whereby a flow rate of 1.3 ml/min can be applied. In one embodiment, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

In some embodiments, the RNA vaccine comprises a 5′ UTR. Certain untranslated sequences found 5′ to protein-coding sequences in mRNAs have been shown to increase translational efficiency. See, e.g., Kozak, M. (1987) J. Mol. Biol. 196:947-950. In some embodiments, the 5′ UTR comprises sequence from the human alpha globin mRNA. In some embodiments, the RNA vaccine comprises a 5′ UTR sequence of UUCUUCUGGUCCCCACAGACUCAGAGAGAACCCGCCACC (SEQ ID NO:23). In some embodiments, the 5′ UTR sequence of the RNA vaccine is encoded by DNA comprising the sequence TTCTTCTGGTCCCCACAGACTCAGAGAGAACCCGCCACC (SEQ ID NO:24). In some embodiments, the 5′ UTR sequence of RNA vaccine comprises the sequence GGCGAACUAGUAUUCUUCUGGUCCCCACAGACUCAGAGAGAACCCGCCACC (SEQ ID NO:21). In some embodiments, the 5′ UTR sequence of RNA vaccine is encoded by DNA comprising the sequence GGCGAACTAGTATTCTTCTGGTCCCCACAGACTCAGAGAGAACCCGCCACC (SEQ ID NO:22).

In some embodiments, the RNA vaccine comprises a polynucleotide sequence encoding a secretory signal peptide. As is known in the art, a secretory signal peptide is an amino acid sequence that directs a polypeptide to be trafficked from the endoplasmic reticulum and into the secretory pathway upon translation. In some embodiments, the signal peptide is derived from a human polypeptide, such as an MHC polypeptide. See, e.g., Kreiter, S. et al. (2008) J. Immunol. 180:309-318, which describes an exemplary secretory signal peptide that improves processing and presentation of MHC Class I and II epitopes in human dendritic cells. In some embodiments, upon translation, the signal peptide is N-terminal to one or more neoepitope sequence(s) encoded by the RNA vaccine. In some embodiments, the secretory signal peptide comprises the sequence MRVMAPRTLILLLSGALALTETWAGS (SEQ ID NO:27). In some embodiments, the secretory signal peptide of the RNA vaccine comprises the sequence AUGAGAGUGAUGGCCCCCAGAACCCUGAUCCUGCUGCUGUCUGGCGCCCUGGCCCUGACA GAGACAUGGGCCGGAAGC (SEQ ID NO:25). In some embodiments, the secretory signal peptide of the RNA vaccine is encoded by DNA comprising the sequence

(SEQ ID NO: 26)
ATGAGAGTGATGGCCCCCAGAACCCTGATCCTGCTGCTGTCTGGCGCCCT
GGCCCTGACAGAGACATGGGCCGGAAGC.

In some embodiments, the RNA vaccine comprises a polynucleotide sequence encoding at least a portion of a transmembrane and/or cytoplasmic domain. In some embodiments, the transmembrane and/or cytoplasmic domains are from the transmembrane/cytoplasmic domains of an MHC molecule. The term “major histocompatibility complex” and the abbreviation “MHC” relate to a complex of genes which occurs in all vertebrates. The function of MHC proteins or molecules in signaling between lymphocytes and antigen-presenting cells in normal immune responses involves them binding peptides and presenting them for possible recognition by T-cell receptors (TCR). MHC molecules bind peptides in an intracellular processing compartment and present these peptides on the surface of antigen-presenting cells to T cells. The human MHC region, also referred to as HLA, is located on chromosome 6 and comprises the class I region and the class II region. The class I alpha chains are glycoproteins having a molecular weight of about 44 kDa. The polypeptide chain has a length of somewhat more than 350 amino acid residues. It can be divided into three functional regions: an external, a transmembrane and a cytoplasmic region. The external region has a length of 283 amino acid residues and is divided into three domains, alpha1, alpha2 and alpha3. The domains and regions are usually encoded by separate exons of the class I gene. The transmembrane region spans the lipid bilayer of the plasma membrane. It consists of 23 usually hydrophobic amino acid residues which are arranged in an alpha helix. The cytoplasmic region, i.e. the part which faces the cytoplasm and which is connected to the transmembrane region, typically has a length of 32 amino acid residues and is able to interact with the elements of the cytoskeleton. The alpha chain interacts with beta2-microglobulin and thus forms alpha-beta2 dimers on the cell surface. The term “MHC class II” or “class II” relates to the major histocompatibility complex class II proteins or genes. Within the human MHC class II region there are the DP, DQ and DR subregions for class II alpha chain genes and beta chain genes (i.e. DPalpha, DPbeta, DQalpha, DQbeta, DRalpha and DRbeta). Class II molecules are heterodimers each consisting of an alpha chain and a beta chain. Both chains are glycoproteins having a molecular weight of 31-34 kDa (a) or 26-29 kDA (beta). The total length of the alpha chains varies from 229 to 233 amino acid residues, and that of the beta chains from 225 to 238 residues. Both alpha and beta chains consist of an external region, a connecting peptide, a transmembrane region and a cytoplasmic tail. The external region consists of two domains, alpha1 and alpha2 or beta1 and beta2. The connecting peptide is respectively beta and 9 residues long in alpha and beta chains. It connects the two domains to the transmembrane region which consists of 23 amino acid residues both in alpha chains and in beta chains. The length of the cytoplasmic region, i.e. the part which faces the cytoplasm and which is connected to the transmembrane region, varies from 3 to 16 residues in alpha chains and from 8 to 20 residues in beta chains. Exemplary transmembrane/cytoplasmic domain sequences are described in U.S. Pat. Nos. 8,178,653 and 8,637,006. In some embodiments, upon translation, the transmembrane and/or cytoplasmic domain is C-terminal to one or more neoepitope sequence(s) encoded by the RNA vaccine. In some embodiments, the transmembrane and/or cytoplasmic domain of the MHC molecule encoded by the RNA vaccine comprises the sequence IVGIVAGLAVLAVVVIGAVVATVMCRRKSSGGKGGSYSQAASSDSAQGSDVSLTA (SEQ ID NO:30). In some embodiments, the transmembrane and/or cytoplasmic domain of the MHC molecule comprises the sequence AUCGUGGGAAUUGUGGCAGGACUGGCAGUGCUGGCCGUGGUGGUGAUCGGAGCCGUGGU GGCUACCGUGAUGUGCAGACGGAAGUCCAGCGGAGGCAAGGGCGGCAGCUACAGCCAGGC CGCCAGCUCUGAUAGCGCCCAGGGCAGCGACGUGUCACUGACAGCC (SEQ ID NO:28). In some embodiments, the transmembrane and/or cytoplasmic domain of the MHC molecule is encoded by DNA comprising the sequence

(SEQ ID NO: 29)
ATCGTGGGAATTGTGGCAGGACTGGCAGTGCTGGCCGTGGTGGTGATCGG
AGCCGTGGTGGCTACCGTGATGTGCAGACGGAAGTCCAGCGGAGGCAAGG
GCGGCAGCTACAGCCAGGCCGCCAGCTCTGATAGCGCCCAGGGCAGCGAC
GTGTCACTGACAGCC.

In some embodiments, the RNA vaccine comprises both a polynucleotide sequence encoding a secretory signal peptide that is N-terminal to the one or more neoepitope sequence(s) and a polynucleotide sequence encoding a transmembrane and/or cytoplasmic domain that is C-terminal to the one or more neoepitope sequence(s). Combining such sequences has been shown to improve processing and presentation of MHC Class I and II epitopes in human dendritic cells. See, e.g., Kreiter, S. et al. (2008) J. Immunol. 180:309-318.

In myeloid DCs, the RNA is released into the cytosol and translated into a poly-neoepitopic peptide. The polypeptide contains additional sequences to enhance antigen presentation. In some embodiments, a signal sequence (sec) from the MHCI heavy chain at the N-terminal of the polypeptide is used to target the nascent molecule to the endoplasmic reticulum, which has been shown to enhance MHCI presentation efficiency. Without wishing to be bound by theory, it is believed that the transmembrane and cytoplasmic domains of MHCI heavy chain guide the polypeptide to the endosomal/lysosomal compartments that were shown to improve MHCII presentation.

In some embodiments, the RNA vaccine comprises a 3′UTR. Certain untranslated sequences found 3′ to protein-coding sequences in mRNAs have been shown to improve RNA stability, translation, and protein expression. Polynucleotide sequences suitable for use as 3′ UTRs are described, for example, in PG Pub. No. US20190071682. In some embodiments, the 3′ UTR comprises the 3′ untranslated region of AES or a fragment thereof and/or the non-coding RNA of the mitochondrially encoded 12S RNA. The term “AES” relates to Amino-Terminal Enhancer Of Split and includes the AES gene (see, e.g., NCBI Gene ID:166). The protein encoded by this gene belongs to the groucho/TLE family of proteins, can function as a homooligomer or as a heteroologimer with other family members to dominantly repress the expression of other family member genes. An exemplary AES mRNA sequence is provided in NCBI Ref. Seq. Accession NO. NM_198969. The term “MT_RNR1” relates to Mitochondrially Encoded 12S RNA and includes the MT_RNR1 gene (see, e.g., NCBI Gene ID:4549). This RNA gene belongs to the Mt_rRNA class. Diseases associated with MT-RNR1 include restrictive cardiomyopathy and auditory neuropathy. Among its related pathways are Ribosome biogenesis in eukaryotes and CFTR translational fidelity (class I mutations). An exemplary MT_RNR1 RNA sequence is present within the sequence of NCBI Ref. Seq. Accession NO. NC_012920. In some embodiments, the 3′ UTR of the RNA vaccine comprises the sequence

CUGGUACUGCAUGCACGCAAUGCUAGCUGCCCCUUUCCCGUCCUGGGUACCCCGAGUCUC CCCCGACCUCGGGUCCCAGGUAUGCUCCCACCUCCACCUGCCCCACUCACCACCUCUGCUA GUUCCAGACACCUCC (SEQ ID NO:33). In some embodiments, the 3′ UTR of the RNA vaccine comprises the sequence
CAAGCACGCAGCAAUGCAGCUCAAAACGCUUAGCCUAGCCACACCCCCACGGGAAACAGC AGUGAUUAACCUUUAGCAAUAAACGAAAGUUUAACUAAGCUAUACUAACCCCAGGGUUG GUCAAUUUCGUGCCAGCCACACCG (SEQ ID NO:35). In some embodiments, the 3′ UTR of the RNA vaccine comprises the sequence
CUGGUACUGCAUGCACGCAAUGCUAGCUGCCCCUUUCCCGUCCUGGGUACCCCGAGUCUC CCCCGACCUCGGGUCCCAGGUAUGCUCCCACCUCCACCUGCCCCACUCACCACCUCUGCUA GUUCCAGACACCUCC (SEQ ID NO:33) and the sequence CAAGCACGCAGCAAUGCAGCUCAAAACGCUUAGCCUAGCCACACCCCCACGGGAAACAGC AGUGAUUAACCUUUAGCAAUAAACGAAAGUUUAACUAAGCUAUACUAACCCCAGGGUUG GUCAAUUUCGUGCCAGCCACACCG (SEQ ID NO:35). In some embodiments, the 3′ UTR of the RNA vaccine comprises the sequence
CUCGAGCUGGUACUGCAUGCACGCAAUGCUAGCUGCCCCUUUCCCGUCCUGGGUACCCCG AGUCUCCCCCGACCUCGGGUCCCAGGUAUGCUCCCACCUCCACCUGCCCCACUCACCACCU CUGCUAGUUCCAGACACCUCCCAAGCACGCAGCAAUGCAGCUCAAAACGCUUAGCCUAGC CACACCCCCACGGGAAACAGCAGUGAUUAACCUUUAGCAAUAAACGAAAGUUUAACUAAG CUAUACUAACCCCAGGGUUGGUCAAUUUCGUGCCAGCCACACCGAGACCUGGUCCAGAGU CGCUAGCCGCGUCGCU (SEQ ID NO:31). In some embodiments, the 3′ UTR of the RNA vaccine is encoded by DNA comprising the sequence
CTGGTACTGCATGCACGCAATGCTAGCTGCCCCTTTCCCGTCCTGGGTACCCCGAGTCTCCCC CGACCTCGGGTCCCAGGTATGCTCCCACCTCCACCTGCCCCACTCACCACCTCTGCTAGTTCC AGACACCTCC (SEQ ID NO:34). In some embodiments, the 3′ UTR of the RNA vaccine is encoded by DNA comprising the sequence
CAAGCACGCAGCAATGCAGCTCAAAACGCTTAGCCTAGCCACACCCCCACGGGAAACAGCA GTGATTAACCTTTAGCAATAAACGAAAGTTTAACTAAGCTATACTAACCCCAGGGTTGGTCA ATTTCGTGCCAGCCACACCG (SEQ ID NO:36). In some embodiments, the 3′ UTR of the RNA vaccine is encoded by DNA comprising the sequence
CTGGTACTGCATGCACGCAATGCTAGCTGCCCCTTTCCCGTCCTGGGTACCCCGAGTCTCCCC CGACCTCGGGTCCCAGGTATGCTCCCACCTCCACCTGCCCCACTCACCACCTCTGCTAGTTCC AGACACCTCC (SEQ ID NO:34) and the sequence CAAGCACGCAGCAATGCAGCTCAAAACGCTTAGCCTAGCCACACCCCCACGGGAAACAGCA GTGATTAACCTTTAGCAATAAACGAAAGTTTAACTAAGCTATACTAACCCCAGGGTTGGTCA ATTTCGTGCCAGCCACACCG (SEQ ID NO:36). In some embodiments, the 3′ UTR of the RNA vaccine is encoded by DNA comprising the sequence

(SEQ ID NO: 32)
CTGGTACTGCATGCACGCAATGCTAGCTGCCCCTTTCCCGTCCTGGGTAC
CCCGAGTCTCCCCCGACCTCGGGTCCCAGGTATGCTCCCACCTCCACCTG
CCCCACTCACCACCTCTGCTAGTTCCAGACACCTCCCAAGCACGCAGCAA
TGCAGCTCAAAACGCTTAGCCTAGCCACACCCCCACGGGAAACAGCAGTG
ATTAACCTTTAGCAATAAACGAAAGTTTAACTAAGCTATACTAACCCCAG
GGTTGGTCAATTTCGTGCCAGCCACACCGAGACCTGGTCCAGAGTCGCTA
GCCGCGTCGCT.

In some embodiments, the RNA vaccine comprises a poly(A) tail at its 3′end. In some embodiments, the poly(A) tail comprises more than 50 or more than 100 adenine nucleotides. For example, in some embodiments, the poly(A) tail comprises 120 adenine nucleotides. This poly(A) tail has been demonstrated to enhance RNA stability and translation efficiency (Holtkamp, S. et al. (2006) Blood 108:4009-4017). In some embodiments, the RNA comprising a poly(A) tail is generated by transcribing a DNA molecule comprising in the 5′→3′ direction of transcription, a polynucleotide sequence that encodes at least 50, 100, or 120 adenine consecutive nucleotides and a recognition sequence for a type IIS restriction endonuclease. Exemplary poly(A) tail and 3′ UTR sequences that improve translation are found, e.g., in U.S. Pat. No. 9,476,055.

In some embodiments, an RNA vaccine or molecule of the present disclosure comprises the general structure (in the 5′→3′ direction): (1) a 5′ cap; (2) a 5′ untranslated region (UTR); (3) a polynucleotide sequence encoding a secretory signal peptide; (4) a polynucleotide sequence encoding at least a portion of a transmembrane and cytoplasmic domain of a major histocompatibility complex (MHC) molecule; (5) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (6) a poly(A) sequence. In some embodiments, an RNA vaccine or molecule of the present disclosure comprises, in the 5′→3′ direction: the polynucleotide sequence GGCGAACUAGUAUUCUUCUGGUCCCCACAGACUCAGAGAGAACCCGCCACCAUGAGAGUG AUGGCCCCCAGAACCCUGAUCCUGCUGCUGUCUGGCGCCCUGGCCCUGACAGAGACAUGG GCCGGAAGC (SEQ ID NO:19); and the polynucleotide sequence

AUCGUGGGAAUUGUGGCAGGACUGGCAGUGCUGGCCGUGGUGGUGAUCGGAGCCGUGGU GGCUACCGUGAUGUGCAGACGGAAGUCCAGCGGAGGCAAGGGCGGCAGCUACAGCCAGGC CGCCAGCUCUGAUAGCGCCCAGGGCAGCGACGUGUCACUGACAGCCUAGUAACUCGAGCU GGUACUGCAUGCACGCAAUGCUAGCUGCCCCUUUCCCGUCCUGGGUACCCCGAGUCUCCC CCGACCUCGGGUCCCAGGUAUGCUCCCACCUCCACCUGCCCCACUCACCACCUCUGCUAGU UCCAGACACCUCCCAAGCACGCAGCAAUGCAGCUCAAAACGCUUAGCCUAGCCACACCCC CACGGGAAACAGCAGUGAUUAACCUUUAGCAAUAAACGAAAGUUUAACUAAGCUAUACU AACCCCAGGGUUGGUCAAUUUCGUGCCAGCCACACCGAGACCUGGUCCAGAGUCGCUAGC CGCGUCGCU (SEQ ID NO:20). Advantageously, RNA vaccines comprising this combination and orientation of structures or sequences are characterized by one or more of: improved RNA stability, enhanced translational efficiency, improved antigen presentation and/or processing (e.g., by DCs), and increased protein expression.

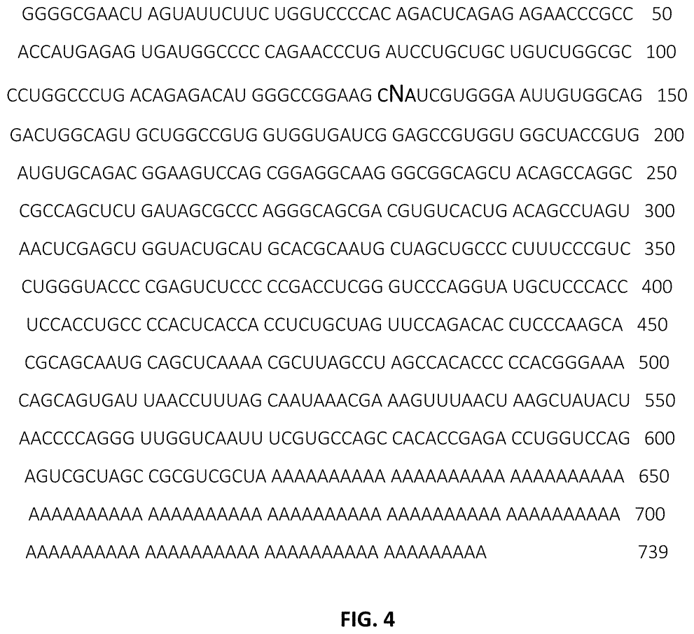

In some embodiments, an RNA vaccine or molecule of the present disclosure comprises the sequence (in the 5′→3′ direction) of SEQ ID NO:42. See, e.g., FIG. 4. In some embodiments, N refers to a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different neoepitopes. In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules). In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules) and an additional amino acid linker at the 3′ end.

In some embodiments, the RNA vaccine or molecule further comprises a polynucleotide sequence encoding at least one neoepitopes; wherein the polynucleotide sequence encoding the at least one neoepitope is between the polynucleotide sequence encoding the secretory signal peptide and the polynucleotide sequence encoding the at least portion of the transmembrane and cytoplasmic domain of the MHC molecule in the 5′→3′ direction. In some embodiments, the RNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes.

In some embodiments, the RNA vaccine or molecule further comprises, in the 5′→3′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′→3′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the MHC molecule, or between the sequences of SEQ ID NO:19 and SEQ ID NO:20, in the 5′→3′ direction. In some embodiments, the RNA vaccine or molecule comprises 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 linker-epitope modules. In some embodiments, each of the linker-epitope modules encodes a different neoepitope. In some embodiments, the RNA vaccine or molecule comprises 2, 3, 4, 5, 6, 7, 8, 9, 10, 11, 12, 13, 14, 15, 16, 17, 18, 19, or 20 linker-epitope modules, and the RNA vaccine or molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA vaccine or molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

In some embodiments, the RNA vaccine is at least 800 nucleotides, at least 1000 nucleotides, or at least 1200 nucleotides in length. In some embodiments, the RNA vaccine is less than 2000 nucleotides in length. In some embodiments, the RNA vaccine is at least 800 nucleotides but less than 2000 nucleotides in length, at least 1000 nucleotides but less than 2000 nucleotides in length, at least 1200 nucleotides but less than 2000 nucleotides in length, at least 1400 nucleotides but less than 2000 nucleotides in length, at least 800 nucleotides but less than 1400 nucleotides in length, or at least 800 nucleotides but less than 2000 nucleotides in length. For example, the constant regions of an RNA vaccine comprising the elements described above are approximately 800 nucleotides in length. In some embodiments, an RNA vaccine comprising 5 patient-specific neoepitopes (e.g., each encoding 27 amino acids) is greater than 1300 nucleotides in length. In some embodiments, an RNA vaccine comprising 10 patient-specific neoepitopes (e.g., each encoding 27 amino acids) is greater than 1800 nucleotides in length.

In some embodiments, the RNA vaccine is formulated in a lipoplex nanoparticle or liposome. In some embodiments, a lipoplex nanoparticle formulation for the RNA (RNA-Lipoplex) is used to enable IV delivery of an RNA vaccine of the present disclosure. In some embodiments, a lipoplex nanoparticle formulation for the RNA cancer vaccine comprising the synthetic cationic lipid (R)—N,N,N-trimethyl-2,3-dioleyloxy-1-propanaminium chloride (DOTMA) and the phospholipid 1,2-dioleoyl-sn-glycero-3-phosphoethanolamine (DOPE) is used, e.g., to enable IV delivery. The DOTMA/DOPE liposomal component has been optimized for IV delivery and targeting of antigen-presenting cells in the spleen and other lymphoid organs.

In one embodiment, the nanoparticles comprise at least one lipid. In one embodiment, the nanoparticles comprise at least one cationic lipid. The cationic lipid can be monocationic or polycationic. Any cationic amphiphilic molecule, eg, a molecule which comprises at least one hydrophilic and lipophilic moiety is a cationic lipid within the meaning of the present invention. In one embodiment, the positive charges are contributed by the at least one cationic lipid and the negative charges are contributed by the RNA. In one embodiment, the nanoparticles comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid.

In one embodiment, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof.

In one embodiment, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof.

In one embodiment, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In one embodiment, in this ratio, the molar amount of the cationic lipid results from the molar amount of the cationic lipid multiplied by the number of positive charges in the cationic lipid.

In one embodiment, the lipid is comprised in a vesicle encapsulating said RNA. The vesicle may be a multilamellar vesicle, an unilamellar vesicle, or a mixture thereof. The vesicle may be a liposome.

Nanoparticles or liposomes described herein can be formed by adjusting a positive to negative charge, depending on the (+/−) charge ratio of a cationic lipid to RNA and mixing the RNA and the cationic lipid. The +/− charge ratio of the cationic lipid to the RNA in the nanoparticles described herein can be calculated by the following equation. (+/− charge ratio)=[(cationic lipid amount (mol))*(the total number of positive charges in the cationic lipid)]:[(RNA amount (mol))*(the total number of negative charges in RNA)]. The RNA amount and the cationic lipid amount can be easily determined by one skilled in the art in view of a loading amount upon preparation of the nanoparticles. For further descriptions of exemplary nanoparticles, see, e.g., PG Pub. No. US20150086612.

In one embodiment, the overall charge ratio of positive charges to negative charges in the nanoparticles or liposomes (e.g., at physiological pH) is between 1.4:1 and 1:8, preferably between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In some embodiments, at physiological pH the overall charge ratio of positive charges to negative charges of the nanoparticles is between 1:1.2 (0.83) and 1:2 (0.5). In some embodiments, at physiological pH the overall charge ratio of positive charges to negative charges of the nanoparticles or liposomes is between 1.6:2 (0.8) and 1:2 (0.5) or between 1.6:2 (0.8) and 1.1:2 (0.55). In some embodiments, at physiological pH the overall charge ratio of positive charges to negative charges of the nanoparticles or liposomes is 1.3:2 (0.65). In some embodiments, at physiological pH the overall charge ratio of positive charges to negative charges of the liposome is not lower than 1.0:2.0. In some embodiments, at physiological pH the overall charge ratio of positive charges to negative charges of the liposome is not higher than 1.9:2.0. In some embodiments, at physiological pH the overall charge ratio of positive charges to negative charges of the liposome is not lower than 1.0:2.0 and not higher than 1.9:2.0.

In one embodiment, the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the nanoparticles are lipoplexes comprising DOTMA and DOPE in a molar ratio of 2:1 to 1:2, preferably 2:1 to 1:1, and wherein the charge ratio of positive charges in DOTMA to negative charges in the RNA is 1.4:1 or less. In one embodiment, the nanoparticles are lipoplexes comprising DOTMA and cholesterol in a molar ratio of 2:1 to 1:2, preferably 2:1 to 1:1, and wherein the charge ratio of positive charges in DOTMA to negative charges in the RNA is 1.4:1 or less. In one embodiment, the nanoparticles are lipoplexes comprising DOTAP and DOPE in a molar ratio of 2:1 to 1:2, preferably 2:1 to 1:1, and wherein the charge ratio of positive charges in DOTAP to negative charges in the RNA is 1.4:1 or less.

In one embodiment, the zeta potential of the nanoparticles or liposomes is −5 or less, −10 or less, −15 or less, −20 or less or −25 or less. In various embodiments, the zeta potential of the nanoparticles or liposomes is −35 or higher, −30 or higher or −25 or higher. In one embodiment, the nanoparticles or liposomes have a zeta potential from 0 mV to −50 mV, preferably 0 mV to −40 mV or −10 mV to −30 mV.

In some embodiments, the polydispersity index of the nanoparticles or liposomes is 0.5 or less, 0.4 or less, or 0.3 or less, as measured by dynamic light scattering.

In some embodiments, the nanoparticles or liposomes have an average diameter in the range of about 50 nm to about 1000 nm, from about 100 nm to about 800 nm, from about 200 nm to about 600 nm, from about 250 nm to about 700 nm, or from about 250 nm to about 550 nm, as measured by dynamic light scattering.

In some embodiments, the PCV is administered intravenously, for example, in a liposomal formulation, at doses of 15 μg, 25 μg, 38 μg, 50 μg, or 100 μg. In some embodiments, 15 μg, 25 μg, 38 μg, 50 μg, or 100 μg of RNA is delivered per dose (i.e., dose weight reflects the weight of RNA administered, not the total weight of the formulation or lipoplex administered). More than one PCV may be administered to a subject, e.g., subject is administered one PCV with a combination of neoepitopes and also administered a separate PCV with a different combination of neoepitopes. In some embodiments, a first PCV with ten neoepitopes is administered in combination with a second PCV with ten alternative epitopes.

In some embodiments, the PCV is administered such that it is delivered to the spleen. For example, the PCT can be administered such that one or more antigen(s) (e.g., patient-specific neo-antigens) are delivered to antigen presenting cells (e.g., in the spleen).

Any of the PCVs or RNA vaccines of the present disclosure may find use in the methods described herein. For example, in some embodiments, a PD-1 axis binding antagonist of the present disclosure is administered in combination with a personalized cancer vaccine (PCV), e.g., an RNA vaccine described supra.

Further provided herein are DNA molecules encoding any of the RNA vaccines of the present disclosure. For example, in some embodiments, a DNA molecule of the present disclosure comprises the general structure (in the 5′→3′ direction): (1) a polynucleotide sequence encoding a 5′ untranslated region (UTR); (2) a polynucleotide sequence encoding a secretory signal peptide; (3) a polynucleotide sequence encoding at least a portion of a transmembrane and cytoplasmic domain of a major histocompatibility complex (MHC) molecule; (4) a polynucleotide sequence encoding a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (5) a polynucleotide sequence encoding a poly(A) sequence. In some embodiments, a DNA molecule of the present disclosure comprises, in the 5′→3′ direction: the polynucleotide sequence GGCGAACTAGTATTCTTCTGGTCCCCACAGACTCAGAGAGAACCCGCCACCATGAGAGTGAT GGCCCCCAGAACCCTGATCCTGCTGCTGTCTGGCGCCCTGGCCCTGACAGAGACATGGGCCG GAAGC (SEQ ID NO:40); and the polynucleotide sequence

(SEQ ID NO: 41)
ATCGTGGGAATTGTGGCAGGACTGGCAGTGCTGGCCGTGGTGGTGATCGG
AGCCGTGGTGGCTACCGTGATGTGCAGACGGAAGTCCAGCGGAGGCAAGG
GCGGCAGCTACAGCCAGGCCGCCAGCTCTGATAGCGCCCAGGGCAGCGAC
GTGTCACTGACAGCCTAGTAACTCGAGCTGGTACTGCATGCACGCAATGC
TAGCTGCCCCTTTCCCGTCCTGGGTACCCCGAGTCTCCCCCGACCTCGGG
TCCCAGGTATGCTCCCACCTCCACCTGCCCCACTCACCACCTCTGCTAGT
TCCAGACACCTCCCAAGCACGCAGCAATGCAGCTCAAAACGCTTAGCCTA
GCCACACCCCCACGGGAAACAGCAGTGATTAACCTTTAGCAATAAACGAA
AGTTTAACTAAGCTATACTAACCCCAGGGTTGGTCAATTTCGTGCCAGCC
ACACCGAGACCTGGTCCAGAGTCGCTAGCCGCGTCGCT.

In some embodiments, the DNA molecule further comprises, in the 5′→3′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′→3′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the MHC molecule, or between the sequences of SEQ ID NO:40 and SEQ ID NO:41, in the 5′→3′ direction. In some embodiments, the DNA molecule comprises 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 linker-epitope modules, and each of the linker-epitope modules encodes a different neoepitope. In some embodiments, the DNA molecule comprises 2, 3, 4, 5, 6, 7, 8, 9, 10, 11, 12, 13, 14, 15, 16, 17, 18, 19, or 20 linker-epitope modules, and the DNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the DNA molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

Also provided herein are methods of producing any of the RNA vaccine of the present disclosure, comprising transcribing (e.g., by transcription of linear, double-stranded DNA or plasmid DNA, such as by in vitro transcription) a DNA molecule of the present disclosure. In some embodiments, the methods further comprise isolating and/or purifying the transcribed RNA molecule from the DNA molecule.

In some embodiments, an RNA or DNA molecule of the present disclosure comprises a type IIS restriction cleavage site, which allows RNA to be transcribed under the control of a 5′ RNA polymerase promoter and which contains a polyadenyl cassette (poly(A) sequence), wherein the recognition sequence is located 3′ of the poly(A) sequence, while the cleavage site is located upstream and thus within the poly(A) sequence. Restriction cleavage at the type IIS restriction cleavage site enables a plasmid to be linearized within the poly(A) sequence, as described in U.S. Pat. Nos. 9,476,055 and 10,106,800. The linearized plasmid can then be used as template for in vitro transcription, the resulting transcript ending in an unmasked poly(A) sequence. Any of the type IIS restriction cleavage sites described in U.S. Pat. Nos. 9,476,055 and 10,106,800 may be used.

### IV. PD-1 Axis Binding Antagonists

In some embodiments, a PCV (e.g., an RNA vaccine) of the present disclosure is administered in combination with a PD-1 axis binding antagonist.

For example, a PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partner(s). In a specific aspect the PD-1 ligand binding partners are PDL1 and/or PDL2. In another embodiment, a PDL1 binding antagonist is a molecule that inhibits the binding of PDL1 to its binding partner(s). In a specific aspect, PDL1 binding partner(s) are PD-1 and/or B7-1. In another embodiment, the PDL2 binding antagonist is a molecule that inhibits the binding of PDL2 to its binding partner(s). In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody).

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168. In some embodiments, the anti-PD-1 antibody comprises a heavy chain and a light chain sequence, wherein:
(a) the heavy chain comprises the amino acid sequence:

(SEQ ID NO: 11)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLG.

and
(b) the light chain comprises the amino acid sequence:

(SEQ ID NO: 12)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some embodiments, the anti-PD-1 antibody comprises the six HVR sequences from SEQ ID NO:11 and SEQ ID NO:12 (e.g., the three heavy chain HVRs from SEQ ID NO:11 and the three light chain HVRs from SEQ ID NO:12). In some embodiments, the anti-PD-1 antibody comprises the heavy chain variable domain from SEQ ID NO:11 and the light chain variable domain from SEQ ID NO:12.

In some embodiments, the anti-PD-1 antibody is pembrolizumab (CAS Registry Number: 1374853-91-4). Pembrolizumab (Merck), also known as MK-3475, Merck 3475, lambrolizumab, KEYTRUDA®, and SCH-900475, is an anti-PD-1 antibody described in WO2009/114335. In some embodiments, the anti-PD-1 antibody comprises a heavy chain and a light chain sequence, wherein:
(a) the heavy chain comprises the amino acid sequence:

(SEQ ID NO: 13)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG,

and
(b) the light chain comprises the amino acid sequence:

(SEQ ID NO: 14)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In some embodiments, the anti-PD-1 antibody comprises the six HVR sequences from SEQ ID NO:13 and SEQ ID NO:14 (e.g., the three heavy chain HVRs from SEQ ID NO:13 and the three light chain HVRs from SEQ ID NO:14). In some embodiments, the anti-PD-1 antibody comprises the heavy chain variable domain from SEQ ID NO:13 and the light chain variable domain from SEQ ID NO:14.

In some embodiments, the anti-PD-1 antibody is MEDI-0680 (AMP-514; AstraZeneca). MEDI-0680 is a humanized IgG4 anti-PD-1 antibody.

In some embodiments, the anti-PD-1 antibody is PDR001 (CAS Registry No. 1859072-53-9; Novartis). PDR001 is a humanized IgG4 anti-PD1 antibody that blocks the binding of PDL1 and PDL2 to PD-1.

In some embodiments, the anti-PD-1 antibody is REGN2810 (Regeneron). REGN2810 is a human anti-PD1 antibody also known as LIBTAYO® and cemiplimab-rwlc.

In some embodiments, the anti-PD-1 antibody is BGB-108 (BeiGene). In some embodiments, the anti-PD-1 antibody is BGB-A317 (BeiGene).

In some embodiments, the anti-PD-1 antibody is JS-001 (Shanghai Junshi). JS-001 is a humanized anti-PD1 antibody.

In some embodiments, the anti-PD-1 antibody is STI-A1110 (Sorrento). STI-A1110 is a human anti-PD1 antibody.

In some embodiments, the anti-PD-1 antibody is INCSHR-1210 (Incyte). INCSHR-1210 is a human IgG4 anti-PD1 antibody.

In some embodiments, the anti-PD-1 antibody is PF-06801591 (Pfizer).

In some embodiments, the anti-PD-1 antibody is TSR-042 (also known as ANB011; Tesaro/AnaptysBio).

In some embodiments, the anti-PD-1 antibody is AM0001 (ARMO Biosciences).

In some embodiments, the anti-PD-1 antibody is ENUM 244C8 (Enumeral Biomedical Holdings). ENUM 244C8 is an anti-PD1 antibody that inhibits PD-1 function without blocking binding of PDL1 to PD-1.

In some embodiments, the anti-PD-1 antibody is ENUM 388D4 (Enumeral Biomedical Holdings). ENUM 388D4 is an anti-PD1 antibody that competitively inhibits binding of PDL1 to PD-1.

In some embodiments, the PD-1 antibody comprises the six HVR sequences (e.g., the three heavy chain HVRs and the three light chain HVRs) and/or the heavy chain variable domain and light chain variable domain from a PD-1 antibody described in WO2015/112800 (Applicant: Regeneron), WO2015/112805 (Applicant: Regeneron), WO2015/112900 (Applicant: Novartis), US20150210769 (Assigned to Novartis), WO2016/089873 (Applicant: Celgene), WO2015/035606 (Applicant: Beigene), WO2015/085847 (Applicants: Shanghai Hengrui Pharmaceutical/Jiangsu Hengrui Medicine), WO2014/206107 (Applicants: Shanghai Junshi Biosciences/Junmeng Biosciences), WO2012/145493 (Applicant: Amplimmune), U.S. Pat. No. 9,205,148 (Assigned to MedImmune), WO2015/119930 (Applicants: Pfizer/Merck), WO2015/119923 (Applicants: Pfizer/Merck), WO2016/032927 (Applicants: Pfizer/Merck), WO2014/179664 (Applicant: AnaptysBio), WO2016/106160 (Applicant: Enumeral), and WO2014/194302 (Applicant: Sorrento).

In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. AMP-224 (CAS Registry No. 1422184-00-6; GlaxoSmithKline/MedImmune), also known as B7-DCIg, is a PDL2-Fc fusion soluble receptor described in WO2010/027827 and WO2011/066342.

In some embodiments, the PD-1 binding antagonist is a peptide or small molecule compound. In some embodiments, the PD-1 binding antagonist is AUNP-12 (PierreFabre/Aurigene). See, e.g., WO2012/168944, WO2015/036927, WO2015/044900, WO2015/033303, WO2013/144704, WO2013/132317, and WO2011/161699.

In some embodiments, the PDL1 binding antagonist is a small molecule that inhibits PD-1. In some embodiments, the PDL1 binding antagonist is a small molecule that inhibits PDL1. In some embodiments, the PDL1 binding antagonist is a small molecule that inhibits PDL1 and VISTA. In some embodiments, the PDL1 binding antagonist is CA-170 (also known as AUPM-170). In some embodiments, the PDL1 binding antagonist is a small molecule that inhibits PDL1 and TIM3. In some embodiments, the small molecule is a compound described in WO2015/033301 and WO2015/033299.

In some embodiments, the PD-1 axis binding antagonist is an anti-PDL1 antibody. A variety of anti-PDL1 antibodies are contemplated and described herein. In any of the embodiments herein, the isolated anti-PDL1 antibody can bind to a human PDL1, for example a human PDL1 as shown in UniProtKB/Swiss-Prot Accession No. Q9NZQ7.1, or a variant thereof. In some embodiments, the anti-PDL1 antibody is capable of inhibiting binding between PDL1 and PD-1 and/or between PDL1 and B7-1. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a humanized antibody. In some embodiments, the anti-PDL1 antibody is a human antibody. Examples of anti-PDL1 antibodies useful for the methods of this invention, and methods for making thereof are described in PCT patent application WO 2010/077634 A1 and U.S. Pat. No. 8,217,149, which are incorporated herein by reference.

In some embodiments, the anti-PDL1 antibody comprises a heavy chain variable region and a light chain variable region, wherein:
(a) the heavy chain variable region comprises an HVR-H1, HVR-H2, and HVR-H3 sequence of GFTFSDSWIH (SEQ ID NO:1), AWISPYGGSTYYADSVKG (SEQ ID NO:2) and RHWPGGFDY (SEQ ID NO:3), respectively, and
(b) the light chain variable region comprises an HVR-L1, HVR-L2, and HVR-L3 sequence of RASQDVSTAVA (SEQ ID NO:4), SASFLYS (SEQ ID NO:5) and QQYLYHPAT (SEQ ID NO:6), respectively.

In some embodiments, the anti-PDL1 antibody is MPDL3280A, also known as atezolizumab and TECENTRIQ® (CAS Registry Number: 1422185-06-5), with a WHO Drug Information (International Nonproprietary Names for Pharmaceutical Substances), Proposed INN: List 112, Vol. 28, No. 4, published Jan. 16, 2015 (see page 485) described therein. In some embodiments, the anti-PDL1 antibody comprises a heavy chain and a light chain sequence, wherein:
(a) the heavy chain variable region sequence comprises the amino acid sequence:

(SEQ ID NO: 7)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

and
(b) the light chain variable region sequence comprises the amino acid sequence:

(SEQ ID NO: 8)
DIQMTQSPSSLSASVGDRVTITCRASQDVSTAVAWYQQKPGKAPKWYSAS
FLYSGVPSRFSGSGSGTDFTLTISSLQPEDFATYYCQQYLYHPATFGQGT
KVEIKR.

In some embodiments, the anti-PDL1 antibody comprises a heavy chain and a light chain sequence, wherein:
(a) the heavy chain comprises the amino acid sequence:

(SEQ ID NO: 9)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,

and
(b) the light chain comprises the amino acid sequence:

(SEQ ID NO: 10)
DIQMTQSPSSLSASVGDRVTITCRASQDVSTAVAWYQQKPGKAPKWYSAS
FLYSGVPSRFSGSGSGTDFTLTISSLQPEDFATYYCQQYLYHPATFGQGT
KVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGEC.

In some embodiments, the anti-PDL1 antibody is avelumab (CAS Registry Number: 1537032-82-8). Avelumab, also known as MSB0010718C, is a human monoclonal IgG1 anti-PDL1 antibody (Merck KGaA, Pfizer). In some embodiments, the anti-PDL1 antibody comprises a heavy chain and a light chain sequence, wherein:
(a) the heavy chain comprises the amino acid sequence:

(SEQ ID NO: 15)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVITVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG,

and
(b) the light chain comprises the amino acid sequence:

(SEQ ID NO: 16)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVLGQPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS.

In some embodiments, the anti-PDL1 antibody comprises the six HVR sequences from SEQ ID NO:15 and SEQ ID NO:16 (e.g., the three heavy chain HVRs from SEQ ID NO:15 and the three light chain HVRs from SEQ ID NO:16). In some embodiments, the anti-PDL1 antibody comprises the heavy chain variable domain from SEQ ID NO:15 and the light chain variable domain from SEQ ID NO:16.

In some embodiments, the anti-PDL1 antibody is durvalumab (CAS Registry Number: 1428935-60-7). Durvalumab, also known as MEDI4736, is an Fc optimized human monoclonal IgG1 kappa anti-PDL1 antibody (MedImmune, AstraZeneca) described in WO2011/066389 and US2013/034559. In some embodiments, the anti-PDL1 antibody comprises a heavy chain and a light chain sequence, wherein:
(a) the heavy chain comprises the amino acid sequence:

(SEQ ID NO: 17)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSP
G,

and
(b) the light chain comprises the amino acid sequence:

(SEQ ID NO: 18)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In some embodiments, the anti-PDL1 antibody comprises the six HVR sequences from SEQ ID NO:17 and SEQ ID NO:18 (e.g., the three heavy chain HVRs from SEQ ID NO:17 and the three light chain HVRs from SEQ ID NO:18). In some embodiments, the anti-PDL1 antibody comprises the heavy chain variable domain from SEQ ID NO:17 and the light chain variable domain from SEQ ID NO:18.

In some embodiments, the anti-PDL1 antibody is MDX-1105 (Bristol Myers Squibb). MDX-1105, also known as BMS-936559, is an anti-PDL1 antibody described in WO2007/005874.

In some embodiments, the anti-PDL1 antibody is LY3300054 (Eli Lilly).

In some embodiments, the anti-PDL1 antibody is STI-A1014 (Sorrento). STI-A1014 is a human anti-PDL1 antibody.

In some embodiments, the anti-PDL1 antibody is KN035 (Suzhou Alphamab). KN035 is single-domain antibody (dAB) generated from a camel phage display library.

In some embodiments, the anti-PDL1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some embodiments, the anti-PDL1 antibody is CX-072 (CytomX Therapeutics).

In some embodiments, the PDL1 antibody comprises the six HVR sequences (e.g., the three heavy chain HVRs and the three light chain HVRs) and/or the heavy chain variable domain and light chain variable domain from a PDL1 antibody described in US20160108123 (Assigned to Novartis), WO2016/000619 (Applicant: Beigene), WO2012/145493 (Applicant: Amplimmune), U.S. Pat. No. 9,205,148 (Assigned to MedImmune), WO2013/181634 (Applicant: Sorrento), and WO2016/061142 (Applicant: Novartis).

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A.

In a still further specific aspect, the antibody has reduced or minimal effector function. In a still further specific aspect the minimal effector function results from an “effector-less Fc mutation” or aglycosylation mutation. In still a further embodiment, the effector-less Fc mutation is an N297A or D265A/N297A substitution in the constant region. In some embodiments, the isolated anti-PDL1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In a still further embodiment, the present disclosure provides for compositions comprising any of the above described anti-PDL1 antibodies in combination with at least one pharmaceutically-acceptable carrier.

In a still further embodiment, the present disclosure provides for a composition comprising an anti-PDL1, an anti-PD-1, or an anti-PDL2 antibody or antigen binding fragment thereof as provided herein and at least one pharmaceutically acceptable carrier. In some embodiments, the anti-PDL1, anti-PD-1, or anti-PDL2 antibody or antigen binding fragment thereof administered to the individual is a composition comprising one or more pharmaceutically acceptable carrier. Any of the pharmaceutically acceptable carriers described herein or known in the art may be used.

### V. Antibody Preparation

The antibody described herein is prepared using techniques available in the art for generating antibodies, exemplary methods of which are described in more detail in the following sections.

The antibody is directed against an antigen of interest (e.g., PD-1 or PD-L1, such as a human PD-1 or PD-L1). Preferably, the antigen is a biologically important polypeptide and administration of the antibody to a mammal suffering from a disorder can result in a therapeutic benefit in that mammal.

In certain embodiments, an antibody provided herein has a dissociation constant (Kd) of ≤1 μM, ≤150 nM, ≤100 nM, ≤50 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest. The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CMS chips at ˜10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 106 M−1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Chimeric, Humanized and Human Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibody Fragments

Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to solid tumors. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

Single-Domain Antibodies

In some embodiments, an antibody of the present disclosure is a single-domain antibody. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 3. More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 3
Conservative Substitutions.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present disclosure may be made in order to create antibody variants with certain improved properties.

In one embodiment, antibody variants are provided comprising an Fc region wherein a carbohydrate structure attached to the Fc region has reduced fucose or lacks fucose, which may improve ADCC function. Specifically, antibodies are contemplated herein that have reduced fucose relative to the amount of fucose on the same antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In certain embodiments, the antibody is one wherein less than about 50%, 40%, 30%, 20%, 10%, or 5% of the N-linked glycans thereon comprise fucose. For example, the amount of fucose in such an antibody may be from 1% to 80%, from 1% to 65%, from 5% to 65% or from 20% to 40%. In certain embodiments, the antibody is one wherein none of the N-linked glycans thereon comprise fucose, i.e., wherein the antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. I Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, the antibody variants comprising an Fc region described herein are capable of binding to an FcγRIII. In certain embodiments, the antibody variants comprising an Fc region described herein have ADCC activity in the presence of human effector cells or have increased ADCC activity in the presence of human effector cells compared to the otherwise same antibody comprising a human wild-type IgG1Fc region.

Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues). In an exemplary embodiment, the antibody comprising the following amino acid substitutions in its Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.)). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

### VI. Pharmaceutical Compositions and Formulations

Also provided herein are pharmaceutical compositions and formulations, e.g., for the treatment of cancer. In some embodiments, the pharmaceutical compositions and formulations further comprise a pharmaceutically acceptable carrier.

After preparation of the antibody of interest (e.g., techniques for producing antibodies which can be formulated as disclosed herein are elaborated herein and are known in the art), the pharmaceutical formulation comprising it is prepared. In certain embodiments, the antibody to be formulated has not been subjected to prior lyophilization and the formulation of interest herein is an aqueous formulation. In certain embodiments, the antibody is a full length antibody. In one embodiment, the antibody in the formulation is an antibody fragment, such as an F(ab′)₂, in which case problems that may not occur for the full length antibody (such as clipping of the antibody to Fab) may need to be addressed. The therapeutically effective amount of antibody present in the formulation is determined by taking into account the desired dose volumes and mode(s) of administration, for example. From about 25 mg/mL to about 150 mg/mL, or from about 30 mg/mL to about 140 mg/mL, or from about 35 mg/mL to about 130 mg/mL, or from about 40 mg/mL to about 120 mg/mL, or from about 50 mg/mL to about 130 mg/mL, or from about 50 mg/mL to about 125 mg/mL, or from about 50 mg/mL to about 120 mg/mL, or from about 50 mg/mL to about 110 mg/mL, or from about 50 mg/mL to about 100 mg/mL, or from about 50 mg/mL to about 90 mg/mL, or from about 50 mg/mL to about 80 mg/mL, or from about 54 mg/mL to about 66 mg/mL is an exemplary antibody concentration in the formulation. In some embodiments, an anti-PDL1 antibody described herein (such as atezolizumab) is administered at a dose of about 1200 mg. In some embodiments, an anti-PD1 antibody described herein (such as pembrolizumab) is administered at a dose of about 200 mg. In some embodiments, an anti-PD1 antibody described herein (such as nivolumab) is administered at a dose of about 240 mg (e.g., every 2 weeks) or 480 mg (e.g., every 4 weeks).

In some embodiments, an RNA vaccine described herein is administered at a dose of about 15 μg, about 25 μg, about 38 μg, about 50 μg, or about 100 μg.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The composition and formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. The formulations to be used for in vivo administration are generally sterile. Sterility may be readily accomplished, e.g., by filtration through sterile filtration membranes.

Pharmaceutical formulations of atezolizumab and pembrolizumab are commercially available. For example, atezolizumab is known under the trade name (as described elsewhere herein) TECENTRIQ®. Pembrolizumab is known under the trade name (as described elsewhere herein) KEYTRUDA®. In some embodiments, atezolizumab and the RNA vaccine, or pembrolizumab and the RNA vaccine, are provided in separate containers. In some embodiments, atezolizumab and pembrolizumab are used and/or prepared for administration to an individual as described in the prescribing information available with the commercially available product.

### VII. Methods of Treatment

Provided herein are methods for treating or delaying progression of cancer in an individual, comprising administering to the individual an effective amount of a PD-1 axis binding antagonist and an RNA vaccine. In some embodiments, the individual is human.

Any of the PD-1 axis binding antagonists and RNA vaccines of the present disclosure may find use in the methods of treatment described herein. In some embodiments, the RNA vaccine comprises one or more polynucleotides encoding 10-20 neoepitopes resulting from cancer-specific somatic mutations present in the tumor specimen. In some embodiments, the RNA vaccine comprises one or more polynucleotides encoding 5-20 neoepitopes resulting from cancer-specific somatic mutations present in the tumor specimen. In some embodiments, the RNA vaccine is formulated in a lipoplex nanoparticle or liposome. In some embodiments, a lipoplex nanoparticle formulation for the RNA (RNA-Lipoplex) is used to enable IV delivery of an RNA vaccine of the present disclosure. In some embodiments, the PCV is administered intravenously, for example, in a liposomal formulation, at doses of 15 μg, 25 μg, 38 μg, 50 μg, or 100 μg. In some embodiments, 15 μg, 25 μg, 38 μg, 50 μg, or 100 μg of RNA is delivered per dose (i.e., dose weight reflects the weight of RNA administered, not the total weight of the formulation or lipoplex administered). More than one PCV may be administered to a subject, e.g., subject is administered one PCV with a combination of neoepitopes and also administered a separate PCV with a different combination of neoepitopes. In some embodiments, a first PCV with ten neoepitopes is administered in combination with a second PCV with ten alternative epitopes. In some embodiments, the PD-1 axis binding antagonist is an anti-PD-1 antibody, including without limitation pembrolizumab. In some embodiments, the PD-1 axis binding antagonist is an anti-PD-L1 antibody, including without limitation atezolizumab.

In some embodiments, the PD-1 axis binding antagonist is administered to the individual at an interval of 21 days or 3 weeks. In some embodiments, the PD-1 axis binding antagonist is an anti-PD-1 antibody (e.g., pembrolizumab) administered to the individual at an interval of 21 days or 3 weeks, e.g., at a dose of about 200 mg. In some embodiments, the PD-1 axis binding antagonist is an anti-PD-1 antibody (e.g., cemiplimab-rwlc) administered to the individual at an interval of 21 days or 3 weeks, e.g., at a dose of about 350 mg. In some embodiments, the PD-1 axis binding antagonist is an anti-PD-L1 antibody (e.g., atezolizumab) administered to the individual at an interval of 21 days or 3 weeks, e.g., at a dose of about 1200 mg.

In some embodiments, the PD-1 axis binding antagonist is administered to the individual at an interval of 14 days or 28 days. In some embodiments, the PD-1 axis binding antagonist is administered to the individual at an interval of 2 weeks or 4 weeks. In some embodiments, the PD-1 axis binding antagonist is an anti-PD-1 antibody (e.g., nivolumab) administered to the individual at an interval of 14 days, 2 weeks, 28 days, or 4 weeks, e.g., at a dose of about 240 mg at an interval of 14 days or 2 weeks, or at a dose of about 480 mg at an interval of 28 days or 4 weeks. In some embodiments, the PD-1 axis binding antagonist is an anti-PD-1 antibody (e.g., nivolumab) administered to the individual at an interval of 21 days or 3 weeks, e.g., at a dose of about 1 mg/kg for 1, 2, 3, or 4 doses, optionally in combination with an anti-CTLA-4 antibody (e.g., ipilimumab), and optionally followed by administration of the anti-PD-1 antibody (e.g., nivolumab) alone at an interval of 14 days, 2 weeks, 28 days, or 4 weeks, e.g., at a dose of about 240 mg at an interval of 14 days or 2 weeks, or at a dose of about 480 mg at an interval of 28 days or 4 weeks.

In some embodiments, the PD-1 axis binding antagonist is administered to the individual at an interval of 14 days or 2 weeks. In some embodiments, the PD-1 axis binding antagonist is an anti-PD-L1 antibody (e.g., durvalumab) administered to the individual at an interval of 14 days or 2 weeks, e.g., at a dose of about 10 mg/kg (optionally by intravenous infusion over 60 minutes). In some embodiments, the PD-1 axis binding antagonist is an anti-PD-L1 antibody (e.g., avelumab) administered to the individual at an interval of 14 days or 2 weeks, e.g., at a dose of about 10 mg/kg (optionally by intravenous infusion over 60 minutes).

In some embodiments, the RNA vaccine is administered to the individual at an interval of 21 days or 3 weeks.

In some embodiments, the PD-1 axis binding antagonist and the RNA vaccine are administered to the individual in 8 21-day Cycles. In some embodiments, the RNA vaccine is administered to the individual on Days 1, 8, and 15 of Cycle 2 and Day 1 of Cycles 3-7. In some embodiments, the PD-1 axis binding antagonist is administered to the individual on Day 1 of Cycles 1-8. In some embodiments, the RNA vaccine is administered to the individual on Days 1, 8, and 15 of Cycle 2 and Day 1 of Cycles 3-7, and the PD-1 axis binding antagonist is administered to the individual on Day 1 of Cycles 1-8.

In some embodiments, the PD-1 axis binding antagonist and the RNA vaccine are further administered to the individual after Cycle 8. In some embodiments, the PD-1 axis binding antagonist and the RNA vaccine are further administered to the individual in 17 additional 21-day Cycles, wherein the PD-1 axis binding antagonist is administered to the individual on Day 1 of Cycles 13-29, and/or wherein the RNA vaccine is administered to the individual on Day 1 of Cycles 13, 21, and 29.

In certain embodiments, a PD-1 axis binding antagonist and an RNA vaccine are administered to the individual in 8 21-day Cycles, wherein the PD-1 axis binding antagonist is pembrolizumab and is administered to the individual at a dose of about 200 mg on Day 1 of Cycles 1-8, and wherein the RNA vaccine is administered to the individual at a dose of about 25 μg on Days 1, 8, and 15 of Cycle 2 and Day 1 of Cycles 3-7. In certain embodiments, a PD-L1 axis binding antagonist and the RNA vaccine are administered to the individual in 8 21-day Cycles, wherein the PD-L1 axis binding antagonist is atezolizumab and is administered to the individual at a dose of about 1200 mg on Day 1 of Cycles 1-8, and wherein the RNA vaccine is administered to the individual at a dose of about 25 μg on Days 1, 8, and 15 of Cycle 2 and Day 1 of Cycles 3-7. In some embodiments, the RNA vaccine is administered to the individual at doses of about 25 μg on Day 1 of Cycle 2, about 25 μg on Day 8 of Cycle 2, about 25 μg on Day 15 of Cycle 2, and about 25 μg on Day 1 of each of Cycles 3-7 (that is to say, a total of about 75 μg of the vaccine is administered to the individual over 3 doses during Cycle 2). In some embodiments, a total of about 75 μg of the vaccine is administered to the individual over 3 doses during the first Cycle in which the RNA vaccine is administered.

In certain embodiments, a PD-1 axis binding antagonist and an RNA vaccine are administered to the individual in 8 21-day Cycles, wherein the PD-1 axis binding antagonist is pembrolizumab and is administered to the individual at a dose of 200 mg on Day 1 of Cycles 1-8, and wherein the RNA vaccine is administered to the individual at a dose of 25 μg on Days 1, 8, and 15 of Cycle 2 and Day 1 of Cycles 3-7. In certain embodiments, a PD-L1 axis binding antagonist and the RNA vaccine are administered to the individual in 8 21-day Cycles, wherein the PD-L1 axis binding antagonist is atezolizumab and is administered to the individual at a dose of 1200 mg on Day 1 of Cycles 1-8, and wherein the RNA vaccine is administered to the individual at a dose of 25 μg on Days 1, 8, and 15 of Cycle 2 and Day 1 of Cycles 3-7. In some embodiments, the RNA vaccine is administered to the individual at doses of 25 μg on Day 1 of Cycle 2, 25 μg on Day 8 of Cycle 2, 25 μg on Day 15 of Cycle 2, and 25 μg on Day 1 of each of Cycles 3-7 (that is to say, a total of 75 μg of the vaccine is administered to the individual over 3 doses during Cycle 2). In some embodiments, a total of 75 μg of the vaccine is administered to the individual over 3 doses during the first Cycle in which the RNA vaccine is administered.

The PD-1 axis binding antagonist and the RNA vaccine may be administered in any order. For example, a PD-1 axis binding antagonist and an RNA vaccine may be administered sequentially (at different times) or concurrently (at the same time). In some embodiments, a PD-1 axis binding antagonist and an RNA vaccine are in separate compositions. In some embodiments, a PD-1 axis binding antagonist and an RNA vaccine are in the same composition.

In some embodiments, the cancer is selected from the group consisting of melanoma, non-small cell lung cancer, bladder cancer, colorectal cancer, triple negative breast cancer, renal cancer, and head and neck cancer. In some embodiments, the cancer is locally advanced or metastatic melanoma, non-small cell lung cancer, bladder cancer, colorectal cancer, triple negative breast cancer, renal cancer, or head and neck cancer. In some embodiments, the cancer is selected from the group consisting of non-small cell lung cancer, bladder cancer, colorectal cancer, triple negative breast cancer, renal cancer, and head and neck cancer. In some embodiments, the cancer is locally advanced or metastatic non-small cell lung cancer, bladder cancer, colorectal cancer, triple negative breast cancer, renal cancer, or head and neck cancer.

In some embodiments, the cancer is melanoma. In some embodiments, the melanoma is cutaneous or mucosal melanoma. In some embodiments, the melanoma is cutaneous, mucosal, or acral melanoma. In some embodiments, the melanoma is not ocular or acral melanoma. In some embodiments, the melanoma is metastatic or unresectable locally advanced melanoma. In some embodiments, the melanoma is stage IV melanoma. In some embodiments, the melanoma is stage IIIC or stage IIID melanoma. In some embodiments, the melanoma is unresectable or metastatic melanoma. In some embodiments, the method provides adjuvant treatment of melanoma.

In some embodiments, the cancer (e.g., melanoma) is previously untreated. In some embodiments, the cancer is previously untreated advanced melanoma.

In some embodiments, prior to treatment with a PD-1 axis binding antagonist and an RNA vaccine according to any of the methods described herein, the individual has progressed after treatment with or failed to respond adequately to treatment with a PD-1 axis binding antagonist-based monotherapy, e.g., treatment with pembrolizumab in the absence of an RNA vaccine.

The PD-1 axis binding antagonist and the RNA vaccine may be administered by the same route of administration or by different routes of administration. In some embodiments, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some embodiments, the RNA vaccine is administered (e.g., in a lipoplex particle or liposome) intravenously, intramuscularly, subcutaneously, topically, orally, transdermally, intraperitoneally, intraorbitally, by implantation, by inhalation, intrathecally, intraventricularly, or intranasally. In some embodiments, the PD-1 axis binding antagonist and the RNA vaccine are administered via intravenous infusion. An effective amount of the PD-1 axis binding antagonist and the RNA vaccine may be administered for prevention or treatment of disease.

In some embodiments, the methods may further comprise an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation.

### VIII. Articles of Manufacture or Kits

Further provided herein is an article of manufacture or a kit comprising a PD-1 axis binding antagonist (such as atezolizumab or pembrolizumab). In some embodiments, the article of manufacture or kit further comprises package insert comprising instructions for using the PD-1 axis binding antagonist in conjunction with the RNA vaccine to treat or delay progression of cancer in an individual or to enhance immune function of an individual having cancer. Also provided herein is an article of manufacture or a kit comprising a PD-1 axis binding antagonist (such as atezolizumab or pembrolizumab) and an RNA vaccine.

In some embodiments, the PD-1 axis binding antagonist and the RNA vaccine are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: A Phase II, Open-Label, Multicenter, Randomized Study of the Efficacy and Safety of an RNA Vaccine in Combination with Pembrolizumab in Patients with Previously Untreated Advanced Melanoma

### Rationale

As noted above, checkpoint inhibitors are currently the standard of care for metastatic melanoma. However, the durable clinical benefit observed with agents targeting PD-L1/PD-1 across diverse malignancies, including melanoma, appears limited to a subset of patients. Despite the advances in OS that have accompanied the development of now widely administered immunotherapies such as PD-1 therapies (nivolumab, pembrolizumab), or the combination of anti-PD1 with anti-CTLA-4 therapy (nivolumab and ipilimumab), a significant fraction of patients do not respond to treatment with checkpoint inhibitors or experience only transient disease stabilization (Robert C, Long G V, Brady B, et al. N Engl J Med 2015a; 372:320-30; Rosenberg J E, Hoffman-Censits J, Powles T, et al. Lancet 2016; 387:1909-20), which demonstrates the persistent unmet need for patients with metastatic solid tumors. Although objective responses in the approximately 10%-30% of patients who respond to treatment with PD-1 inhibitors tend to be durable, these patients nonetheless remain at risk for progression. In a recent study of melanoma patients treated with PD-1 blockade, 53 out of 205 patients (26%) who had had an objective response to pembrolizumab had disease progression at a median follow-up of 21 months (Ribas A, Hamid O, Daud A, et al. JAMA 2016; 315:1600-9).

While anti-PD1 and anti-PD1 plus anti-CTLA-4 combinations have significantly improved long-term outcomes in patients with melanoma the latter has come at the cost of increased treatment related toxicities. Despite these improvements, a significant proportion of patients remains at risk of disease progression and succumb to their disease. Combination therapies that address mechanisms of resistance checkpoint blockade with increasing toxicity are needed.

Resistance may occur at the level of the effector T cell, whose activity may be limited due to poor T-cell stimulation. In preclinical models, induction of antigen specific immunity combined with concomitant blockade of PD-L1/PD-1 pathways demonstrated superior efficacy over the respective single-agent inhibitors of these pathways, even in models in which single-agent vaccine had limited activity. In these studies, tumor-infiltrating T cells demonstrated increased IFN-γ expression (a hallmark of activation and anti-tumor activity of T cells) only when PD-L1 was blocked but not with single-agent vaccine (Duraiswamy J, Kaluza K M, Freeman G J, et al. Cancer Res 2013; 73:3591-603; Fu J, Malm I J, Kadayakkara D K, et al. Cancer Res 2014; 74:4042-52). On the basis of these studies, it is hypothesized that the combination of RO7198457 with anti-PD-L1/PD-1 may result in activation of anti-tumor immune responses leading to enhanced killing of tumor cells and improved clinical responses in cancer patients.

### Objectives

This study evaluates the efficacy, safety, pharmacokinetics, and patient-reported outcomes (PROs) of a personalized RNA neo-epitope vaccine (PCV), RO7198457, plus pembrolizumab compared with pembrolizumab alone in patients with previously untreated advanced melanoma. Specific objectives and corresponding endpoints for the study are outlined below.

The primary efficacy objective for this study is to evaluate the efficacy of RO7198457 plus pembrolizumab compared with pembrolizumab alone on the basis of the following endpoints:
Progression-free survival (PFS) after randomization, defined as the time from randomization to the first occurrence of disease progression or death from any cause (whichever occurs first), as determined by the investigator according to Response Evaluation Criteria in Solid Tumors, Version 1.1 (RECIST v1.1)
Objective response rate (ORR), defined as the proportion of patients with a complete response

(CR) or partial response (PR) on two consecutive occasions 4 weeks apart, as determined by the investigator according to RECIST v1.1

A secondary efficacy objective for this study is to evaluate the efficacy of the RNA neo-epitope vaccine plus pembrolizumab compared with pembrolizumab alone on the basis of the following endpoints:
Overall survival (OS) after randomization, defined as the time from randomization to death from any cause
Duration of response (DOR), defined as the time from the first occurrence of a documented objective response to disease progression or death from any cause, as determined by the investigator according to RECIST v1.1
Mean change from baseline in health-related quality of life (HRQoL) scores as assessed through use of the two-item global health status (GHS)/HRQoL subscale (Questions 29 and 30) of the European Organisation for Research and Treatment of Cancer Quality of Life-Core 30 (EORTC QLQ-C30) at specified timepoints

Another secondary efficacy objective for this study is to evaluate the percentage of participants with an objective response of CR or PR following cross-over from pembrolizumab monotherapy to combination therapy (e.g., RNA neo-epitope vaccine plus pembrolizumab).

Another secondary objective is to evaluate the efficacy of the RNA neo-epitope vaccine plus pembrolizumab in patients who have progressed following pembrolizumab monotherapy on the basis of the following endpoint:
ORR, defined as the proportion of patients with a CR or PR on two consecutive occasions ≥4 weeks apart, as determined by the investigator according to RECIST v1.1, at the time of crossover

Another objective for this study is to evaluate the incidence and severity of Adverse Events (AEs).

### Study Design

This is a Phase II, randomized, open-label, multicenter study designed to evaluate the efficacy and safety of RO7198457 (PCV) plus pembrolizumab compared with pembrolizumab alone in patients with previously untreated advanced melanoma. The patient population includes patients with unresectable locally advanced (Stages IIIC and IIID) and metastatic (recurrent or de novo Stage IV) melanoma. This study is to be conducted globally.

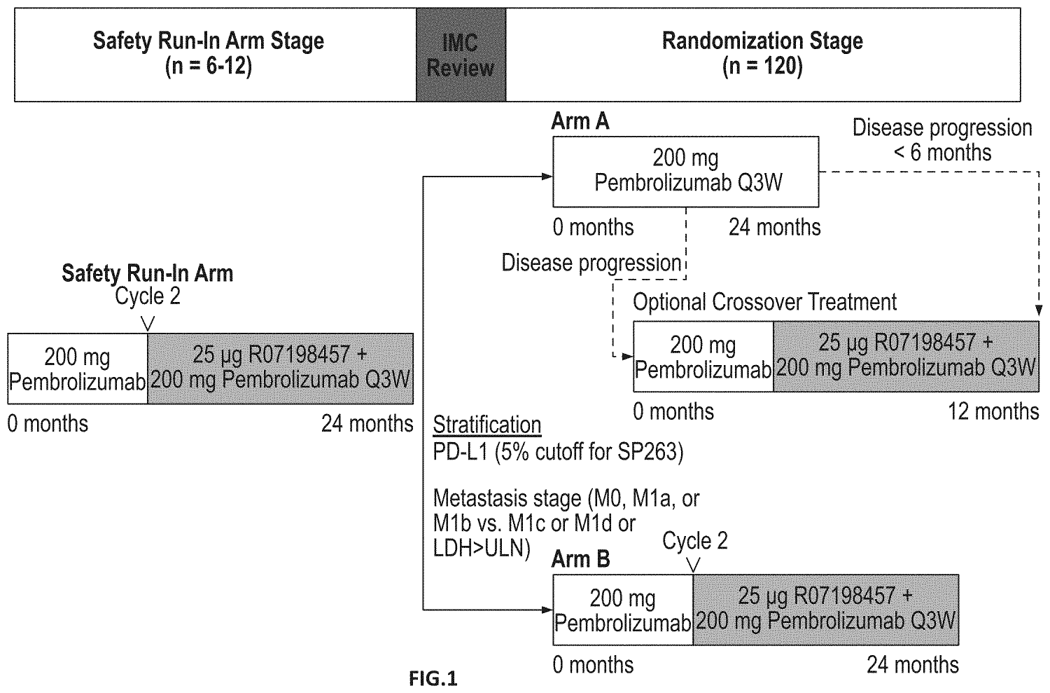

The study consists of two stages: an initial safety run-in stage and a randomized stage (FIG. 1). Each stage has a two-part screening period, a treatment period, and post-treatment follow-up period.

The safety run-in stage consists of a single arm that enrolls approximately 6-12 patients who receive 1 cycle (21 days) of 200 mg pembrolizumab administered by IV infusion followed by 25 μg RO7198457 plus 200 mg pembrolizumab IV every 3 weeks (Q3W) for subsequent cycles. Accrual in the randomized stage does not start until an Internal Monitoring Committee (IMC) has reviewed the safety data of the first 6 patients treated in the safety run-in stage.

The randomized stage enrolls approximately 120 patients, randomized in a 2:1 ratio, to either the experimental or control arm:
Arm A (control): 200 mg pembrolizumab administered by IV infusion Q3W
Arm B (experimental): 1 cycle of 200 mg pembrolizumab administered by IV infusion followed by 25 μg RO7198457 plus 200 mg pembrolizumab IV Q3W for subsequent cycles

Upon confirmed disease progression (as assessed by the investigator per RECIST v1.1), patients randomized to Arm A are given the option to cross over and receive combination treatment with RO7198457 and pembrolizumab, provided they meet eligibility criteria.

During the first part of the screening period (Part A), consenting patients are assessed for preliminary eligibility (e.g., Eastern Cooperative Oncology Group [ECOG] Performance Status, blood chemistry, serology for HIV, hepatitis B virus [HBV], and hepatitis C virus [HCV]) and tumor tissue and blood samples are collected to define tumor-specific somatic mutations and perform human leukocyte antigen (HLA)-typing to enable RO7198457 manufacturing. The current planned manufacturing turn-around time is approximately 4-6 weeks from receipt of blood samples and tumor samples of adequate quantity and quality. The second part of the screening period (Part B) is a 28-day period prior to Day 1 to confirm patient eligibility.

Eligible patients include male and female patients aged 18 years with ECOG Performance Status of 0 or 1 who have histologically confirmed Stage IIIC or IIID (unresectable) or metastatic (recurrent or de novo Stage IV) invasive cutaneous or mucosal melanoma that is measurable and who have not received prior treatment for advanced disease. Patients with ocular or acral melanoma or untreated CNS metastases are not eligible. Prior adjuvant therapy with ipilimumab, BRAF inhibitors, and/or MEK inhibitors is permitted. Prior adjuvant therapy with anti-PD-1/PD-L1 agents is permitted, provided the last dose was administered at least 6 months prior to Cycle 1, Day 1. Patients must be able to provide tumor specimens for vaccine manufacturing and PD-L1 testing.

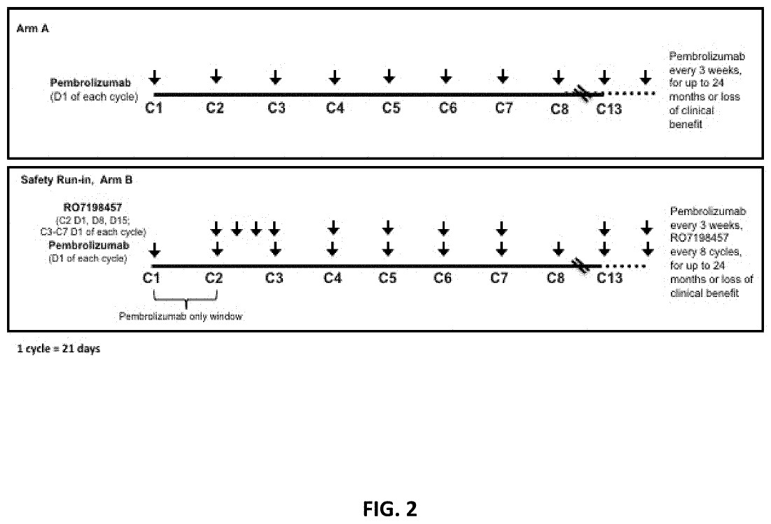

As shown in FIG. 2, patients in Arm A (pembrolizumab) receive 200 mg of pembrolizumab administered by IV infusion Q3W starting in Cycle 1. Patients in the safety run-in stage and Arm B of the randomized stage (25 μg RO7198457 plus 200 mg pembrolizumab) receive pembrolizumab administered by IV infusion Q3W starting in Cycle 1. Cycle 1 is a pembrolizumab monotherapy run-in to allow time for vaccine manufacturing. RO7198457 plus pembrolizumab start at Cycle 2, with RO7198457 administered by IV infusion 30 minutes after the completion of the pembrolizumab infusion. For the safety run-in stage and Arm B, RO7198457 dosing begins on Day 1 of Cycle 2 and is then administered on Days 8 and 15 of Cycle 2; Day 1 of Cycles 3-7 inclusive, and then as maintenance treatment every 8 cycles starting on Cycle 13 (Cycles 13, 21, and 29). Patients who experience a delay to the start of combination treatment with RO7198457 (e.g., RO7198457 not available by Day 1 of Cycle 2) or interruption during the RO7198457 induction may be permitted to start combination treatment later than Day 1 of Cycle 2 and/or to receive makeup doses of RO7198457 later in the initial treatment period to achieve a total of 8 induction doses, with Medical Monitor approval (e.g., patients who miss Day 1 of Cycle 2 would start RO7198457 on Day 8 of Cycle 2 and receive a makeup dose on Day 8 of Cycle 3 as an unscheduled visit, patients who start RO7198457 on Day 15 of Cycle 2 would receive makeup doses on both Days 8 and 15 of Cycle 3 as unscheduled visits, etc.).

The duration of treatment on this study is up to 24 months for all patients as long as they are experiencing clinical benefit as assessed by the investigator in the absence of unacceptable toxicity or symptomatic deterioration attributed to disease progression after an integrated assessment of radiographic data and clinical status. Patients may be permitted to continue treatment after RECIST v1.1 criteria for progressive disease are met. Patients in Arm A may have the option to cross over to combination treatment with RO7198457 plus pembrolizumab after confirmed disease progression, if crossover eligibility criteria are met. In addition, if a patient in Arm A completes 24 months of pembrolizumab and experiences confirmed disease progression <6 months after discontinuing pembrolizumab, they may have the option to receive crossover treatment with RO7198457 plus pembrolizumab.

Patients undergo tumor assessments at baseline (Cycle 1, Day 1), Week 12, and every 6 weeks (every 2 cycles) thereafter for the first 48 weeks following Cycle 1, Day 1. Digital photography of cutaneous lesions, if indicated, is performed at screening and at the first clinic visit following each tumor assessment. After 48 weeks from Cycle 1, Day 1, patients undergo tumor assessment every 12 (±1) weeks (approximately every 4 cycles). Tumor assessments continue until discontinuation of study treatment, withdrawal of consent, study termination by the Sponsor, or death, whichever occurs first. After experiencing disease progression that results in treatment discontinuation, patients are also asked to return to the clinic approximately 6 (±2) weeks later for confirmatory tumor assessments, if feasible. Patients who discontinue treatment for reasons other than disease progression (e.g., toxicity) should continue scheduled tumor assessments until disease progression, withdrawal of consent, study termination by Sponsor, or death, whichever occurs first. Primary imaging data used for tumor assessment is collected by the Sponsor to enable centralized, independent review of response endpoints if needed.

In addition, patients are also asked to complete PRO assessments at the beginning of each cycle until disease progression or treatment discontinuation, whichever occurs later.

### Inclusion and Exclusion Criteria

Patients must meet the following criteria for study entry:
Age ≥18 years at time of signing the Informed Consent Form
Histologically confirmed metastatic (recurrent or de novo Stage IV) or unresectable locally advanced (Stage IIIC or IIID) cutaneous or mucosal melanoma, as defined by the AJCC v8.0 (Amin M B, Edge S B, Greene F L, et al., editors. AJCC cancer staging manual. 8th rev ed. New York: Springer; 2017)
   The enrollment of mucosal melanoma patients is limited to approximately 10 patients
ECOG Performance Status of 0 or 1
Life expectancy ≥12 weeks
Adequate hematologic and end-organ function, defined by the following laboratory results obtained within 28 days prior to the first study treatment (Cycle 1, Day 1):
   ANC ≥1,500 cells/μL (without granulocyte colony-stimulating factor [G-CSF] support within 2 weeks prior to Cycle 1, Day 1)
   WBC count ≥2,500/μL
   Platelet count ≥100,000/μL (without transfusion within 14 days prior to Cycle 1, Day 1)
   Hemoglobin ≥9 g/dL (Patients may be transfused or may receive erythropoietic treatment as per local standard of care)
   Total bilirubin ≤1.5×ULN with the following exception: Patients with known Gilbert disease: serum bilirubin level ≤3×ULN.
   AST and ALT≤3×ULN
   ALP ≤2.5×ULN with the following exception: Patients with documented liver or bone metastases may have ALP ≤5×ULN.
   Serum albumin ≥2.5 g/dL
Measured or calculated creatinine CL ≥50 mL/min on the basis of the Cockcroft-Gault glomerular filtration rate estimation:

$$\frac{( 140 - age ) \times ( weight in kilograms ) \times ( 0.85 if female )}{72 \times ( serum creatinine in mg \text{/} dL )}$$

Measurable disease per RECIST v1.1. Previously irradiated lesions should not be counted as target lesions unless there has been demonstrated progression in the lesion and no other target lesions are available. Lesions that are intended to be biopsied should not be counted as target lesions. Cutaneous lesions and other superficial lesions that are detectable only by physical examination should not be counted as target lesions but may be included as non-target lesions.
Naive to prior systemic anti-cancer therapy for advanced melanoma (e.g., chemotherapy, hormonal therapy, targeted therapy, immunotherapy, or other biologic therapies), with the following exceptions for adjuvant therapies:
   Adjuvant treatment with anti-PD1/PD-L1 or anti-CTLA-4, if discontinued at least 6 months prior to Cycle 1, Day 1 and not meeting any of the following criteria:
      Any history of an immune-related Grade 4 adverse event attributed to prior CIT (other than endocrinopathy managed with replacement therapy or asymptomatic elevation of serum amylase or lipase)
      Any history of an immune-related Grade 3 adverse event attributed to prior CIT that required permanent discontinuation of the prior immunotherapeutic agent per local prescribing information, European Society for Medical Oncology (ESMO) guidelines (Haanen JBAG, Carbonnel F, Robert C, et al. Ann Oncol 2017; 28:iv119-iv142), or American Society of Clinical Oncology (ASCO) guidelines (Brahmer J R, Lacchetti C, Schneider B J, et al. J Clin Oncol 2018; 36:1714-68)
      Adverse events from prior anti-cancer therapy that have not resolved to Grade ≤1 except for alopecia, vitiligo, or endocrinopathy managed with replacement therapy. Patients with asymptomatic elevations of lipase/amylase may be eligible following discussion with the Medical Monitor.
      Immune-related adverse events related to prior CIT (other than endocrinopathy managed with replacement therapy or stable vitiligo) that have not resolved to baseline. Patients treated with corticosteroids for immune-related adverse events must demonstrate absence of related symptoms or signs for 4 weeks following discontinuation of corticosteroids.
   Adjuvant treatment with targeted therapies (e.g., BRAFi/MEKi), if discontinued at least 2 months prior to initiation of study treatment
   Adjuvant treatment with herbal therapies, if discontinued at least 7 days prior to initiation of study treatment
Confirmed availability of representative tumor specimens in formalin-fixed, paraffin-embedded blocks (preferred), or sectioned tissue (as described in the laboratory manual) with an associated pathology report. Acceptable samples may also include core-needle biopsies for deep tumor tissue (minimum of five cores), excisional, incisional, punch, or forceps biopsies for cutaneous, subcutaneous, or mucosal lesions. Patients with less than five cores may be considered eligible with approval from the Medical Monitor. Fine-needle aspiration samples, brushings, cell pellets from effusions or ascites, and lavage samples are not acceptable. Tumor tissue from bone metastases is difficult to evaluate for PD-L1 expression and should be avoided. However if a bony metastatic site is the only viable source of tissue, it may be an acceptable tumor specimen with Medical Monitor approval. Bony tissue that has been decalcified may be acceptable prior to decalcification, as many reagents have strong acids that damage antigens used for PD-L1 IHC and nucleic acid for sequencing. If adequate tissue from distinct timepoints (such as time of initial diagnosis and time of disease recurrence) and/or multiple metastatic tumors are available, priority should be given to the tissue most recently collected (ideally subsequent to the most recent systemic adjuvant therapy). Multiple samples may be collected for a given patient, on the basis of availability; however, the requirement for a block or sectioned tissue should be satisfied by a single biopsy or resection specimen. A patient with insufficient or unavailable archival tissue will not be eligible due to the need for evaluable tumor tissue to create the PCV unless the patient is willing to consent to and undergo a pretreatment biopsy sample collection of the tumor (refer to above for acceptable samples).
Enrollment is limited to patients with at least five identified tumor neoantigens and adequate tumor material (both quality and quantity) to allow manufacture of vaccine, as defined by the Sponsor. Archival tumor tissue is acceptable for CIT-naive patients; it must be submitted and assessed for evaluation of mutations prior to enrollment. A baseline tumor biopsy is required for CIT-experienced patients (i.e., patients who received treatment with an immune checkpoint inhibitor in the adjuvant setting) and must be submitted and assessed for evaluation of mutations prior to enrollment. CIT-experienced patients who have undergone a tumor biopsy after receiving CIT, but prior to enrollment may use that tissue for screening if sufficient material exists. If available, patients should also submit archival tumor tissue for evaluation. Archival tissue may also be used for CIT-experienced patients, in case the baseline fresh tumor biopsy is inadequate for manufacturing. Patients whose tumor tissue is unevaluable or who have an insufficient number of mutations to manufacture vaccine are not eligible.
For women of childbearing potential: agreement to remain abstinent (refrain from heterosexual intercourse) or use contraceptive measures, and agreement to refrain from donating eggs
For men: agreement to remain abstinent (refrain from heterosexual intercourse) or use a condom, and agreement to refrain from donating sperm

Patients who meet any of the following criteria are excluded from study entry:
Ocular or acral melanoma
Pregnant or breastfeeding, or intending to become pregnant during the study or within 1 month after the final dose of RO7198457 or 4 months after the final dose of pembrolizumab, whichever occurs later. Women of childbearing potential (including women who have had a tubal ligation) must have a negative serum pregnancy test result within 14 days prior to initiation of study drug (i.e., Cycle 1, Day 1).
Significant cardiovascular disease, such as New York Heart Association cardiac disease (Class II or greater), myocardial infarction within the previous 3 months, unstable arrhythmias, and/or unstable angina.
Known clinically significant liver disease, including active viral, alcoholic, or other hepatitis, cirrhosis, and inherited liver disease or current alcohol abuse
Major surgical procedure within 28 days prior to Cycle 1, Day 1, or anticipation of need for a major surgical procedure during the course of the study
Any other diseases, metabolic dysfunction, physical examination finding, and/or clinical laboratory finding giving reasonable suspicion of a disease or condition that contraindicates the use of an investigational drug or that may affect the interpretation of the results or may render the patient at high risk from treatment complications
Corticosteroids at dosages higher than 7.5 mg prednisolone (if not for physiologic substitution)
Previous splenectomy
Known primary immunodeficiencies, either cellular (e.g., DiGeorge syndrome, T-negative severe combined immunodeficiency [SCID]) or combined T- and B-cell immunodeficiencies (e.g., T- and B-negative SCID, Wiskott-Aldrich syndrome, ataxia telangiectasia, common variable immunodeficiency)
Symptomatic, untreated, or actively progressing CNS metastases. Patients with a history of CNS lesions are eligible, provided that all of the following criteria are met:
   Measurable disease, per RECIST v1.1, must be present outside the CNS
   Only supratentorial and cerebellar metastases allowed (i.e., no metastases to midbrain, pons, medulla or spinal cord)
   History of metastases within 10 mm of the optic apparatus (optic nerves and chiasm)
   No ongoing requirement for corticosteroids as therapy for CNS disease
   No stereotactic radiation within 7 days
   No prior whole-brain radiation
   No clinical evidence of interim progression between the completion of CNS-directed therapy and the screening radiographic study
   Patients with new asymptomatic CNS metastases detected at the screening scan must receive radiation therapy and/or surgery for CNS metastases. Following treatment, these patients may then be eligible without the need for an additional brain scan prior to Cycle 1 Day 1, if all other criteria are met
   Treatment with an anticonvulsant at a stable dose is allowed
   No history of intracranial hemorrhage from CNS lesions
History of leptomeningeal metastatic disease
Uncontrolled tumor-related pain. Patients requiring narcotic pain medication must be on a stable regimen at study entry. Symptomatic lesions amenable to palliative radiotherapy (e.g., bone metastases or metastases causing nerve impingement) should be treated prior to enrollment. Patients should be recovered from the effects of radiation. There is no required minimum recovery period. Asymptomatic metastatic lesions that would likely cause functional deficits or intractable pain with further growth (e.g., epidural metastasis that is not currently associated with spinal cord compression) should be considered for loco-regional therapy if appropriate prior to enrollment.
Uncontrolled pleural effusion, pericardial effusion, or ascites requiring repeated drainage more than once every 28 days. Indwelling drainage catheters (e.g., PleurX®) are allowed.
Any anti-cancer therapy, in the metastatic setting whether investigational or approved, including chemotherapy, hormonal therapy, and/or radiotherapy, prior to initiation of study treatment, with the following exceptions:
   Herbal therapy >1 week before Cycle 1, Day 1
   Palliative radiotherapy for painful metastases or metastases in potentially sensitive locations (e.g., epidural space) >2 weeks prior to Cycle 1, Day 1
   Prior cancer vaccines (e.g., T-vec) are not allowed
Malignancies other than disease under study within 5 years prior to Cycle 1, Day 1, with the exception of those with a negligible risk of metastasis or death (such as adequately treated carcinoma in situ of the cervix, basal or squamous cell skin cancer, localized prostate cancer, or ductal carcinoma in situ)
Uncontrolled hypercalcemia (>1.5 mmol/L ionized calcium or Ca⁺²>12 mg/dL or corrected serum calcium ≥ULN) or symptomatic hypercalcemia requiring continued use of bisphosphonate therapy. Patients who are receiving bisphosphonate therapy or denosumab specifically to prevent skeletal events and who do not have a history of clinically significant hypercalcemia are eligible.
Spinal cord compression not definitively treated with surgery and/or radiation or previously diagnosed and treated spinal cord compression without evidence that disease has been clinically stable for 2 weeks prior to screening.
History of autoimmune disease, including, but not limited to, systemic lupus erythematosus, rheumatoid arthritis, inflammatory bowel disease, vascular thrombosis associated with antiphospholipid syndrome, Wegener granulomatosis, Sjögren syndrome, Bell palsy, Guillain-Barré syndrome, multiple sclerosis, vasculitis, or glomerulonephritis with the following exceptions:
   Patients with a history of autoimmune hypothyroidism on a stable dose of thyroid replacement hormone may be eligible.
   Patients with controlled type 1 diabetes mellitus on a stable insulin regimen may be eligible.
   Patients with eczema, psoriasis, lichen simplex chronicus, or vitiligo with dermatologic manifestations only (e.g., no psoriatic arthritis) may be eligible provided that they meet the following conditions:
      Rash must cover less than 10% of the body surface area
      Disease is well controlled at baseline and only requires low potency topical steroids
      There are no acute exacerbations of underlying condition within the last 12 months (e.g., not requiring psoralen plus ultraviolet A radiation, methotrexate, retinoids, biologic agents, oral calcineurin inhibitors, high potency, or oral steroids)
Treatment with monoamine oxidase inhibitors (MAOIs) within 3 weeks prior to Cycle 1, Day 1
Treatment with systemic immunosuppressive medications (including, but not limited to, prednisone ≥7.5 mg/day, cyclophosphamide, azathioprine, methotrexate, thalidomide, and TNF-α antagonists) within 2 weeks prior to Cycle 1, Day 1
   Patients who have received acute, low-dose, systemic immunosuppressant medications (e.g., a one-time dose of dexamethasone for nausea) may be enrolled in the study after discussion with and approval by the Medical Monitor
   The use of inhaled corticosteroids (e.g., fluticasone for chronic obstructive pulmonary disease) is allowed
   The use of oral mineralocorticoids (e.g., fludrocortisone for patients with orthostatic hypotension) is allowed
   Physiologic doses of corticosteroids for adrenal insufficiency are allowed
History of idiopathic pulmonary fibrosis, pneumonitis (including drug induced), organizing pneumonia (i.e., bronchiolitis obliterans, cryptogenic organizing pneumonia, etc.), or evidence of active pneumonitis on screening chest computed tomography (CT) scan. History of radiation pneumonitis in the radiation field (fibrosis) is permitted.
Positive test for HIV infection
Active hepatitis B (defined as having a positive hepatitis B surface antigen [HBsAg] test at screening) Patients with past or resolved hepatitis B infection (defined as having a negative HBsAg test and a positive IgG antibody to hepatitis B core antigen [anti-HBc]) are eligible. HBV DNA must be obtained in these patients prior to Cycle 1, Day 1 and must demonstrate no active infection.
Active hepatitis C. Patients positive for HCV antibody are eligible only if polymerase chain reaction (PCR) is negative for HCV RNA.
Known active or latent tuberculosis infection. If the investigator considers a potential patient to be at an increased risk for infection with Mycobacterium tuberculosis, latent tuberculosis diagnostic procedures must be followed according to local practice standards during the screening period
Severe infections within 4 weeks prior to Cycle 1, Day 1 including, but not limited to, hospitalization for complications of infection, bacteremia, or severe pneumonia
Recent infections not meeting the criteria for severe infections, including the following:
   Signs or symptoms of infection within 2 weeks prior to Cycle 1, Day 1
   Received oral or IV antibiotics within 2 weeks prior to Cycle 1, Day 1
   Patients receiving prophylactic antibiotics (e.g., for prevention of a urinary tract infection or chronic obstructive pulmonary disease) are eligible
Prior allogeneic bone marrow transplantation or prior solid organ transplantation
Administration of a live, attenuated vaccine within 4 weeks before Cycle 1, Day 1 or anticipation that such a live, attenuated vaccine is required during the study. Influenza vaccination should be given during influenza season only. Patients must not receive live, attenuated influenza vaccine (e.g., FluMist®) within 4 weeks prior to Cycle 1, Day 1 or at any time during the study, and for 5 months following the last study treatment.
Known hypersensitivity to the active substance or to any of the excipients in the vaccine
History of severe allergic, anaphylactic, or other hypersensitivity reactions to chimeric or humanized antibodies or fusion proteins
Known hypersensitivity to Chinese hamster ovary-cell products
Allergy or hypersensitivity to components of the pembrolizumab formulation

### Example 2

This example describes an exemplary RNA vaccine to be used in the methods described herein.

### Overall Description

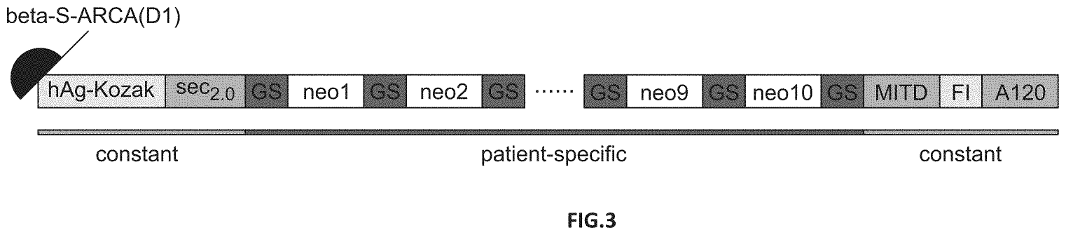

The RNA vaccine is a single-stranded messenger ribonucleic acid (mRNA) molecule that encodes constant sequences and patient-specific tumor neoantigen sequences. Specifically, it is a 5′-capped, single-stranded messenger RNA (mRNA). Each mRNA encodes up to 20 neoepitopes defined by the patient's tumor-specific mutations that have been identified and selected. The sequences containing patient tumor-specific mutations are typically composed of 81 nucleotides. Shown in FIG. 3 is a schematic presentation of the mRNA (in this example, an mRNA encoding 10 patient-specific neoepitopes).

The constant sequence elements include the following: 5′ cap (beta-S-ARCA), 5′-, 3′-untranslated regions [UTR], secretory signal peptide [sec_{2.0}], MHC [major histocompatibility complex] class I transmembrane and cytoplasmic domains [MITD], and poly(A)-tail. These constant sequences have been optimized for translational efficiency and stability of the mRNA, and are identical for each batch, and are thus identical for all patients. The roles of all constant sequence elements are summarized in Table 4; they flank the patient-specific neoepitope regions and glycine/serine (GS)-rich linkers.

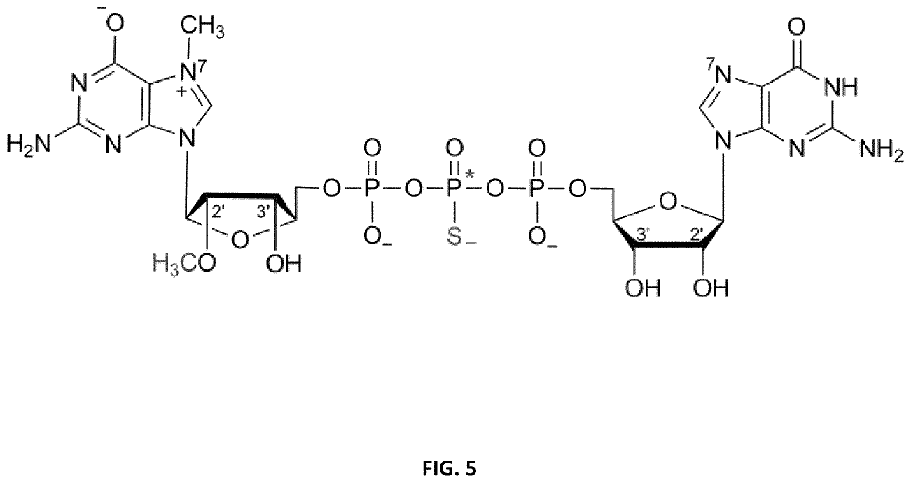

TABLE 4

| Element | Description |
|---|---|
| 5′-cap | Beta-S-ARCA(D1) (see FIG. 5) is utilized as a specific capping structure at the |
| | 5′-end of the RNA cancer vaccine for improved RNA stability and translational |
| | efficiency (Kuhn et al. 2010). |
| 5′-UTR | The 5′-UTR sequence has been derived from the human alpha-globin RNA. An |
| (hAg-Kozak) | optimized “Kozak sequence” has been added in order to increase translational |
| | efficiency (Kozak 1987). |
| Secretory | The secretory signal peptide “sec_{2.0}” derived from the sequence encoding the |
| signal peptide | human MHC Class I complex alpha chain “HLA-I, Cw*” is used as a |
| (sec_{2.0}) | fusion-protein tag to improve antigen processing and presentation (Kreiter et al. |
| | 2008). “HLA-I, Cw*” was chosen, because it corresponds to one of the most |
| | frequent haplotypes and has a high homology to other frequent MHC Class I |
| | alleles. |
| MITD | MITD corresponds to the transmembrane and cytoplasmic domains of the MHC |
| | class I molecule and is used as a fusion-protein tag to improve antigen |
| | processing and presentation (Kreiter et al. 2008). |
| 3′-UTR (Fl) | The 3′-UTR is a combination of two sequence elements derived from the AES |
| | mRNA (called F) and the mitochondrial encoded 12S ribosomal RNA (called I). |
| | These were identified by performing an ex vivo selection process for sequences |
| | that confer RNA stability. |
| poly(A)-tail | A poly(A)-tail measuring 120 nucleotides (A 120) is added to ensure high RNA |
| | stability and protein expression (Holtkamp et al. 2006). |

Abbreviations:
AES = amino terminal enhancer of split;
MHC = major histocompatibility complex;
MITD = MHC class I transmembrane and cytoplasmic domains;
UTR = untranslated region.

### Constant Sequence Description

RNA[1,2-[m₂⁷.^{2′}.^{O}G-(5′→55′)-ppₛp-G (Rp-isomer)]] (constant 5′ UTR plus sec_{2.0} linked to constant MITD plus 3′ UTR and poly(A)-tail)

Sequence length: 739 nucleotides (A: 255, C: 204, G: 168, U: 112)

Shown in FIG. 4 is the RNA sequence of the constant region of the exemplary RNA vaccine. The insertion site for patient-specific sequences (C131-A132) is depicted in bold text. See Table 5 for the modified bases and uncommon links in the RNA sequence.

TABLE 5

| Type | Location | Description |
|---|---|---|
| Modified Base | G1 | m₂^{7·2′·O} G |
| Uncommon Link | G1-G2 | (5′→ 5′)-ppₛp- |
| Uncommon Link | C131- | Insertion site for |
| | A132 | patient-specific sequences |

Altogether, the length of each RNA has a range of approximately 1000-2000 nucleotides, depending on the size of each neoepitope and the number of neoepitopes encoded on each RNA. The constant regions of the RNA, independent of patient-specific sequences, constitute 739 ribonucleotides.

### REFERENCES

Holtkamp S, Kreiter S, Selmi A, et al. Modification of antigen-encoding RNA increases stability, translational efficacy, and T-cell stimulatory capacity of dendritic cells. Blood 2006; 108:4009-17
Kozak M. At least six nucleotides preceding the AUG initiator codon enhance translation in mammalian cells. J Mol Biol 1987; 196:947-50.
Kreiter S, Selmi A, Diken M, et al. Increased antigen presentation efficiency by coupling antigens to MHC class I trafficking signals. J Immunol 2008; 180:309-18.
Kuhn A N, Diken M, Kreiter S, et al. Phosphorothioate cap analogs increase stability and translational efficiency of RNA vaccines in immature dendritic cells and induce superior immune responses in vivo. Gene Ther 2010; 17:961-71.
Trinh R, Gurbaxani B, Morrison S L, et al. Optimization of codon pair use within the (GGGGS)3 linker sequence results in enhanced protein expression. Mol Immunol 2004; 40:717-22.

SEQUENCES

All polynucleotide sequences are depicted in the 5′→3′ direction. All polypeptide sequences are depicted in the N-terminal to C-terminal direction.

Anti-PDL1 antibody HVR-H1 sequence
(SEQ ID NO: 1)
GFTFSDSWIH
Anti-PDL1 antibody HVR-H2 sequence
(SEQ ID NO: 2)
AWISPYGGSTYYADSVKG
Anti-PDL1 antibody HVR-H3 sequence
(SEQ ID NO: 3)
RHWPGGFDY
Anti-PDL1 antibody HVR-L1 sequence
(SEQ ID NO: 4)
RASQDVSTAVA
Anti-PDL1 antibody HVR-L2 sequence
(SEQ ID NO: 5)
SASFLYS
Anti-PDL1 antibody HVR-L3 sequence
(SEQ ID NO: 6)
QQYLYHPAT
Anti-PDL1 antibody VH sequence
(SEQ ID NO: 7)
EVQLVESGGGLVQPGGSLRLSCAASGFTFSDSWIHWVRQAPGKGLEWVAWISPYGGSTYYADS
VKGRFTISADTSKNTAYLQMNSLRAEDTAVYYCARRHWPGGFDYWGQGTLVTVSS
Anti-PDL1 antibody VL sequence
(SEQ ID NO: 8)
DIQMTQSPSSLSASVGDRVTITCRASQDVSTAVAWYQQKPGKAPKLLIYSASF
LYSGVPSRFSGSGSGTDFTLTISSLQPEDFATYYCQQYLYHPATFGQGTKVEIKR
Anti-PDL1 antibody heavy chain sequence
(SEQ ID NO: 9)
EVQLVESGGGLVQPGGSLRLSCAASGFTFSDSWIHWVRQAPGKGLEWVAWISPYGGSTYYADS
VKGRFTISADTSKNTAYLQMNSLRAEDTAVYYCARRHWPGGFDYWGQGTLVTVSSASTKGPSV
FPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYASTYRVVSVLTVLHQDWLNGKEY
KCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPG
Anti-PDL1 antibody light chain sequence
(SEQ ID NO: 10)
DIQMTQSPSSLSASVGDRVTITCRASQDVSTAVAWYQQKPGKAPKWYSASFLYSGVPSRFSGS
GSGTDFTLTISSLQPEDFATYYCQQYLYHPATFGQGTKVEIKRTVAAPSVFIFPPSDEQLKSGTAS
VVCLLNNFYPREAKVQWKVDNALQSGNSQESVTEQDSKDSTYSLSSTLTLSKADYEKHKVYAC
EVTHQGLSSPVTKSFNRGEC
Nivolumab heavy chain sequence
(SEQ ID NO: 11)
QVQLVESGGGVVQPGRSLRLDCKASGITFSNSGMEIWVRQAPGKGLEWVAVIWY
DGSKRYYADSVKGRFTISRDNSKNTLFLQMNSLRAEDTAVYYCATNDDYWGQGTLVTVSSAST
KGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSLG
Nivolumab light chain sequence
(SEQ ID NO: 12)
EIVLTQSPATLSLSPGERATLSCRASQSVSSYLAWYQQKPGQAPRLLIYDASNRAT
GIPARFSGSGSGTDFTLTISSLEPEDFAVYYCQQSSNWPRTFGQGTKVEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGEC
Pembrolizumab heavy chain sequence
(SEQ ID NO: 13)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRDYRFDMGFDYW
GQGTTVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
Pembrolizumab light chain sequence
(SEQ ID NO: 14)
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKRTVAAPSVF
IFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Avelumab heavy chain sequence
(SEQ ID NO: 15)
EVQLLESGGGLVQPGGSLRLSCAASGFTFSSYIMMWVRQAPGKGLEWVSSIYPSGGITFYADTV
KGRFTISRDNSKNTLYLQMNSLRAEDTAVYYCARIKLGTVTIVDYWGQGTLVTVSSASTKGPSV
FPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
Avelumab light chain sequence
(SEQ ID NO: 16)
QSALTQPASVSGSPGQSITISCTGTSSDVGGYNYVSWYQQHPGKAPKLMIYDVSNRPSGVSNRFS
GSKSGNTASLTISGLQAEDEADYYCSSYTSSSTRVFGTGTKVTVLGQPKANPTVTLFPPSSEELQA
NKATLVCLISDFYPGAVTVAWKADGSPVKAGVETTKPSKQSNNKYAASSYLSLTPEQWKSHRS
YSCQVTHEGSTVEKTVAPTECS
Durvalumab heavy chain sequence
(SEQ ID NO: 17)
EVQLVESGGGLVQPGGSLRLSCAASGFTFSRYWMSWVRQAPGKGLEWVANIKQDGSEKYYVD
SVKGRFTISRDNAKNSLYLQMNSLRAEDTAVYYCAREGGWFGELAFDYWGQGTLVTVSSASTK
GPSVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPEFEGGPSVFLFPPKPKDTLM
ISRTPEVTCVVVDVSHEDPEVKFNWYVDGVEVHNAKTKPREEQYNSTYRVVSVLTVLHQDWLN
GKEYKCKVSNKALPASIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSL
SPG
Durvalumab light chain sequence
(SEQ ID NO: 18)
EIVLTQSPGTLSLSPGERATLSCRASQRVSSSYLAWYQQKPGQAPRLLIYDASSRATGIPDRFSGS
GSGTDFTLTISRLEPEDFAVYYCQQYGSLPWTFGQGTKVEIKRTVAAPSVFIFPPSDEQLKSGTAS
VVCLLNNFYPREAKVQWKVDNALQSGNSQESVTEQDSKDSTYSLSSTLTLSKADYEKHKVYAC
EVTHQGLSSPVTKSFNRGEC
Full PCV RNA 5′ constant sequence
(SEQ ID NO: 19)
GGCGAACUAGUAUUCUUCUGGUCCCCACAGACUCAGAGAGAACCCGCCACCAUGAGAGUG
AUGGCCCCCAGAACCCUGAUCCUGCUGCUGUCUGGCGCCCUGGCCCUGACAGAGACAUGG
GCCGGAAGC
Full PCV RNA 3′ constant sequence
(SEQ ID NO: 20)
AUCGUGGGAAUUGUGGCAGGACUGGCAGUGCUGGCCGUGGUGGUGAUCGGAGCCGUGGU
GGCUACCGUGAUGUGCAGACGGAAGUCCAGCGGAGGCAAGGGCGGCAGCUACAGCCAGGC
CGCCAGCUCUGAUAGCGCCCAGGGCAGCGACGUGUCACUGACAGCCUAGUAACUCGAGCU
GGUACUGCAUGCACGCAAUGCUAGCUGCCCCUUUCCCGUCCUGGGUACCCCGAGUCUCCC
CCGACCUCGGGUCCCAGGUAUGCUCCCACCUCCACCUGCCCCACUCACCACCUCUGCUAGU
UCCAGACACCUCCCAAGCACGCAGCAAUGCAGCUCAAAACGCUUAGCCUAGCCACACCCC
CACGGGAAACAGCAGUGAUUAACCUUUAGCAAUAAACGAAAGUUUAACUAAGCUAUACU
AACCCCAGGGUUGGUCAAUUUCGUGCCAGCCACACCGAGACCUGGUCCAGAGUCGCUAGC
CGCGUCGCU
Full PCV Kozak RNA
(SEQ ID NO: 21)
GGCGAACUAGUAUUCUUCUGGUCCCCACAGACUCAGAGAGAACCCGCCACC
Full PCV Kozak DNA
(SEQ ID NO: 22)
GGCGAACTAGTATTCTTCTGGICCCCACAGACTCAGAGAGAACCCGCCACC
short Kozak RNA
(SEQ ID NO: 23)
UUCUUCUGGUCCCCACAGACUCAGAGAGAACCCGCCACC
short Kozak DNA
(SEQ ID NO: 24)
TTCTTCTGGTCCCCACAGACTCAGAGAGAACCCGCCACC
sec RNA
(SEQ ID NO: 25)
AUGAGAGUGAUGGCCCCCAGAACCCUGAUCCUGCUGCUGUCUGGCGCCCUGGCCCUGACA
GAGACAUGGGCCGGAAGC
sec DNA
(SEQ ID NO: 26)
ATGAGAGTGATGGCCCCCAGAACCCTGATCCTGCTGCTGTCTGGCGCCCTGGCCCTGACAGA
GACATGGGCCGGAAGC
sec protein
(SEQ ID NO: 27)
MRVMAPRTLILLLSGALALTETWAGS
MITD RNA
(SEQ ID NO: 28)
AUCGUGGGAAUUGUGGCAGGACUGGCAGUGCUGGCCGUGGUGGUGAUCGGAGCCGUGGU
GGCUACCGUGAUGUGCAGACGGAAGUCCAGCGGAGGCAAGGGCGGCAGCUACAGCCAGGC
CGCCAGCUCUGAUAGCGCCCAGGGCAGCGACGUGUCACUGACAGCC
MITD DNA
(SEQ ID NO: 29)
ATCGTGGGAATTGTGGCAGGACTGGCAGTGCTGGCCGTGGTGGTGATCGGAGCCGTGGTGG
CTACCGTGATGTGCAGACGGAAGTCCAGCGGAGGCAAGGGCGGCAGCTACAGCCAGGCCGC
CAGCTCTGATAGCGCCCAGGGCAGCGACGTGTCACTGACAGCC
MITD protein
(SEQ ID NO: 30)
IVGIVAGLAVLAVVVIGAVVATVMCRRKSSGGKGGSYSQAASSDSAQGSDVSLTA
Full PCV FI RNA
(SEQ ID NO: 31)
CUCGAGCUGGUACUGCAUGCACGCAAUGCUAGCUGCCCCUUUCCCGUCCUGGGUACCCCG
AGUCUCCCCCGACCUCGGGUCCCAGGUAUGCUCCCACCUCCACCUGCCCCACUCACCACCU
CUGCUAGUUCCAGACACCUCCCAAGCACGCAGCAAUGCAGCUCAAAACGCUUAGCCUAGC
CACACCCCCACGGGAAACAGCAGUGAUUAACCUUUAGCAAUAAACGAAAGUUUAACUAAG
CUAUACUAACCCCAGGGUUGGUCAAUUUCGUGCCAGCCACACCGAGACCUGGUCCAGAGU
CGCUAGCCGCGUCGCU
Full PCV FI DNA
(SEQ ID NO: 32)
CTGGTACTGCATGCACGCAATGCTAGCTGCCCCTTTCCCGTCCTGGGTACCCCGAGTCTCCCC
CGACCTCGGGTCCCAGGTATGCTCCCACCTCCACCTGCCCCACTCACCACCTCTGCTAGTTCC
AGACACCTCCCAAGCACGCAGCAATGCAGCTCAAAACGCTTAGCCTAGCCACACCCCCACG
GGAAACAGCAGTGATTAACCTTTAGCAATAAACGAAAGTTTAACTAAGCTATACTAACCCCA
GGGTTGGTCAATTTCGTGCCAGCCACACCGAGACCTGGTCCAGAGTCGCIAGCCGCGTCGCT
F element RNA
(SEQ ID NO: 33)
CUGGUACUGCAUGCACGCAAUGCUAGCUGCCCCUUUCCCGUCCUGGGUACCCCGAGUCUC
CCCCGACCUCGGGUCCCAGGUAUGCUCCCACCUCCACCUGCCCCACUCACCACCUCUGCUA
GUUCCAGACACCUCC
F element DNA
(SEQ ID NO: 34)
CTGGTACTGCATGCACGCAATGCTAGCTGCCCCTTTCCCGTCCTGGGTACCCCGAGTCTCCCC
CGACCTCGGGTCCCAGGTATGCTCCCACCTCCACCTGCCCCACTCACCACCTCTGCTAGTTCC
AGACACCTCC
I element RNA
(SEQ ID NO: 35)
CAAGCACGCAGCAAUGCAGCUCAAAACGCUUAGCCUAGCCACACCCCCACGGGAAACAGC
AGUGAUUAACCUUUAGCAAUAAACGAAAGUUUAACUAAGCUAUACUAACCCCAGGGUUG
GUCAAUUUCGUGCCAGCCACACCG
I element DNA
(SEQ ID NO: 36)
CAAGCACGCAGCAATGCAGCTCAAAACGCTTAGCCTAGCCACACCCCCACGGGAAACAGCA
GTGATTAACCTTTAGCAATAAACGAAAGTTTAACTAAGCTATACTAACCCCAGGGTTGGTCA
ATTTCGTGCCAGCCACACCG
linker RNA
(SEQ ID NO: 37)
GGCGGCUCUGGAGGAGGCGGCUCCGGAGGC
linker DNA
(SEQ ID NO: 38)
GGCGGCTCTGGAGGAGGCGGCTCCGGAGGC
linker protein
(SEQ ID NO: 39)
GGSGGGGSGG
Full PCV DNA 5′ constant sequence
(SEQ ID NO: 40)
GGCGAACTAGTATTCTTCTGGTCCCCACAGACTCAGAGAGAACCCGCCACCATGAGAGTGAT
GGCCCCCAGAACCCTGATCCTGCTGCTGTCTGGCGCCCTGGCCCTGACAGAGACATGGGCCG
GAAGC
Full PCV DNA 3′ constant sequence
(SEQ ID NO: 41)
ATCGTGGGAATTGTGGCAGGACTGGCAGTGCTGGCCGTGGTGGTGATCGGAGCCGTGGTGG
CTACCGTGATGTGCAGACGGAAGTCCAGCGGAGGCAAGGGCGGCAGCTACAGCCAGGCCGC
CAGCTCTGATAGCGCCCAGGGCAGCGACGTGTCACTGACAGCCTAGTAACTCGAGCTGGTAC
TGCATGCACGCAATGCTAGCTGCCCCTTTCCCGTCCTGGGTACCCCGAGTCTCCCCCGACCTC
GGGTCCCAGGTATGCTCCCACCTCCACCTGCCCCACTCACCACCTCTGCTAGTTCCAGACACC
TCCCAAGCACGCAGCAATGCAGCTCAAAACGCTTAGCCTAGCCACACCCCCACGGGAAACA
GCAGTGATTAACCTTTAGCAATAAACGAAAGTTTAACTAAGCTATACTAACCCCAGGGTTGG
TCAATTTCGTGCCAGCCACACCGAGACCTGGTCCAGAGTCGCTAGCCGCGTCGCT
Full PCV RNA with 5′ GG from cap
(SEQ ID NO: 42)
GGGGCGAACU AGUAUUCUUC UGGUCCCCAC AGACUCAGAG AGAACCCGCC
ACCAUGAGAG UGAUGGCCCC CAGAACCCUG AUCCUGCUGC UGUCUGGCGC
CCUGGCCCUG ACAGAGACAU GGGCCGGAAG CNAUCGUGGGA AUUGUGGCAG
GACUGGCAGU GCUGGCCGUG GUGGUGAUCG GAGCCGUGGU GGCUACCGUG
AUGUGCAGAC GGAAGUCCAG CGGAGGCAAG GGCGCCAGCU ACAGCCAGGC
CGCCAGCUCU GAUAGCGCCC AGGGCAGCGA CGUGUCACUG ACAGCCUAGU
AACUCGAGCU GGUACUGCAU GCACGCAAUG CUAGCUGCCC CUUUCCCGUC
CUGGGUACCC CGAGUCUCCC CCGACCUCGG GUCCCAGGUA UGCUCCCACC
UCCACCUGCC CCACUCACCA CCUCUGCUAG UUCCAGACAC CUCCCAAGCA
CGCAGCAAUG CAGCUCAAAA CGCUUAGCCU AGCCACACCC CCACGGGAAA
CAGCAGUGAU UAACCUUUAG CANUAAACGA AAGUUUAACU AAGCTATACT
AACCCCAGGG UUGGUCAAUU UCGUGCCAGC CACACCGAGA CCUGGUCCAG
AGUCGCUAGC CGCGUCGCUA AAAAAAAAAA AAAAAAAAAA AAAAAAAAAA
AAAAAAAAAA AAAAAAAAAA AAAAAAAAAA AAAAAAAAAA AAAAAAAAAA
AAAAAAAAAA AAAAAAAAAA AAAAAAAAAA AAAAAAAAA

What is claimed is:

1. A method of treating or delaying progression of cancer in an individual, comprising administering to the individual an effective amount of a PD-1 axis binding antagonist and an RNA vaccine, wherein the RNA vaccine comprises one or more polynucleotides encoding one or more neoepitopes resulting from cancer-specific somatic mutations present in a tumor specimen obtained from the individual.

2. The method of claim 1, wherein the PD-1 axis binding antagonist is a PD-1 binding antagonist.

3. The method of claim 2, wherein the PD-1 binding antagonist is an anti-PD-1 antibody.

4. The method of claim 3, wherein the anti-PD-1 antibody is nivolumab or pembrolizumab.

5. The method of claim 3 or claim 4, wherein the anti-PD-1 antibody is administered to the individual at a dose of about 200 mg.

6. The method of claim 1, wherein the PD-1 axis binding antagonist is a PD-L1 binding antagonist.

7. The method of claim 6, wherein the PD-L1 binding antagonist is an anti-PD-L1 antibody.

8. The method of claim 7, wherein the anti-PD-L1 antibody is avelumab or durvalumab.

9. The method of claim 7, wherein the anti-PD-L1 antibody comprises:
(a) a heavy chain variable region (VH) that comprises an HVR-H1 comprising an amino acid sequence of GFTFSDSWIH (SEQ ID NO:1), an HVR-2 comprising an amino acid sequence of AWISPYGGSTYYADSVKG (SEQ ID NO:2), and HVR-3 comprising an amino acid RHWPGGFDY (SEQ ID NO:3), and
(b) a light chain variable region (VL) that comprises an HVR-L1 comprising an amino acid sequence of RASQDVSTAVA (SEQ ID NO:4), an HVR-L2 comprising an amino acid sequence of SASFLYS (SEQ ID NO:5), and an HVR-L3 comprising an amino acid sequence of QQYLYHPAT (SEQ ID NO:6).

10. The method of claim 7, wherein the anti-PD-L1 antibody comprises a heavy chain variable region (V_{H}) comprising an amino acid sequence of SEQ ID NO:7 and a light chain variable region (V_{L}) comprising an amino acid sequence of SEQ ID NO:8.

11. The method of claim 7, wherein the anti-PD-L1 antibody is atezolizumab.

12. The method of any one of claims 7-11, wherein the anti-PD-L1 antibody is administered to the individual at a dose of about 1200 mg.

13. The method of any one of claims 1-12, wherein the PD-1 axis binding antagonist is administered to the individual at an interval of 21 days or 3 weeks.

14. The method of any one of claims 1-13, wherein the RNA vaccine comprises one or more polynucleotides encoding 10-20 neoepitopes resulting from cancer-specific somatic mutations present in the tumor specimen.

15. The method of any one of claims 1-14, wherein the RNA vaccine is formulated in a lipoplex nanoparticle or liposome.

16. The method of any one of claims 1-15, wherein the RNA vaccine is administered to the individual at a dose of about 15 μg, about 25 μg, about 38 μg, about 50 μg, or about 100 μg.

17. The method of any one of claims 1-16, wherein the RNA vaccine is administered to the individual at an interval of 21 days or 3 weeks.

18. The method of any one of claims 1-16, wherein the PD-1 axis binding antagonist and the RNA vaccine are administered to the individual in 8 21-day Cycles, and wherein the RNA vaccine is administered to the individual on Days 1, 8, and 15 of Cycle 2 and Day 1 of Cycles 3-7.

19. The method of claim 18, wherein the PD-1 axis binding antagonist is administered to the individual on Day 1 of Cycles 1-8.

20. The method of claim 18 or claim 19, wherein the PD-1 axis binding antagonist and the RNA vaccine are further administered to the individual after Cycle 8.

21. The method of claim 20, wherein the PD-1 axis binding antagonist and the RNA vaccine are further administered to the individual in 17 additional 21-day Cycles, wherein the PD-1 axis binding antagonist is administered to the individual on Day 1 of Cycles 13-29, and wherein the RNA vaccine is administered to the individual on Day 1 of Cycles 13, 21, and 29.

22. The method of claim 1, wherein the PD-1 axis binding antagonist and the RNA vaccine are administered to the individual in 8 21-day Cycles, wherein the PD-1 axis binding antagonist is pembrolizumab and is administered to the individual at a dose of about 200 mg on Day 1 of Cycles 1-8, and wherein the RNA vaccine is administered to the individual at a dose of about 25 μg on Days 1, 8, and 15 of Cycle 2 and Day 1 of Cycles 3-7.

23. The method of claim 22, wherein the RNA vaccine is administered to the individual at doses of about 25 μg on Day 1 of Cycle 2, about 25 μg on Day 8 of Cycle 2, about 25 μg on Day 15 of Cycle 2, and about 25 μg on Day 1 of each of Cycles 3-7.

24. The method of any one of claims 1-23, wherein the PD-1 axis binding antagonist and the RNA vaccine are administered intravenously.

25. The method of any one of claims 1-24, wherein the individual is a human.

26. The method of any one of claims 1-25, wherein the cancer is selected from the group consisting of non-small cell lung cancer, bladder cancer, colorectal cancer, triple negative breast cancer, renal cancer, and head and neck cancer.

27. The method of any one of claims 1-25, wherein the cancer is melanoma.

28. The method of claim 27, wherein the melanoma is cutaneous or mucosal melanoma.

29. The method of claim 27, wherein the melanoma is not ocular or acral melanoma.

30. The method of any one of claims 27-29, wherein the melanoma is metastatic or unresectable locally advanced melanoma.

31. The method of claim 30, wherein the melanoma is stage IV melanoma.

32. The method of claim 30, wherein the melanoma is stage IIIC or stage IIID melanoma.

33. The method of claim 27, wherein the melanoma is previously untreated advanced melanoma.

34. The method of any one of claims 1-33, wherein the method results in improved progression-free survival (PFS).

35. The method of any one of claims 1-34, wherein the method results in increased objective response rate (ORR).

36. A kit comprising a PD-1 axis binding antagonist for use in combination with an RNA vaccine for treating an individual having cancer according to a method of any one of claims 1-35.

37. A PD-1 axis binding antagonist for use in a method of treating a human individual having cancer, the method comprising administering to the individual an effective amount of the PD-1 axis binding antagonist in combination with an RNA vaccine, wherein the RNA vaccine comprises one or more polynucleotides encoding one or more neoepitopes resulting from cancer-specific somatic mutations present in a tumor specimen obtained from the individual.

38. An RNA vaccine for use in a method of treating a human individual having cancer, the method comprising administering to the individual an effective amount of the RNA vaccine in combination with a PD-1 axis binding antagonist, wherein the RNA vaccine comprises one or more polynucleotides encoding one or more neoepitopes resulting from cancer-specific somatic mutations present in a tumor specimen obtained from the individual.

39. An RNA molecule comprising, in the 5′→3′ direction:
(1) a 5′ cap;
(2) a 5′ untranslated region (UTR);
(3) a polynucleotide sequence encoding a secretory signal peptide;
(4) a polynucleotide sequence encoding at least a portion of a transmembrane and cytoplasmic domain of a major histocompatibility complex (MHC) molecule;
(5) a 3′ UTR comprising:
(a) a 3′ untranslated region of an Amino-Terminal Enhancer of Split (AES) mRNA or a fragment thereof; and
(b) non-coding RNA of a mitochondrially encoded 12S RNA or a fragment thereof; and
(6) a poly(A) sequence.

40. The RNA molecule of claim 39, further comprising a polynucleotide sequence encoding at least 1 neoepitope; wherein the polynucleotide sequence encoding the at least 1 neoepitope is between the polynucleotide sequence encoding the secretory signal peptide and the polynucleotide sequence encoding the at least portion of the transmembrane and cytoplasmic domain of the MHC molecule in the 5′→3′ direction.

41. The RNA molecule of claim 39, further comprising, in the 5′→3′ direction: a polynucleotide sequence encoding an amino acid linker; and a polynucleotide sequence encoding a neoepitope;
wherein the polynucleotide sequences encoding the amino acid linker and the neoepitope form a first linker-neoepitope module; and
wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction.

42. The RNA molecule of claim 41, wherein the amino acid linker comprises the sequence GGSGGGGSGG (SEQ ID NO:39).

43. The RNA molecule of claim 41, wherein the polynucleotide sequence encoding the amino acid linker comprises the sequence GGCGGCUCUGGAGGAGGCGGCUCCGGAGGC (SEQ ID NO:37).

44. The RNA molecule of any one of claims 41-43, further comprising, in the 5′→3′ direction: at least a second linker-epitope module, wherein the at least second linker-epitope module comprises a polynucleotide sequence encoding an amino acid linker and a polynucleotide sequence encoding a neoepitope;
wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and
wherein the neoepitope of the first linker-epitope module is different from the neoepitope of the second linker-epitope module.

45. The RNA molecule of claim 44, wherein the RNA molecule comprises 5 linker-epitope modules, and wherein the 5 linker-epitope modules each encode a different neoepitope.

46. The RNA molecule of claim 44, wherein the RNA molecule comprises 10 linker-epitope modules, and wherein the 10 linker-epitope modules each encode a different neoepitope.

47. The RNA molecule of claim 44, wherein the RNA molecule comprises 20 linker-epitope modules, and wherein the 20 linker-epitope modules each encode a different neoepitope.

48. The RNA molecule of any one of claims 40-47, further comprising a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

49. The RNA molecule of any one of claims 39-48, wherein the 5′ cap comprises a D1 diastereoisomer of the structure:

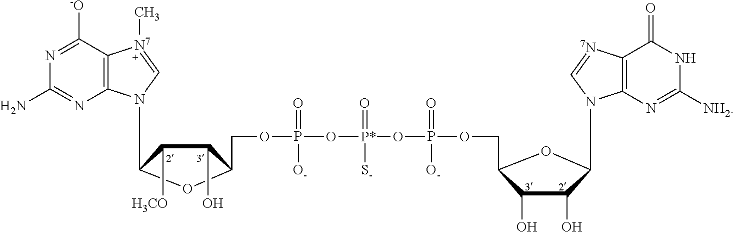

50. The RNA molecule of any one of claims 39-49, wherein the 5′ UTR comprises the sequence
(SEQ ID NO: 23)
UUCUUCUGGUCCCCACAGACUCAGAGAGAACCCGCCACC.

51. The RNA molecule of any one of claims 39-49, wherein the 5′ UTR comprises the sequence
(SEQ ID NO: 21)
GGCGAACUAGUAUUCUUCUGGUCCCCACAGACUCAGAGAGAACCCGCCA
CC.

52. The RNA molecule of any one of claims 39-51, wherein the secretory signal peptide comprises the amino acid sequence MRVMAPRTLILLLSGALALTETWAGS (SEQ ID NO:27).

53. The RNA molecule of any one of claims 39-51, wherein the polynucleotide sequence encoding the secretory signal peptide comprises the sequence
(SEQ ID NO: 25)
AUGAGAGUGAUGGCCCCCAGAACCCUGAUCCUGCUGCUGUCUGGCGCCC
UGGCCCUGACAGAGACAUGGGCCGGAAGC.

54. The RNA molecule of any one of claims 39-53, wherein the at least portion of the transmembrane and cytoplasmic domain of the MHC molecule comprises the amino acid sequence
(SEQ ID NO: 30)
IVGIVAGLAVLAVVVIGAVVATVMCRRKSSGGKGGSYSQAASSDSAQGS
DVSLTA.

55. The RNA molecule of any one of claims 39-53, wherein the polynucleotide sequence encoding the at least portion of the transmembrane and cytoplasmic domain of the MHC molecule comprises the sequence
(SEQ ID NO: 28)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCC.

56. The RNA molecule of any one of claims 39-55, wherein the 3′ untranslated region of the AES mRNA comprises the sequence
(SEQ ID NO: 33)
CUGGUACUGCAUGCACGCAAUGCUAGCUGCCCCUUUCCCGUCCUGGGUAC
CCCGAGUCUCCCCCGACCUCGGGUCCCAGGUAUGCUCCCACCUCCACCUG
CCCCACUCACCACCUCUGCUAGUUCCAGACACCUCC.

57. The RNA molecule of any one of claims 39-56, wherein the non-coding RNA of the mitochondrially encoded 12S RNA comprises the sequence
(SEQ ID NO: 35)
CAAGCACGCAGCAAUGCAGCUCAAAACGCUUAGCCUAGCCACACCCCCAC
GGGAAACAGCAGUGAUUAACCUUUAGCAAUAAACGAAAGUUUAACUAAGC
UAUACUAACCCCAGGGUUGGUCAAUUUCGUGCCAGCCACACCG.

58. The RNA molecule of any one of claims 39-57, wherein the 3′ UTR comprises the sequence
(SEQ ID NO: 31)
CUCGAGCUGGUACUGCAUGCACGCAAUGCUAGCUGCCCCUUUCCCGUCCU
GGGUACCCCGAGUCUCCCCCGACCUCGGGUCCCAGGUAUGCUCCCACCUC
CACCUGCCCCACUCACCACCUCUGCUAGUUCCAGACACCUCCCAAGCACG
CAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAA
CCCCAGGGUUGGUCAAUUUCGUGCCAGCCACACCGAGACCUGGUCCAGAG
UCGCUAGCCGCGUCGCU.

59. The RNA molecule of any one of claims 39-58, wherein the poly(A) sequence comprises 120 adenine nucleotides.

60. An RNA molecule comprising, in the 5′→3′ direction: the polynucleotide sequence
(SEQ ID NO: 19)
GGCGAACUAGUAUUCUUCUGGUCCCCACAGACUCAGAGAGAACCCGCCAC
CAUGAGAGUGAUGGCCCCCAGAACCCUGAUCCUGCUGCUGUCUGGCGCCC
UGGCCCUGACAGAGACAUGGGCCGGAAGC;
and the polynucleotide sequence
(SEQ ID NO: 20)
AUCGUGGGAAUUGUGGCAGGACUGGCAGUGCUGGCCGUGGUGGUGAUCGG
AGCCGUGGUGGCUACCGUGAUGUGCAGACGGAAGUCCAGCGGAGGCAAGG
GCGGCAGCUACAGCCAGGCCGCCAGCUCUGAUAGCGCCCAGGGCAGCGAC
GUGUCACUGACAGCCUAGUAACUCGAGCUGGUACUGCAUGCACGCAAUGC
UAGCUGCCCCUUUCCCGUCCUGGGUACCCCGAGUCUCCCCCGACCUCGGG
UCCCAGGUAUGCUCCCACCUCCACCUGCCCCACUCACCACCUCUGCUAGU
UCCAGACACCUCCCAAGCACGCAGCAAUGCAGCUCAAAACGCUUAGCCUA
GCCACACCCCCACGGGAAACAGCAGUGAUUAACCUUUAGCAAUAAACGAA
AGUUUAACUAAGCUAUACUAACCCCAGGGUUGGUCAAUUUCGUGCCAGCC
ACACCGAGACCUGGUCCAGAGUCGCUAGCCGCGUCGCU.

61. The RNA molecule of claim 60, further comprising, between the sequences of SEQ ID NO:19 and SEQ ID NO:20, a polynucleotide sequence encoding at least one neoepitope.

62. The RNA molecule of claim 60, further comprising, in the 5′→3′ direction between the sequences of SEQ ID NO:19 and SEQ ID NO:20:
(a) at least a first linker-neoepitope module, wherein the at least first linker-neoepitope module comprises a polynucleotide sequence encoding an amino acid linker and a polynucleotide sequence encoding a neoepitope; and
(b) a second polynucleotide sequence encoding an amino acid linker.

63. The RNA molecule of claim 62, comprising 5 linker-epitope modules, wherein the 5 linker-epitope modules each encode a different neoepitope.

64. The RNA molecule of claim 62, comprising 10 linker-epitope modules, wherein the 10 linker-epitope modules each encode a different neoepitope.

65. The RNA molecule of claim 62, comprising 20 linker-epitope modules, wherein the 20 linker-epitope modules each encode a different neoepitope.

66. The RNA molecule of any one of claims 60-65, further comprising a 5′ cap, wherein the 5′ cap is located 5′ to the sequence
(SEQ ID NO: 19)
GGCGAACUAGUAUUCUUCUGGUCCCCACAGACUCAGAGAGAACCCGCCAC
CAUGAGAGUGAUGGCCCCCAGAACCCUGAUCCUGCUGCUGUCUGGCGCCC
UGGCCCUGACAGAGACAUGGGCCGGAAGC.

67. The RNA molecule of claim 66, wherein the 5′ cap comprises a D1 diastereoisomer of the structure:

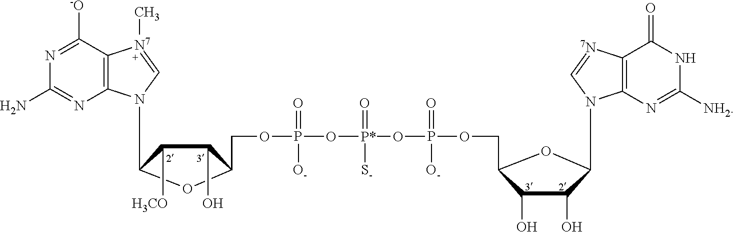

68. A liposome comprising the RNA molecule of any one of claims 39-67 and one or more lipids, wherein the one or more lipids form a multilamellar structure that encapsulates the RNA molecule.

69. The liposome of claim 68, wherein the one or more lipids comprises at least one cationic lipid and at least one helper lipid.

70. The liposome of claim 68, wherein the one or more lipids comprises (R)—N,N,N-trimethyl-2,3-dioleyloxy-1-propanaminium chloride (DOTMA) and 1,2-dioleoyl-sn-glycero-3-phosphoethanolamine (DOPE).

71. The liposome of claim 70, wherein at physiological pH the overall charge ratio of positive charges to negative charges of the liposome is 1.3:2 (0.65).

72. A method of treating or delaying progression of cancer in an individual, comprising administering to the individual an effective amount of the RNA molecule of any one of claims 39-67 or the liposome of any one of claims 68-71.

73. The method of claim 72, wherein the RNA molecule comprises one or more polynucleotides encoding one or more neoepitopes resulting from cancer-specific somatic mutations present in a tumor specimen obtained from the individual.

74. The method of claim 72 or claim 73, further comprising administering a PD-1 axis binding antagonist to the individual.

75. The method of any one of claims 72-74, wherein the cancer is selected from the group consisting of melanoma, non-small cell lung cancer, bladder cancer, colorectal cancer, triple negative breast cancer, renal cancer, and head and neck cancer.

76. The RNA molecule of any one of claims 39-67 or the liposome of any one of claims 68-71 for use in a method of treating or delaying progression of cancer in an individual.

77. A DNA molecule comprising, in the 5′→3′ direction:
(1) a polynucleotide sequence encoding a 5′ untranslated region (UTR);
(2) a polynucleotide sequence encoding a secretory signal peptide;
(3) a polynucleotide sequence encoding at least a portion of a transmembrane and cytoplasmic domain of a major histocompatibility complex (MHC) molecule;
(4) a polynucleotide sequence encoding a 3′ UTR comprising:
(a) a 3′ untranslated region of an Amino-Terminal Enhancer of Split (AES) mRNA or a fragment thereof; and
(b) non-coding RNA of a mitochondrially encoded 12S RNA or a fragment thereof; and
(5) a polynucleotide sequence encoding a poly(A) sequence.

78. The DNA molecule of claim 77, further comprising a polynucleotide sequence encoding at least one neoepitope, wherein the a polynucleotide sequence encoding the at least one neoepitope is between the polynucleotide sequence encoding the secretory signal peptide and the polynucleotide sequence encoding the at least portion of the transmembrane and cytoplasmic domain of the MHC molecule in the 5′→3′ direction.

79. The DNA molecule of claim 77, further comprising, in the 5′→3′ direction: a polynucleotide sequence encoding an amino acid linker; and a polynucleotide sequence encoding a neoepitope;
wherein the polynucleotide sequences encoding the amino acid linker and the neoepitope form a first linker-neoepitope module; and
wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction.

80. The DNA molecule of claim 79, wherein the amino acid linker comprises the sequence GGSGGGGSGG (SEQ ID NO:39).

81. The DNA molecule of claim 79, wherein the polynucleotide sequence encoding the amino acid linker comprises the sequence GGCGGCTCTGGAGGAGGCGGCTCCGGAGGC (SEQ ID NO:38).

82. The DNA molecule of any one of claims 79-81, further comprising, in the 5′→3′ direction: at least a second linker-epitope module, wherein the at least second linker-epitope module comprises a polynucleotide sequence encoding an amino acid linker and a polynucleotide sequence encoding a neoepitope;
wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and
wherein the neoepitope of the first linker-epitope module is different from the neoepitope of the second linker-epitope module.

83. The DNA molecule of claim 82, wherein the DNA molecule comprises 5 linker-epitope modules, and wherein the 5 linker-epitope modules each encode a different neoepitope.

84. The DNA molecule of claim 82, wherein the DNA molecule comprises 10 linker-epitope modules, and wherein the 10 linker-epitope modules each encode a different neoepitope.

85. The DNA molecule of claim 82, wherein the DNA molecule comprises 20 linker-epitope modules, and wherein the 20 linker-epitope modules each encode a different neoepitope.

86. The DNA molecule of any one of claims 78-85, further comprising a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

87. The DNA molecule of any one of claims 77-84, wherein the polynucleotide encoding the 5′ UTR comprises the sequence TTCTTCTGGTCCCCACAGACTCAGAGAGAACCCGCCACC (SEQ ID NO:24).

88. The DNA molecule of any one of claims 77-84, wherein the polynucleotide encoding the 5′ UTR comprises the sequence
(SEQ ID NO: 22)
GGCGAACTAGTATTCTTCTGGTCCCCACAGACTCAGAGAGAACCCGCCA
CC.

89. The DNA molecule of any one of claims 77-88, wherein the secretory signal peptide comprises the amino acid sequence MRVMAPRTLILLLSGALALTETWAGS (SEQ ID NO:27).

90. The DNA molecule of any one of claims 77-88, wherein the polynucleotide sequence encoding the secretory signal peptide comprises the sequence
(SEQ ID NO: 26)
ATGAGAGTGATGGCCCCCAGAACCCTGATCCTGCTGCTGTCTGGCGCCCT
GGCCCTGACAGAGACATGGGCCGGAAGC.

91. The DNA molecule of any one of claims 77-90, wherein the at least portion of the transmembrane and cytoplasmic domain of the MHC molecule comprises the amino acid sequence
(SEQ ID NO: 30)
IVGIVAGLAVLAVVVIGAVVATVMCRRKSSGGKGGSYSQAASSDSAQGSD
VSLTA.

92. The DNA molecule of any one of claims 77-90, wherein the polynucleotide sequence encoding the at least portion of the transmembrane and cytoplasmic domain of the MHC molecule comprises the sequence
(SEQ ID NO: 29)
ATCGTGGGAATTGTGGCAGGACTGGCAGTGCTGGCCGTGGTGGTGATCGG
AGCCGTGGTGGCTACCGTGATGTGCAGACGGAAGTCCAGCGGAGGCAAGG
GCGGCAGCTACAGCCAGGCCGCCAGCTCTGATAGCGCCCAGGGCAGCGAC
GTGTCACTGACAGCC.

93. The DNA molecule of any one of claims 77-92, wherein the polynucleotide sequence encoding the 3′ untranslated region of the AES mRNA comprises the sequence
(SEQ ID NO: 34)
CTGGTACTGCATGCACGCAATGCTAGCTGCCCCTTTCCCGTCCTGGGTAC
CCCGAGTCTCCCCCGACCTCGGGTCCCAGGTATGCTCCCACCTCCACCTG
CCCCACTCACCACCTCTGCTAGTTCCAGACACCTCC.

94. The DNA molecule of any one of claims 77-93, wherein the polynucleotide encoding the non-coding RNA of the mitochondrially encoded 12S RNA comprises the sequence
(SEQ ID NO: 36)
CAAGCACGCAGCAATGCAGCTCAAAACGCTTAGCCTAGCCACACCCCCAC
GGGAAACAGCAGTGATTAACCTTTAGCAATAAACGAAAGTTTAACTAAGC
TATACTAACCCCAGGGTTGGTCAATTTCGTGCCAGCCACACCG.

95. The DNA molecule of any one of claims 77-94, wherein the polynucleotide encoding the 3′ UTR comprises the sequence
(SEQ ID NO: 32)
CTGGTACTGCATGCACGCAATGCTAGCTGCCCCTTTCCCGTCCTGGGTAC
CCCGAGTCTCCCCCGACCTCGGGTCCCAGGTATGCTCCCACCTCCACCTG
CCCCACTCACCACCTCTGCTAGTTCCAGACACCTCCCAAGCACGCAGCAA
TGCAGCTCAAAACGCTTAGCCTAGCCACACCCCCACGGGAAACAGCAGTG
ATTAACCTTTAGCAATAAACGAAAGTTTAACTAAGCTATACTAACCCCAG
GGTTGGTCAATTTCGTGCCAGCCACACCGAGACCTGGTCCAGAGTCGCTA
GCCGCGTCGCT.

96. The DNA molecule of any one of claims 77-95, wherein the poly(A) sequence comprises 120 adenine nucleotides.

97. A DNA molecule comprising, in the 5′→3′ direction: the polynucleotide sequence
(SEQ ID NO: 40)
GGCGAACTAGTATTCTTCTGGTCCCCACAGACTCAGAGAGAACCCGCCAC
CATGAGAGTGATGGCCCCCAGAACCCTGATCCTGCTGCTGTCTGGCGCCC
TGGCCCTGACAGAGACATGGGCCGGAAGC;
and the polynucleotide sequence
(SEQ ID NO: 41)
ATCGTGGGAATTGTGGCAGGACTGGCAGTGCTGGCCGTGGTGGTGATCGG
AGCCGTGGTGGCTACCGTGATGTGCAGACGGAAGTCCAGCGGAGGCAAGG
GCGGCAGCTACAGCCAGGCCGCCAGCTCTGATAGCGCCCAGGGCAGCGAC
GTGTCACTGACAGCCTAGTAACTCGAGCTGGTACTGCATGCACGCAATGC
TAGCTGCCCCTTTCCCGTCCTGGGTACCCCGAGTCTCCCCCGACCTCGGG
TCCCAGGTATGCTCCCACCTCCACCTGCCCCACTCACCACCTCTGCTAGT
TCCAGACACCTCCCAAGCACGCAGCAATGCAGCTCAAAACGCTTAGCCTA
GCCACACCCCCACGGGAAACAGCAGTGATTAACCTTTAGCAATAAACGAA
AGTTTAACTAAGCTATACTAACCCCAGGGTTGGTCAATTTCGTGCCAGCC
ACACCGAGACCTGGTCCAGAGTCGCTAGCCGCGTCGCT.

98. The DNA molecule of claim 97, further comprising, in the 5′→3′ direction between the sequences of SEQ ID NO:40 and SEQ ID NO:41: a polynucleotide sequence encoding at least one neoepitope.

99. The DNA molecule of claim 97, further comprising, in the 5′→3′ direction between the sequences of SEQ ID NO:40 and SEQ ID NO:41:
(a) at least a first linker-neoepitope module, wherein the at least first linker-neoepitope module comprises a polynucleotide sequence encoding an amino acid linker and a polynucleotide sequence encoding a neoepitope; and
(b) a second polynucleotide sequence encoding an amino acid linker.

100. The DNA molecule of claim 99, comprising 5 linker-epitope modules, wherein the 5 linker-epitope modules each encode a different neoepitope.

101. The DNA molecule of claim 99, comprising 10 linker-epitope modules, wherein the 10 linker-epitope modules each encode a different neoepitope.

102. The DNA molecule of claim 99, comprising 20 linker-epitope modules, wherein the 20 linker-epitope modules each encode a different neoepitope.

103. A method of producing an RNA molecule, the method comprising transcribing the DNA molecule of any one of claims 77-102.

104. A method of treating or delaying progression of cancer in an individual, comprising administering to the individual the RNA molecule of any one of claims 39-67 or the liposome of any one of claims 68-71 according to the method of any one of claims 1-35.

105. A method of treating or delaying progression of cancer in an individual, comprising administering to the individual the RNA molecule of any one of claims 39-67 or the liposome of any one of claims 68-71 in combination with a PD-1 axis binding antagonist.

106. The method of claim 105, wherein the RNA molecule or liposome is administered to the individual at a dose of about 15 μg, about 25 μg, about 38 μg, about 50 μg, or about 100 μg, and wherein the PD-1 axis binding antagonist is administered to the individual at a dose of about 200 mg or about 1200 mg.

107. The method of claim 105 or claim 106, wherein the RNA molecule or liposome and the PD-1 axis binding antagonist are administered to the individual in 8 21-day Cycles.

108. The method of claim 107, wherein the PD-1 axis binding antagonist is pembrolizumab and is administered to the individual at a dose of about 200 mg on Day 1 of Cycles 1-8, and wherein the RNA molecule or liposome is administered to the individual at a dose of about 25 μg on Days 1, 8, and 15 of Cycle 2 and Day 1 of Cycles 3-7.
